(19) United States
(12) Patent Application Publication
(10) Pub. No.: US 2019/0183465 A1
(43) Pub. Date: Jun. 20, 2019

(54) VARIABLE TISSUE COLLECTION AND PROCESSING CASSETTE

(71) Applicant: Devicor Medical Products, Inc., Cincinnati, OH (US)

(72) Inventor: Bryan R. Keller, Loveland, OH (US)

(21) Appl. No.: 16/223,397

(22) Filed: Dec. 18, 2018

Related U.S. Application Data
(60) Provisional application No. 62/607,548, filed on Dec. 19, 2017.
(60) Provisional application No. 62/607,698, filed on Dec. 19, 2017.

(51) Int. Cl. A61B 10/02; A61B 10/00

(52) U.S. Cl. CPC A61B 10/0266; A61B 10/0096; A61B 10/0283

(57) ABSTRACT

An apparatus includes a tissue processing cassette and a biopsy device. The tissue processing cassette is sized to be used in a pathology laboratory for purposes of dehydrating, embedding and sectioning. The tissue processing cassette has at least one distal opening through which tissue samples are received and having a floor with a plurality of openings. The biopsy device is for cutting tissue samples and has a cassette holder configured to translate relative to a proximal end of the biopsy device. The cassette holder is adapted to receive the tissue processing cassette. The cassette holder has a fluid path adapted to be coupled to a vacuum source and be in fluid communication with the interior of the tissue processing cassette through the openings of the floor such that the vacuum from the vacuum source facilitates the transfer of the cut tissue samples into the received tissue processing cassette.

### PRIORITY

This application claims priority to U.S. Provisional Patent App. No. 62/607,548 entitled “Variable Tissue Collection and Processing Cassette,” filed on Dec. 19, 2017; and U.S. Provisional Patent App. No. 62/607,698 entitled “Remote Tissue Collection Indexing and Processing Cassette,” filed on Dec. 19, 2017, the disclosures of which are incorporated by reference herein.

### BACKGROUND

A biopsy is the removal of a tissue sample to examine tissue for signs of cancer or other disorders. Tissue samples are obtained in a variety of ways using various medical procedures involving a variety of the sample collection devices. For example, biopsies may be open (surgically removing tissue) or percutaneous (e.g. by fine needle aspiration, core needle biopsy or vacuum assisted biopsy). After the tissue sample is collected, the tissue sample is analyzed at a lab (e.g. a pathology lab, biomedical lab, etc.) that is set up to perform the appropriate tests (such as histological analysis).

Biopsy samples have been obtained in a variety of ways in various medical procedures including open and percutaneous methods using a variety of devices. For instance, some biopsy devices may be fully operable by a user using a single hand, and with a single insertion, to capture one or more biopsy samples from a patient. In addition, some biopsy devices may be tethered to a vacuum module and/or control module, such as for communication of fluids (e.g., pressurized air, saline, atmospheric air, vacuum, etc.), for communication of power, and/or for communication of commands and the like. Other biopsy devices may be fully or at least partially operable without being tethered or otherwise connected with another device. Biopsy devices may be used under stereotactic guidance, ultrasound guidance, MRI guidance, Positron Emission Mammography (“PEM” guidance), Breast-Specific Gamma Imaging (“BSGI”) guidance or otherwise.

At several steps during tissue processing using conventional techniques and instruments, it may be necessary to manually manipulate the tissue. This manual manipulation takes time and introduces the possibility of human error causing mistakes during the processing of tissue. Any human error during the processing of tissue can make the pathological examination of the tissue much more problematic to achieve the desired goal of having an accurate diagnosis. Thus, it is understood that a desired goal of modern tissue processing is the reduction of the requirement that tissue be manually manipulated.

Various devices and techniques for tissue handling are disclosed in International Pat. Pub. No. WO 2013/192606, entitled “Biopsy Tissue Sample Transport Device and Method of Using Thereof,” published on Dec. 27, 2013; International Pat. Pub. No. WO 2013/192607, entitled “Tissue Sample Container and Methods,” published on Dec. 27, 2013; International Pat. Pub. No. WO 2014/151603, entitled “Biopsy Device,” published on Sep. 25, 2014; U.S. Pat. No. 7,715,523, entitled “System and Apparatus for Rapid Stereotactic Breast Biopsy Analysis,” issued on May 11, 2010; U.S. Pat. No. 8,503,602, entitled “System and Apparatus for Rapid Stereotactic Breast Biopsy Analysis,” issued on Aug. 6, 2013; U.S. Pat. No. 8,485,987, entitled “Tissue Handling System with Reduced Operator Exposure,” issued Jul. 16, 2016; U.S. Pat. No. 8,802,034, “Tissue Container for Molecular and Histology Diagnostics Incorporating a Breakable Membrane,” issued on Aug. 12, 2014; and U.S. Pat. No. 9,056,317, “Tissue Container for Molecular and Histology Diagnostics Incorporating a Breakable Membrane,” issued on Jun. 16, 2016. The disclosure of each of the above-cited U.S. Patents is incorporated by reference herein.

While several systems and methods have been made and used for obtaining and processing a biopsy sample, it is believed that no one prior to the inventor has made or used the invention described in the appended claims.

### BRIEF DESCRIPTION OF THE DRAWINGS

While the specification concludes with claims which particularly point out and distinctly claim the invention, it is believed the present invention will be better understood from the following description of certain examples taken in conjunction with the accompanying drawings, in which like reference numerals identify the same elements. In the drawings some components or portions of components are shown in phantom as depicted by broken lines.

FIG. 1 depicts a perspective view of an exemplary biopsy device;

FIG. 2 depicts an exploded perspective view of a tissue sample holder assembly of the biopsy device of FIG. 1;

FIG. 3 depicts a perspective view of a tissue sample tray of the tissue sample holder assembly of FIG. 2, with the tissue sample tray in an arcuate configuration;

FIG. 4 depicts a perspective view of the tissue sample tray of FIG. 3 in a flattened configuration;

FIG. 5 depicts a front elevational view of the tissue sample tray of FIG. 3 disposed within ajar;

FIG. 6 depicts a perspective view of an exemplary sample cassette for use in processing tissue sample collected with the biopsy device of FIG. 1;

FIG. 7 depicts a flowchart of an exemplary tissue collection and analysis work flow for use with the biopsy device of FIG. 1 and the sample cassette of FIG. 6;

FIG. 8 depicts a perspective view of an exemplary cassette assembly that may be readily used with the biopsy device of FIG. 1 in lieu of the tissue sample tray of FIG. 3 and/or the sample cassette of FIG. 6;

FIG. 9 depict another perspective view of the cassette assembly of FIG. 8;

FIG. 10 depicts an exploded perspective view of the cassette assembly of FIG. 8;

FIG. 11 depicts a perspective view of an exemplary cassette tray of the cassette assembly of FIG. 8;

FIG. 12 depicts another perspective view of the cassette tray of FIG. 11;

FIG. 13 depicts a perspective view of an exemplary cover of the cassette assembly of FIG. 8;

FIG. 14 depicts another perspective view of the cover of FIG. 13;

FIG. 15A depicts a side cross-sectional view of the cassette assembly of FIG. 8, with the cassette tray of FIG. 11 initially inserted into the cover of FIG. 13;

FIG. 15B depicts another side cross-sectional view of the cassette assembly of FIG. 8, with the cassette tray of FIG. 11 intermediately inserted into the cover of FIG. 13;

FIG. 15C depicts still another side cross-sectional view of the cassette assembly of FIG. 8, with the cassette tray of FIG. 11 fully inserted into the cover of FIG. 13;

FIG. 16 depicts a perspective view of an exemplary alternative tissue sample holder assembly that can be readily incorporated into the biopsy device of FIG. 1; with an exemplary manifold including the cassette assembly of FIG. 8 therein;

FIG. 17 depicts another perspective view of the tissue sample holder assembly of FIG. 16;

FIG. 18 depicts a perspective view of an exemplary actuator of the tissue sample holder assembly of FIG. 16;

FIG. 19 depicts a perspective view of the tissue sample holder assembly of FIG. 16 with the manifold removed;

FIG. 20 depicts a perspective view of the manifold of FIG. 16 receiving the cassette assembly of FIG. 8;

FIG. 21 depicts a perspective view of the manifold of FIG. 16 including a series of access openings;

FIG. 22 depicts a cross-sectional view of the tissue sample holder assembly of FIG. 16 with the manifold engaged therein, the cross section taken along lines 22-22 of FIG. 16;

FIG. 23 depicts a perspective view of the cassette assembly of FIG. 8 received in the manifold of FIG. 16, with the manifold of FIG. 16 directed into the tissue sample holder assembly of FIG. 16;

FIG. 24A depicts a front elevational view of the manifold of FIG. 16 directed toward the tissue sample holder assembly of FIG. 16, with the tissue sample holder assembly in a load state;

FIG. 24B depicts another front elevational view of the manifold of FIG. 16 received by the tissue sample holder assembly of FIG. 16, with the tissue sample holder assembly in a load state and the manifold in a load position;

FIG. 24C depicts still another front elevational view of the manifold of FIG. 16 received by the tissue sample holder assembly of FIG. 16, with the tissue sample holder assembly in a first state and the manifold transitioned to a first position;

FIG. 24D depicts yet another front elevational view of the manifold of FIG. 16 received by the tissue sample holder assembly of FIG. 16, with the tissue sample holder assembly in a second state and the manifold transitioned to a second position;

FIG. 24E depicts yet another front elevational view of the manifold of FIG. 16 received by the tissue sample holder assembly of FIG. 16, with the tissue sample holder assembly in a third state and the manifold transitioned to a third position;

FIG. 24F depicts yet another front elevational view of the manifold of FIG. 16 received by the tissue sample holder assembly of FIG. 16, with the tissue sample holder assembly in a fourth state and the manifold transitioned to a fourth position;

FIG. 24G depicts yet another front elevational view of the manifold of FIG. 16 received by the tissue sample holder assembly of FIG. 16, with the tissue sample holder assembly in an eject state and the manifold transitioned to an eject position;

FIG. 24H depicts a front elevational view of the manifold of FIG. 16 removed from the tissue sample holder assembly of FIG. 16, with the tissue sample holder assembly in an eject state and the manifold removably ejected;

FIG. 25A depicts a cross-sectional view of the manifold of FIG. 16 received by the tissue sample holder assembly of FIG. 16, with the tissue sample holder assembly in the first state and the manifold in the first position, the cross section taken along line 22-22 of FIG. 16;

FIG. 25B depicts another front elevational view of the manifold of FIG. 16 received by the tissue sample holder assembly of FIG. 16, with the tissue sample holder assembly in the second state and the manifold in the second position, the cross section taken along line 22-22 of FIG. 16;

FIG. 25C depicts still another front elevational view of the manifold of FIG. 16 received by the tissue sample holder assembly of FIG. 16, with the tissue sample holder assembly in the third state and the manifold in the third position, the cross section taken along line 22-22 of FIG. 16;

FIG. 25D depicts yet another front elevational view of the manifold of FIG. 16 received by the tissue sample holder assembly of FIG. 16, with the tissue sample holder assembly in the fourth state and the manifold in the fourth position, the cross section taken along line 22-22 of FIG. 16;

FIG. 26 depicts a perspective view of an exemplary manifold cover including a screened surface;

FIG. 27A depicts a perspective view of the manifold cover of FIG. 26 directed toward the cassette assembly of FIG. 8;

FIG. 27B depicts another perspective view of the manifold cover of FIG. 26 engaged to the cassette assembly of FIG. 8;

FIG. 28 depicts still another perspective view of the manifold cover of FIG. 26 engaged to the cassette assembly of FIG. 8;

FIG. 29 depicts a perspective view of an exemplary alternative manifold cover including a sealed surface;

FIG. 30 depicts a perspective view of the manifold cover of FIG. 28 engaged to the cassette assembly of FIG. 8;

FIG. 31 depicts a side elevational view of an exemplary remote tissue collection assembly connecting the biopsy device of FIG. 1 with the tissue sample holder assembly of FIG. 16;

FIG. 32 depicts a partial side elevational view of the remote tissue collection assembly of FIG. 30 connecting the biopsy device of FIG. 1 to the tissue sample holder assembly of FIG. 16;

FIG. 33 depicts a perspective view of an exemplary probe adapter that can be readily incorporated with the biopsy device of FIG. 1;

FIG. 34 depicts a perspective view of an exemplary tissue sample holder adapter that can be readily incorporated with the tissue sample holder assembly of FIG. 16;

FIG. 35 depicts a side elevational view of the remote tissue collection assembly of FIG. 30 connecting the biopsy device of FIG. 1 and the tissue sample holder assembly of FIG. 2;

FIG. 36 depicts a partial side elevational view of the remote tissue collection assembly of FIG. 33 connecting the biopsy device of FIG. 1 to the tissue sample holder assembly of FIG. 2;

FIG. 37 depicts a perspective view of an exemplary alternative tissue sample holder adapter that can be readily incorporated with the tissue sample holder assembly of FIG. 2;

FIG. 38 depicts a cross sectional view of the remote tissue collection assembly of FIG. 33 connecting the biopsy device of FIG. 1 to the tissue sample holder assembly of FIG. 2;

FIG. 39 depicts a perspective view of an exemplary tissue sample holder assembly engaged with a coupler of the biopsy device of FIG. 1;

FIG. 40 depicts a perspective view of the tissue sample holder assembly of FIG. 39 disengaged from the coupler of the biopsy device of FIG. 1;

FIG. 41 depicts an exploded perspective view of the tissue sample holder assembly of FIG. 39, the tissue sample holder assembly including a manifold with a manifold insert and a seal contained therein;

FIG. 42 depicts a perspective view of the tissue sample holder assembly of FIG. 39, with the manifold insert including multiple openings;

FIG. 43 depicts a perspective view of an exemplary cassette insert;

FIG. 44 depicts a top view of the cassette insert of FIG. 43;

FIG. 45A depicts a perspective view of the sample cassette of FIG. 6 receiving the cassette insert of FIG. 43;

FIG. 45B depicts a perspective view of the tissue sample holder assembly of FIG. 39 receiving the sample cassette of FIG. 6 and the cassette insert of FIG. 43;

FIG. 46 depicts a partial perspective view of the tissue sample holder assembly of FIG. 8 with the sample cassette of FIG. 6 and cassette insert of FIG. 43 positioned within the manifold insert;

FIG. 47A depicts a perspective view of the assembly of FIG. 45B laterally advanced towards the coupler of the biopsy device of FIG. 1;

FIG. 47B depicts a perspective view of the assembly of FIG. 45B slidably engaged with the coupler of the biopsy device of FIG. 1, the assembly being in a first position;

FIG. 47C depicts a perspective view of the assembly of FIG. 45B slidably engaged with the coupler of the biopsy device of FIG. 1, the assembly being in a second position;

FIG. 47D depicts a perspective view of the assembly of FIG. 45B slidably engaged with the coupler of the biopsy device of FIG. 1, the assembly being in a third position;

FIG. 47E depicts a perspective view of the assembly of FIG. 45B disengaged from the coupler of the biopsy device of FIG. 1;

FIG. 48A depicts a top plan view of the assembly of FIG. 45B in the first position;

FIG. 48B depicts a top plan view of the assembly of FIG. 45B in the second position;

FIG. 48C depicts a top plan view of the assembly of FIG. 45B in the third position;

FIG. 49 depicts a cross sectional view of the assembly of FIG. 47B, with tissue samples extracted from the biopsy device of FIG. 1 deposited within the tissue sample holder assembly of FIG. 39, the cross section taken along line 49-49 of FIG. 47B;

FIG. 50 depicts a perspective view of another exemplary tissue sample holder assembly slidably engaged to a coupler of the biopsy device of FIG. 1;

FIG. 51 depicts a perspective view of the tissue sample holder assembly of FIG. 50 disengaged from the coupler of the biopsy device of FIG. 1;

FIG. 52 depicts a perspective view of the tissue sample holder assembly of FIG. 50 including a manifold that has an opening and a vent opening;

FIG. 53 depicts a partial perspective view of the tissue sample holder assembly of FIG. 50, with the opening of the manifold extending into an inner chamber;

FIG. 54 depicts a perspective view of the tissue sample holder assembly of FIG. 50 including a removable drawer, the removable drawer receiving the sample cassette of FIG. 6 therein;

FIG. 55A depicts a cross sectional view of the tissue sample holder assembly of FIG. 50 slidably receiving the drawer and sample cassette of FIG. 6 within the manifold, the cross section taken along line 55-55 of FIG. 50;

FIG. 55B depicts a cross sectional view of the tissue sample holder assembly of FIG. 50 slidably receiving the drawer and sample cassette of FIG. 6 within the manifold, with the sample cassette engaging a ramp of the manifold, the cross section taken along line 55-55 of FIG. 50;

FIG. 55C depicts a cross sectional view of the tissue sample holder assembly of FIG. 50 with the drawer and sample cassette of FIG. 6 received within the manifold, the sample cassette securely engaged to the ramp of the manifold, the cross section taken along line 55-55 of FIG. 50;

FIG. 56 depicts a partial perspective view of the tissue sample holder assembly of FIG. 50 with the drawer and sample cassette of FIG. 6 received within the manifold, the sample cassette securely engaged to the ramp of the manifold;

FIG. 57 depicts a cross sectional view of the tissue sample holder assembly of FIG. 50, with tissue samples extracted from the biopsy device of FIG. 1 deposited within the sample cassette of FIG. 6, the cross section taken along line 57-57 of FIG. 56;

FIG. 58 depicts a perspective view of another exemplary tissue sample holder assembly integrally formed with a coupler of the biopsy device of FIG. 1;

FIG. 59 depicts an exploded perspective view of the tissue sample holder assembly of FIG. 58 and the coupler of the biopsy device of FIG. 1;

FIG. 60A depicts a perspective view of the tissue sample holder assembly of FIG. 58 slidably receiving the sample cassette of FIG. 6 and cassette insert of FIG. 43 therein;

FIG. 60B depicts a perspective view of the tissue sample holder assembly of FIG. 58 with the sample cassette of FIG. 6 and cassette insert of FIG. 43 received therein, the tissue sample holder assembly being in a first position;

FIG. 60C depicts a perspective view of the tissue sample holder assembly of FIG. 58 with the sample cassette of FIG. 6 and cassette insert of FIG. 43 received therein, the tissue sample holder assembly rotated to a second position;

FIG. 60D depicts a perspective view of the tissue sample holder assembly of FIG. 58 with the sample cassette of FIG. 6 and cassette insert of FIG. 43 received therein, the tissue sample holder assembly rotated to a third position;

FIG. 61 depicts a cross sectional view of the tissue sample holder assembly of FIG. 58 with the tissue sample holder assembly in the second position, the cross section taken along line 61-61 of FIG. 60C; and

FIG. 62 depicts a cross sectional view of the assembly of FIG. 60B, with tissue samples extracted from the biopsy device of FIG. 1 deposited within the sample cassette of FIG. 6, the cross section taken along line 62-62 of FIG. 60B.

The drawings are not intended to be limiting in any way, and it is contemplated that various embodiments of the invention may be carried out in a variety of other ways, including those not necessarily depicted in the drawings. The accompanying drawings incorporated in and forming a part of the specification illustrate several aspects of the present invention, and together with the description serve to explain the principles of the invention; it being understood, however, that this invention is not limited to the precise arrangements shown.

### DETAILED DESCRIPTION

The following description of certain examples of the invention should not be used to limit the scope of the present invention. Other examples, features, aspects, embodiments, and advantages of the invention will become apparent to those skilled in the art from the following description, which is by way of illustration, one of the best modes contemplated for carrying out the invention. As will be realized, the invention is capable of other different and obvious aspects, all without departing from the invention. Accordingly, the drawings and descriptions should be regarded as illustrative in nature and not restrictive.

### I. Exemplary Biopsy Device

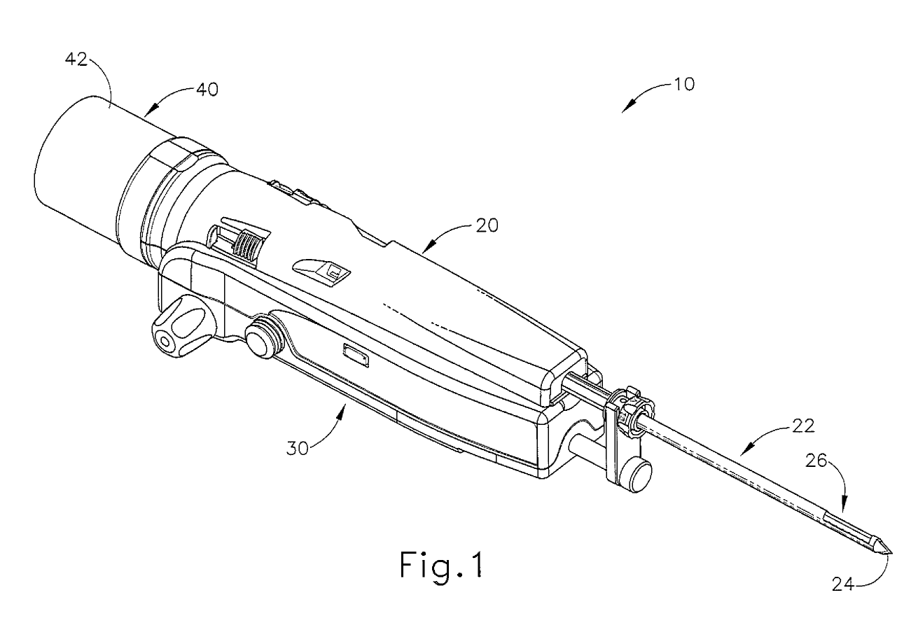

FIG. 1 depicts an exemplary biopsy device (10) that can be used to acquire tissue samples from a patient. Biopsy device (10) comprises a probe assembly (20), a holster assembly (30), and a tissue sample holder assembly (40). Probe assembly (20) includes a distally projecting needle (22) that has a tissue piercing tip (24) and a lateral aperture (26) that is located proximal to tip (24). A tubular cutter (not shown) is slidably disposed in needle (22) and is operable to sever tissue that is protruding through lateral aperture (26). The severed tissue samples are communicated proximally through the lumen of the cutter to tissue sample holder assembly (40), as described below. In some versions, probe assembly (20) is coupled with a control module that is operable to provide communication of vacuum, saline, and/or atmospheric air to probe assembly (20).

Holster assembly (30) includes features that are operable to drive the cutter, features that are operable to fire needle (22) distally into tissue, and features that are operable to rotate needle (22) about a longitudinal axis of needle (22). In some versions, holster assembly (30) is coupled with a control module via a cable that is operable to provide electrical power and/or other electrical signals to holster assembly (30). In addition, or in the alternative, holster assembly (30) may receive a pressurized medium (e.g., air, hydraulic fluid, etc.) in order to provide motive force to drive the cutter of probe assembly (20).

In the present example, probe assembly (20) and holster assembly (30) are configured for use in a stereotactic image guided biopsy procedure. By way of example only, probe assembly (20) and holster assembly (30) may be constructed and operable in accordance with at least some of the teachings of U.S. Pub. No. 2014/0039343, entitled “Biopsy System,” published Feb. 6, 2014, the disclosure of which is incorporated by reference herein. Alternatively, probe assembly (20) and holster assembly (30) may be configured for use in (or otherwise be used in) an ultrasound image guided biopsy procedure and/or an MRI guided biopsy procedure. By way of further example only, probe assembly (20) and holster assembly (30) may be constructed and operable in accordance with at least some of the teachings of U.S. Pub. No. 2013/0150751, entitled “Biopsy Device with Slide-In Probe,” published Jun. 13, 2013, the disclosure of which is incorporated by reference herein. Alternatively, probe assembly (20) and holster assembly (30) may be constructed and operable in any other suitable fashion.

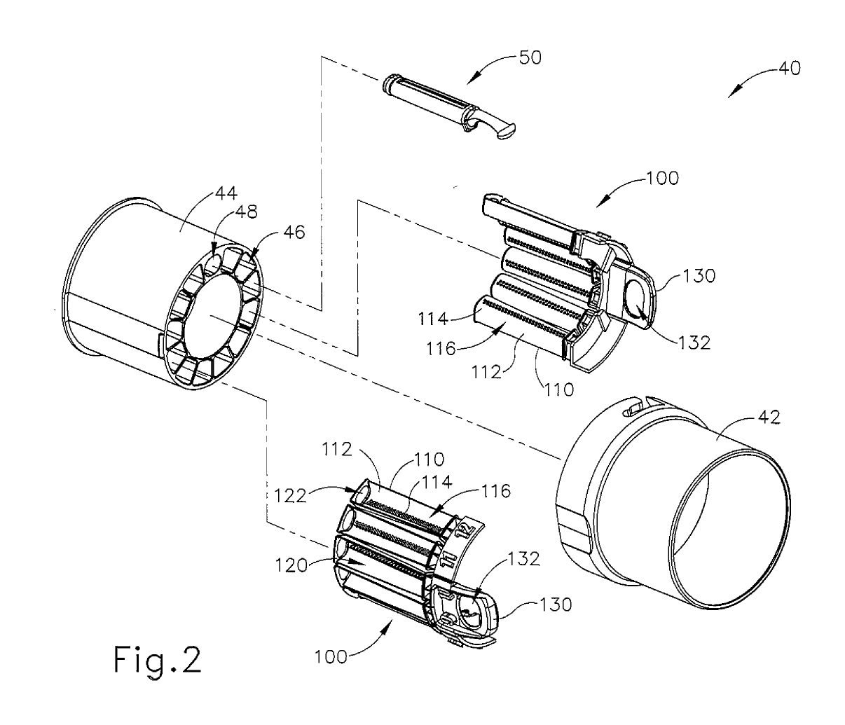

As noted above, tissue sample holder assembly (40) is configured to receive tissue samples that are severed by the cutter from tissue protruding through lateral aperture (26). As shown in FIG. 2, tissue sample holder assembly (40) of this example comprises a cylindraceous outer cover (42) that is removably coupled with probe assembly (20). A rotatable (44) member is rotatably positioned within cover (42). Rotatable member (44) defines an angularly spaced array of strip receiving chambers (46) and a plug chamber (48), such that chambers (46, 48) together an annular arrangement. Rotatable member (44) is rotatable relative to probe assembly (20) to selectively index chambers (46, 48) relative to the cutter. In some versions, drive components in holster assembly (30) drive rotation of rotatable member (44). In some other versions, rotatable member (44) is driven manually by the operator manually grasping some portion of tissue sample holder assembly (40).

As also shown in FIG. 2, tissue sample holder assembly (40) further includes a pair of tissue sample trays (100). Each tissue sample tray (100) comprises a set of distally projecting tissue sample strips (110). Each tissue sample strip (110) is configured for removable insertion into a corresponding strip receiving chamber (46) of rotatable member (44). Each tissue sample strip (110) comprises a set of strip sidewalls (112) joined by a floor (114). Strip sidewalls (112) and floor (114) cooperate to define a tissue receiving chamber (120), such that each tissue sample strip (110) is configured to receive a corresponding tissue sample. Floor (114) defines a plurality of openings (116) that are sized to provide communication of suction and fluids therethrough, while preventing communication of tissue samples therethrough. It should be understood that suction may be communicated through strip receiving chambers (46) to reach tissue receiving chambers (120) via openings (116). Each tissue sample strip (110) of the present example also includes a distal opening (122). Distal opening (122) is sized and configured to enable a severed tissue sample to pass therethrough in order for the tissue sample to be deposited into tissue receiving chamber (120).

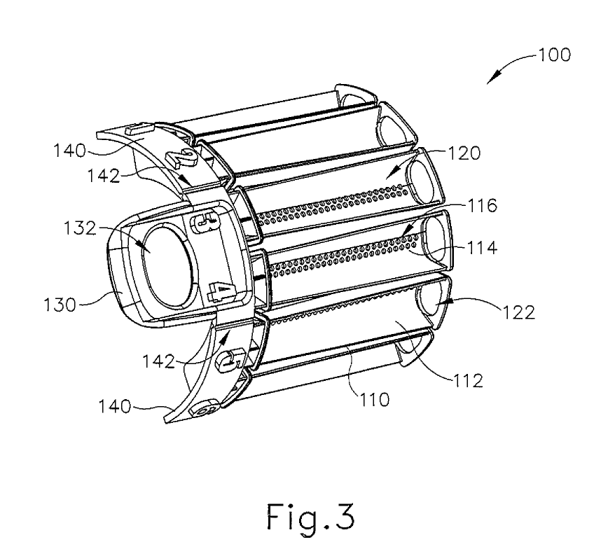

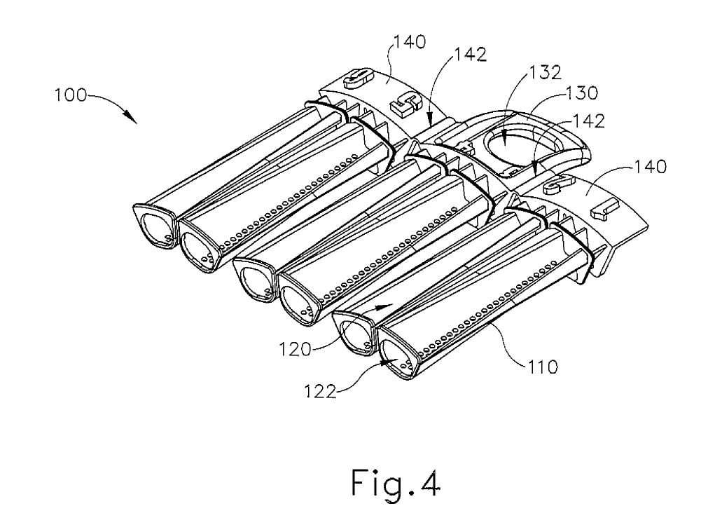

As best seen in FIGS. 3-4, each tissue sample tray (100) further includes a proximally projecting pull tab (130) that defines a tab opening (132). Pull tab (130) is configured to facilitate grasping of tissue sample tray (100) by an operator. Tissue sample tray (100) also includes a set of proximal panels (140). In the present example, two tissue sample strips (110) project distally relative to a corresponding panel (140) of the set of panels (140). Pull tab (130) projects proximally from the centrally positioned panel (140). Panels (140) are flexibly joined together by living hinges (142). Living hinges (142) enable tissue sample tray (100) to transition between the arcuate configuration shown in FIG. 3 and the flattened configuration shown in FIG. 4. In the arcuate configuration, tissue sample tray (100) is configured to fit in rotatable member (44). In the flattened configuration, tissue sample tray (100) is configured to fit in a container (200) as will be described in greater detail below.

As noted above, rotatable member (44) is rotatable relative to probe assembly (20) to selectively index strip receiving chambers (46) relative to the cutter, to thereby selectively index tissue receiving chambers (120) of tissue sample strips (110) relative to the cutter. Rotatable member (44) is also operable to index plug receiving chamber (48) relative to the cutter. When rotatable member (44) is angularly positioned to index plug receiving chamber (48) relative to the cutter, plug (50) may be removed from plug receiving chamber (48) to enable insertion of a biopsy site marker applier instrument (or some other kind of instrument) through the cutter and needle assembly (22), thereby providing an access path to the biopsy site via lateral aperture (26). Otherwise, plug (50) may be left in plug receiving chamber (48) during operation of biopsy device (10), thereby sealing plug receiving chamber (48).

By way of example only, tissue sample holder (40) may be configured and operable in accordance with at least some of the teachings of U.S. Pub. No. 2014/0039343, entitled “Biopsy System,” published Feb. 6, 2014, the disclosure of which is incorporated by reference herein and/or U.S. Pub. No. 2014/0275999, entitled “Biopsy Device,” published Sep. 18, 2014, the disclosure of which is incorporated by reference herein.

In some instances it may be desirable to insert tissue sample tray (100) in a preservative or other protective medium after collecting tissue samples within each tissue receiving chamber (120) of tissue sample tray (100). As seen in FIG. 5, in some examples tissue sample tray (100) may be used in connection with jar (160). Jar (160) is generally configured to receive one or more tissue sample trays (100) after collection of tissue samples using biopsy device (10). As will be described in greater detail below, jar (160) may be used to transport or store tissue samples once one or more tissue sample trays (100) are deposited therein.

In the present example, jar (160) includes a cup (162) and a lid (166). Cup (162) defines a reservoir (164), which can be used to contain fluid within cup (162). Cup (162) defines a generally cylindrical shape that is sized to receive one or more tissue sample trays (100). Lid (166) generally corresponds to the cylindrical shape of cup (162). Lid (166) is further configured to be selectively fastened onto a top portion of cup (162). In the present example, lid (166) includes seals or other features configured to seal cup (162) relative to the exterior of cup (162). As described above, reservoir (164) is generally configured to hold fluid. Thus, lid (166) is corresponding configured to hold the fluid within cup (162).

As described above, jar (160) is generally filled with fluid. Thus, when tissue sample tray (100) is disposed within jar (160), tissue sample tray (100) is generally at least partially submerged in fluid. In the present example, fluid is generally configured to act as a preservative of tissue samples contained within tissue sample tray (100). By way of example only, one suitable preservative may include formalin. However, it should be understood that in other examples numerous alternative fluids as will be apparent to those of ordinary skill in the art in view of the teachings herein.

### II. Exemplary Tissue Processing Cassette

Once tissue samples have been collected using biopsy device (10) or other similar devices described herein, it may be desirable to subject such tissue samples to further pathological analysis. To facilitate such analysis, such tissue samples may be subjected to a variety of processing steps described in greater detail below. During these processing steps, it may be desirable to dispose the collected tissue samples within a container or other device to help segregate and track the collected tissue samples relative to other tissue sample collected from the same or other patients as well as the same or other biopsy procedures.

FIG. 6 shows an exemplary tissue processing cassette (200) that may be used in connection with biopsy device (10) to store and track tissue samples after collection via biopsy device (10). Tissue processing cassette (200) is generally configured to receive and enclose a plurality of tissue samples therein. As can be seen, tissue processing cassette (200) comprises a base (210) and a lid (230). Base (210) comprises a distal wall (212), a proximal wall (216), a pair of sidewalls (220), and a floor (222). Base (210) further includes a labeling surface (226) extending distally from distal wall (212).

Walls (212, 216, 220) are generally connected to form a rectangular pattern around floor (422). Each wall is generally solid, thereby forming a sample chamber (228) therein. As will be described in greater detail below, sample chamber (228) is generally configured to contain tissue samples within tissue processing cassette (200) when lid (230) is closed relative to base (210).

Distal wall (212) and proximal wall (216) each include a lid receiver (214, 218). Each lid receiver (214, 218) is generally configured to receive at least a portion of lid (230) to thereby selectively secure lid (230) to base (210). Although not shown, it should be understood that each lid receiver (214, 218) can include certain fastening features to facilitate securing lid (230) to base (210). As will be described in greater detail below, these fastening features generally facilitate a snap fit coupling mechanism. However, it should be understood that in other examples alternative coupling mechanisms may be used such as compression fit mechanisms, or any other suitable coupling mechanism as will be apparent to those of ordinary skill in the art in view of the teachings herein.

As described above, labeling surface (226) protrudes distally from distal wall (212). Labeling surface (226) is generally configured to receive a label to provide information to an operator related to the samples contained within tissue processing cassette (200). Although labeling surface (226) of the present example can receive a label (e.g., a pre-printed self-adhering label), it should be understood labeling surface (226) is also configured to permit direct printing of a label onto labeling surface (226). For instance, in some examples labels are laser etched onto labeling surface (226) using a printer configured to receive tissue processing cassette (200) and thereby print directly onto labeling surface (226). To facilitate such printing, it should be understood that labeling surface (226) can also be equipped with a colored coating that can be etched away by the printer described above.

Floor (222) includes a plurality of vents (224) arranged in an array across the surface of floor (222). Vents (224) are generally configured to promote the flow of fluid through floor (222), yet maintain tissue samples within sample chamber (228). To facilitate this configuration, vents (224) have a narrow rectangular form. In other examples, vents (224) can be configured with a variety of alternative shapes such as round, oval-shaped, square, and/or etc. Although vents (224) in the present example are arranged to uniformly occupy the entire surface of floor (222), it should be understood that in other examples vents (224) can be arranged in a variety of other ways. For instance, vents (224) can be isolated to a specific region or multiple regions of floor (222). Of course, other alternative arrangements for vents (224) will be apparent to those of ordinary skill in the art in view of the teachings herein.

Lid (230) comprises a cover portion (234) that is generally configured to engage base (210) to hold tissue samples within sample chamber (228) of base (210). Lid (230) further includes a lip (238) protruding from cover portion (234). Lip (238) extends around the perimeter of cover portion (234) defining a rectangular shape that corresponds to the rectangular shape defined by walls (212, 216, 220) of base (210). As will be understood, lip (238) is generally configured to fit within sample chamber (228) adjacent to each wall (212, 216, 220) to laterally secure and locate cover portion (234) relative to base (210) when lid (230) is in a closed position relative to base (210).

Like with floor (222) described above, cover portion (234) likewise includes a plurality of vents (236) arranged in an array across the surface of cover portion (234). Like vents (224) described above, vents (236) are generally configured to promote the flow of fluid through cover portion (234), yet maintain tissue samples within sample chamber (228). To facilitate this configuration, vents (236) have a narrow rectangular form. In other examples, vents (236) can be configured with a variety of alternative shapes such as round, oval-shaped, square, and/or etc. Although vents (236) in the present example are arranged to uniformly occupy the entire surface of cover portion (234), it should be understood that in other examples vents (236) can be arranged in a variety of other ways. For instance, vents (236) can be isolated to a specific region or multiple regions of cover portion (234). Of course, other alternative arrangements for vents (234) will be apparent to those of ordinary skill in the art in view of the teachings herein.

Lid (230) further comprises a proximal fastener (240) and distal fastener (242). Proximal fastener (240) is configured to engage lid receiver (218) of proximal wall (216), while distal fastener (242) is configured to engage lid receiver (214) of distal wall (212). Each fastener (240, 242) includes a tooth, lip, or other engagement feature that mates with a corresponding feature of each lid receiver (214, 218). As described above, this generally provides a snap fit coupling between each fastener (240, 242) and each lid receiver (216, 218) to selectively maintain lid (230) in the closed position.

Lid (230) is secured to proximal wall (216) of base (210) with an integral living hinge (232). Living hinge (232) permits pivoting of lid (230) relative to base (210) such that lid (230) may move between an open position (e.g., FIG. 6) and the closed position. This configuration permits tissue samples to be loaded into sample chamber (228) when lid (230) is in the open position. Lid (230) can then be pivoted to the closed position to secure the loaded tissue samples within sample chamber (228). To assist pivoting of lid (230), lid (230) further includes a manipulator (244) or thumb snap. Manipulator (244) generally protrudes distally from cover portion (234) to provide a gripping feature when lid (230) is in the closed position. This facilitates moving lid (230) from the closed position to the open position by providing a surface for an operator to grasp.

As described above, tissue samples may be subjected to various processing and or analysis steps after the tissue samples are collected with biopsy device (10) or other suitable devices. During such steps, tissue processing cassette (200) can be used to facilitate transport, tracking, and storage of the collected tissue samples. In particular, FIG. 7 shows a generally workflow associated with biopsy device (10) and tissue processing cassette (200) described above. It should be understood that the workflow (300) shown in FIG. 7 and the description herein is only exemplary and that various alternative procedural steps may be used in addition and/or in the alternative to the steps shown in FIG. 7. For instance, in some examples biopsy device (10) and/or tissue processing cassette (200) may be used in accordance with one or more of the teachings of U.S. Ser. No. 15/638,843, entitled “Integrated Workflow for Processing Tissue Samples from Breast Biopsy Procedures,” filed on Jun. 30, 2017, the disclosure of which is incorporated by reference herein.

In the workflow (300) shown in FIG. 7, tissue samples are collected during a biopsy procedure represented by box (310). During the biopsy procedure in box (310), biopsy device (10) may be used to collect a plurality of tissue samples into one or more tissue sample trays (100). Although the description above is primarily related to collection of tissue samples using a stereotactic biopsy procedure, it should be understood that various alternative procedures can be used such as ultrasonically guided procedures, Mill guided procedures, and/or etc. In addition, although the description above is primarily related to tissue sample collection using a multi-chamber-style tissue sample holder similar to tissue sample holder assembly (40), it should be understood that various alternative tissue sample collection devices may be used such as basket-style tissue sample holders. Alternatively, tissue samples can be collected without a tissue sample holder and may be merely plucked from a sample surface on a device similar to biopsy device (10).

Regardless of the particular process for collecting tissue samples, once tissue samples are collected, they may be subjected to procedure room x-ray as shown in box (320). During procedure room x-ray, an operator uses x-ray imaging in the procedure room to perform preliminary analysis on the collected tissue samples. During this stage, the collected tissue samples are primarily analyzed using x-ray imaging to determine if any one or more of the collected tissue samples include calcifications or other suspicious features identifiable via x-ray. After this preliminary analysis, more tissue samples can be acquired, if an operator is not satisfied with the preliminary analysis. Alternatively, an operator may be satisfied with the originally collected tissue samples and move to the next step in the procedure.

After an operator is satisfied with preliminary procedure room x-ray analysis, the operator may insert tissue sample tray (100) or just the tissue samples into jar (160). As described above, jar (160) may be filled with formalin or other fluids to preserve the collected tissue samples for storage and/or transport as represented by box (330). Jar (160) is then transported to a pathology laboratory so that the tissue samples can be subjected to further analysis as represented by box (340).

Once jar (160) is received by the pathology laboratory, the samples can be subjected to accessioning as represented by box (342). Accessioning (342) used herein refers to the process of documenting the chain of custody of the collected tissue samples. It should be understood that this may include a variety of steps. For instance, in some examples, jar (160) can include a label that can be used to store, present, display, or otherwise provide patient information. This label can be printed during or after the biopsy procedure described above and represented by box (310). The label can then be adhered to jar (160) prior to transport to pathology as represented by box (310). Once jar (160) is received by pathology, an operator can record, scan, or otherwise collect information from the label to track the chain of custody of the collected tissue samples contained within jar (160).

Once accessioning is complete, the collected tissue samples are strained from the fluid contained within jar (160) as represented by box (350). The collected tissue samples then undergo gross examination by an operator as represented by box (360). Gross examination can include visual inspection of the collected tissue samples, palpitation of the collected tissue samples, and/or manipulating the collected tissue samples into a desired position. Preliminary observations can then be documented in a written record by an operator. Such written records can then be associated with the label described above with respect to accessioning and box (342).

After gross examination or during gross examination, the collected tissue samples are inserted into one or more tissue sample processing cassettes similar to tissue processing cassette (200) described above as represented by box (370). For instance, in the context of tissue processing cassette (200), each collected tissue sample is generally laid on floor (222) of base (210) longitudinally between distal wall (212) and proximal wall (216). Lid (230) is then pivoted to the closed position to enclose the collected tissue samples within sample chamber (228) of base (210). To promote tracking of the collected tissue samples, the tissue processing cassette can be labeled at this stage by either direct printing or adhering a self-adhering label to a structure similar to labeling surface (226) described above. This label can include certain patient information corresponding to the label described above with respect to accessioning and box (342).

Once the collected tissue samples are disposed within a tissue processing cassette similar to tissue processing cassette (200), the collected tissue samples are subjected to fixation as represented by box (380). The term fixation used herein refers to the process of using a fixative to preserve specimen integrity and to maintain the shape of cells. Generally, this process involves submerging the collected tissue samples within a fixative. One common fixative is 10% neutral buffered formalin, although other fixatives can be used. The collected tissue samples can be maintained within the fixative for a predetermined period of time. Suitable periods of time can vary according to a variety of factors. However, under many circumstances, a suitable period of time can be approximately 6 hours. This period is generally sufficient to provide stabilization of the proteins in the collected tissue samples to substantially prevent degeneration of the collected tissue samples.

After fixation is complete, the collected tissue samples are subjected to various chemical solutions during the processing step represented by box (390). During this process, multiple tissue processing cassettes may be loaded into a basket for bulk processing. Various chemicals are then applied, which may enter each tissue processing cassette via vents similar to vents (224, 236) described above. Various chemicals may be used during this process such as alcohols of various concentration levels. For instance, when alcohol is used, moisture is removed from each collected tissue sample rendering each collected tissue sample hard in texture and generally dehydrated.

Once processing is complete, the collected tissue samples are subjected to embedding process represented by box (392). During the embedding process, the collected tissue samples are surrounded by a histological wax. In one merely exemplary embedding process, the tissue samples are removed from the tissue processing cassette and placed into a metal tray or container. Prior to placement of the tissue samples within the metal tray, the metal tray can be partially filled with an initial amount of molten wax. Once the samples are placed in the metal tray, the metal tray is then filled with molten wax. The tissue processing cassette is then placed on the top of the metal tray with the underside of the cassette facing the tissue samples. Additional molten wax is then added through the cassette to bond with wax in the metal tray. During this process, the metal tray can be placed on a cold plate or other cold surface to provide relatively quick solidification of the wax. Once solidification is complete, the collected tissue samples and cassette can be removed from the metal tray. It should be understood that once the tissue samples are prepared in this manner, the tissue samples are generally preserved for indefinite storage at room temperature.

After the embedding process is complete, thin slices of each collected tissue sample are acquired as represented by box (400). Sample sectioning may be performed using a microtome machine. Such a machine uses precision blades to slice thin samples longitudinally from each collected tissue sample. The thin sections are then placed on slides for viewing under suitable visualization means such as optical microscopes.

Once the tissue sample sections are placed on a slide, the sections are subjected to staining as represented by box (410). The portion of the collected tissue samples that remain in the tissue processing cassette are transported to storage as represented by box (420). During the staining process, various chemical compounds are applied to the tissue sample sections. Each chemical compound may be configured to react to different tissue cells. For instance, some compounds may be configured to specifically react with cancer cells, thereby staining cancer cells with a distinctive color relative to other cells. Although not represented in FIG. 7, it should be understood that in some examples the staining process can include multiple stages of staining. For instance, in some examples staining can include primary staining followed by advanced staining.

Once staining is complete, the stained sample sections are analyzed by an operator using a microscope or other visualization means as represented by box (430). Based on this analysis a diagnosis may be generated as represented by box (440).

### III. Exemplary Integrated Tissue Collection and Processing System

In some instances it may be desirable to combine certain elements of the tissue sample holder assembly (40) described above with the tissue analysis cassette (200) described above. For instance, manipulation of tissue samples generally risks degrading the quality of the tissue samples each time the tissue samples are manipulated due to the fragility of the tissue. Transferring tissue samples between elements like tissue sample tray (100) described above and tissue processing cassette (200) described above often result in at least some manipulation of the tissue samples being transferred. Thus, transferring tissue samples between various elements may be undesirable in certain circumstances because this can lead to degradation of tissue sample quality. It is therefore desirable to reduce the number of containers used to deposit tissue samples during the workflow (300) described above.

In addition to manipulation of tissue samples being generally undesirable, transferring tissue samples between different containers (e.g., tissue sample tray (100), tissue processing cassette (200)) can lead to mislabeling or tacking errors associated with tissue samples as the tissue samples progress through the workflow (300) described above. For instance, when tissue sample are transferred from tissue sample tray (100) to tissue processing cassette (200), incorrect patient information might be printed on tissue processing cassette (200). Another possibility is that an incorrect label may be placed on tissue processing cassette (200). Thus, transferring tissue samples between different containers also includes the risk of generating errors in tissue sample tracking. Accordingly, it is desirable to reduce the number of containers used in the workflow (300) described above to generally improve tissue sample integrity and reduce operator error.

Although various devices and methods are described below for reducing the number of containers used in the workflow (300) described above are described herein, it should be understood that various alternative configurations will be apparent to those of ordinary skill in the art in view of the teachings herein. For instance, some suitable alternative configurations may combine various features of one embodiment described below with various features of another alternative embodiment. Still other suitable alternative configurations may omit various features of one or more embodiments. Of course, other suitable configurations will be apparent to those of ordinary skill in the art in view of the teachings herein.

A. Exemplary Cassette Assembly

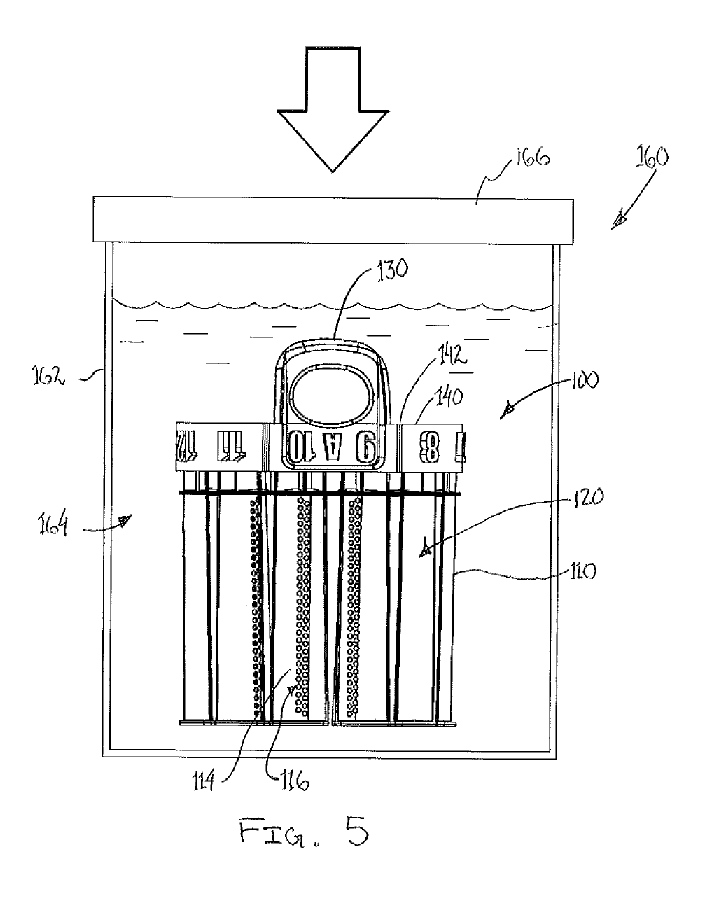

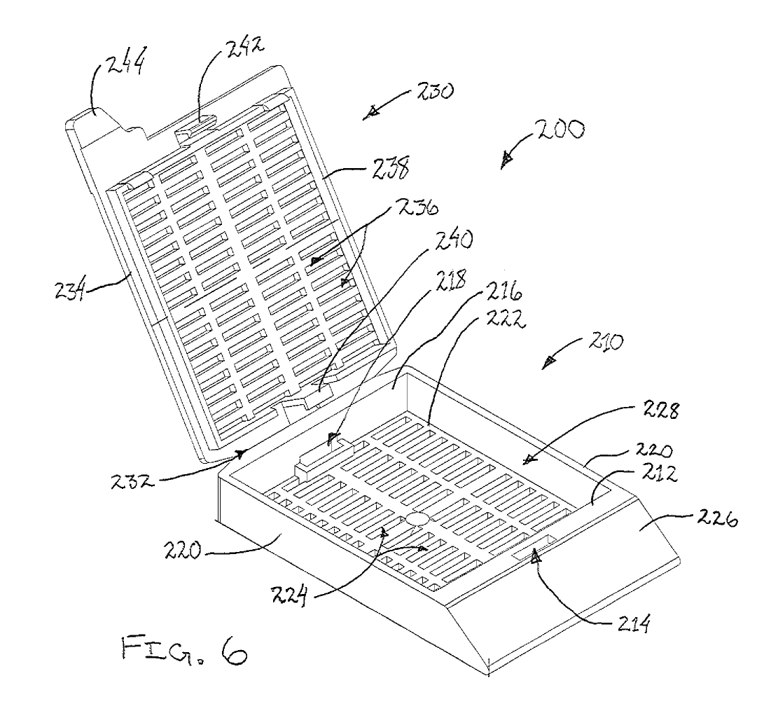

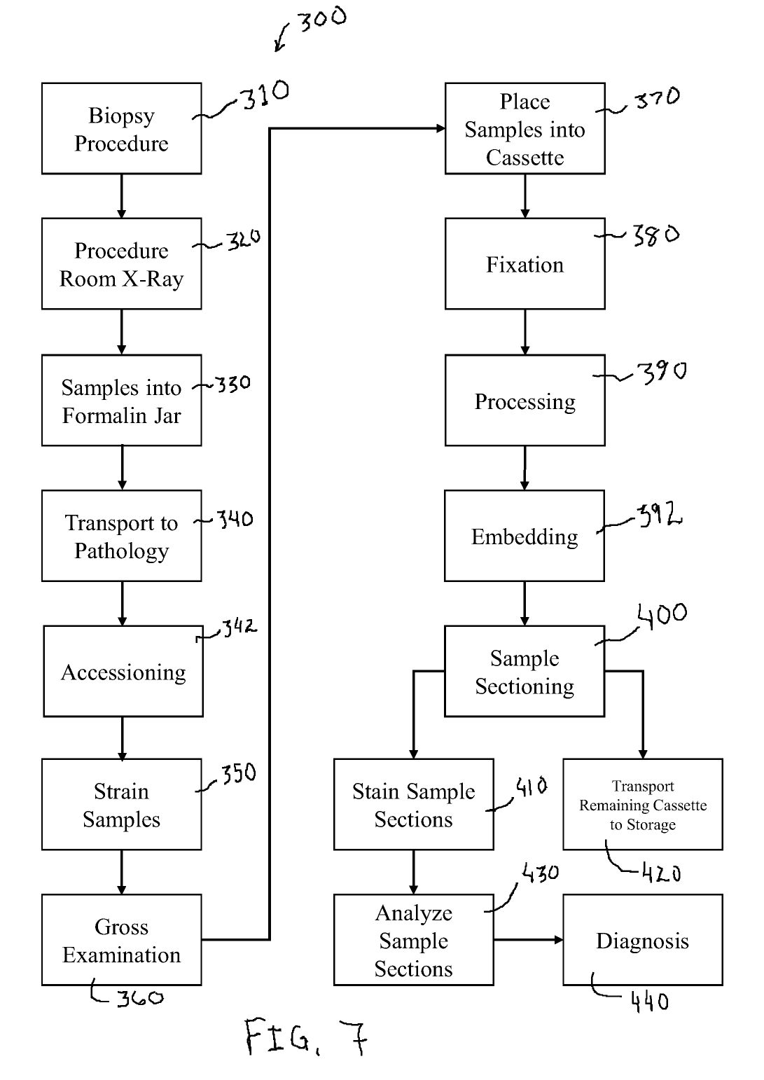

FIGS. 8-14 show and exemplary cassette assembly (500) that may be used with biopsy device (10) described above. As will be understood, cassette assembly (500) is generally configured to receive tissue samples during a biopsy procedure and then continue to contain the tissue samples after the biopsy procedure and through various sample analysis procedures. In other words, cassette assembly (500) can be used in lieu at least tissue processing cassette (200) described above. In addition, cassette assembly (500) may also be used in lieu of tissue sample tray (100), as will be described in greater detail below. However, in some uses, cassette assembly (500) may merely be supplementary to tissue sample tray (100) or other analogous features (e.g., a bulk sample basket).

As best seen in FIG. 10, cassette assembly (500) includes a cassette tray (510) and a cover (540). Cassette tray (510) comprises a distal wall (512), a proximal wall (516), a pair of sidewalls (520) extending between distal wall (512) and proximal wall (516), and a floor (524) positioned below walls (512, 516, 520). Distal wall (512) includes a plurality of openings (514) evenly spaced laterally across the face of distal wall (512). As will be described in greater detail below, each opening (514) is generally configured to receive a tissue sample. Proximal wall (516), by contrast, is solid. However, unlike distal wall (512), proximal wall (516) includes a plurality of indicia (518) on the upper surface of proximal wall (516). In the present example, indicia (518) form a plurality of unique numerical identifiers. In other examples, indicia (518) may take a variety of forms such as letters or discrete shapes or symbols.

Walls (512, 516, 520) are interconnected to form the outer perimeter of cassette tray (510). Internally, cassette tray (510) includes a plurality of inner divider walls (522) extending longitudinally from distal wall (512) to proximal wall (516). Each inner divider wall (522) is positioned parallel relative to sidewalls (520) an equal distance apart to define a plurality of discrete sample chambers (523). Each sample chamber (524) is generally configured to hold a single tissue sample severed by biopsy device (10). Although the present example includes four discrete sample chambers (523), it should be understood that in other examples any other suitable number of sample chambers (523) can be used. In such examples, it should be understood that each sample chamber (523) can be configured for receiving more than a single tissue sample as with sample chambers (523) in the present example.

Floor (524) is positioned below walls (512, 516, 520, 522). In the present example, each wall (512, 516, 520, 522) is integral with each wall. However, in other examples one or more of each wall (512, 516, 520, 522) can be separate from floor (524) and attached with adhesive or some form of mechanical fastening. Floor (524) includes a plurality of vents (526). Vents (526) are generally configured to promote the flow of fluid through floor (524), yet maintain tissue samples within each sample chamber (523). To facilitate this configuration, vents (526) have a narrow rectangular form. In other examples, vents (526) can be configured with a variety of alternative shapes such as round, oval-shaped, square, and/or etc. Although vents (526) in the present example are arranged to uniformly occupy the entire surface of floor (524), it should be understood that in other examples vents (526) can be arranged in a variety of other ways. For instance, vents (526) can be isolated to a specific region or multiple regions of floor (524). Of course, other alternative arrangements for vents (526) will be apparent to those of ordinary skill in the art in view of the teachings herein.

Floor (524) is opposite to an open space above each sample chamber (523). Thus, the upper portion of cassette tray (510) is generally open. Because of this, tissue samples may be deposited into each sample chamber (523) through openings (514) in distal wall (512) or through the open upper portion of cassette tray (510). As will be described in greater detail below, tissue samples are generally contained within each sample chamber (523) once cassette tray (510) is received within cover (540).

Cassette tray (510) further includes a labeling portion (528) protruding proximally from proximal wall (516). Labeling portion (528) generally defines a triangular or wedge shape that provides a flat smooth surface for printing or otherwise adhering a label to the surface of labeling portion (528). As similarly described above with respect to labeling surface (226) of tissue processing cassette (200), labeling portion (528) is generally configured to provide readily accessible patient information to an operator to aid with tracking of tissue samples as they progress through the biopsy and sample analysis procedure.

Unlike labeling surface (226) described above with respect to tissue processing cassette (200), at least a portion of labeling portion (528) is generally oversized relative to the height of sidewalls (520) or the lateral length of proximal wall (516). This feature generally provides a blocking or sealing feature for cassette tray (510) to promote the flow of fluid through cassette tray (510). As will be described in greater detail below, cassette tray (510) is generally insertable into cover (540) or other components. When inserted into cover (540) or other suitable components, labeling portion (528) blocks at least a portion of cover (540) and/or other components to force fluid flow through vents (526) rather than other features of cassette tray (510).

As best seen in FIG. 12, cassette tray (510) further comprises a plurality of detents (530, 532) disposed on the underside of floor (524). As can be seen, cassette tray (510) comprises a pair of distal detents (530) and a pair of proximal detents (532). Distal detents (530) are positioned approximately adjacent to distal wall (512), while proximal detents (532) are positioned approximately adjacent to proximal wall (516). As will be described in greater detail below, each pair of detents (530, 532) is positioned to provide temporary or selective locking of cassette tray (510) at various positions relative to cover (540) when cassette tray (510) is inserted into cover (540). Although detents (530, 532) are shown as having a generally rectangular shape with rounded corners, it should be understood that various alternative shapes may be used in other examples. For instance, detents (530, 532) can be hemispherical, oval-shaped, triangular, and/or etc.

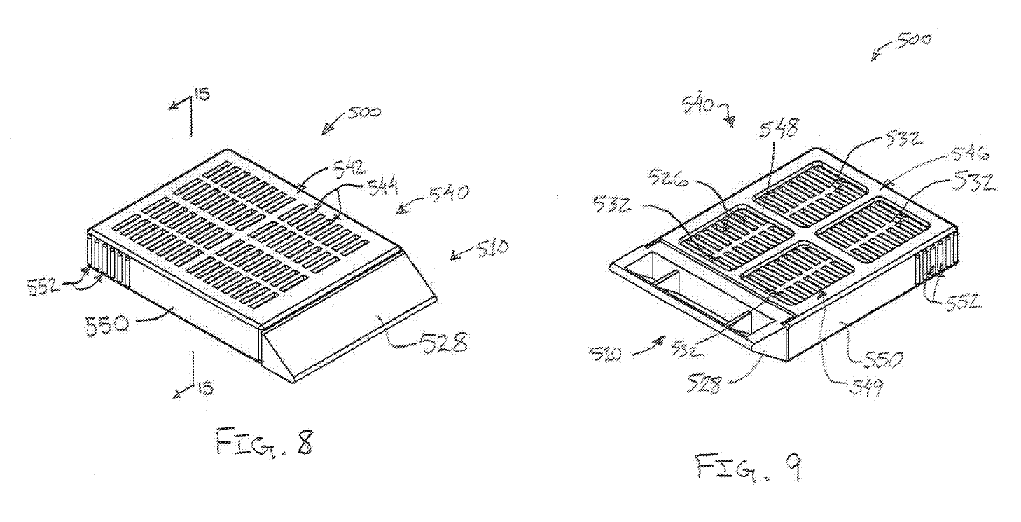

FIGS. 13 and 14 show cover (540) in greater detail. As can be seen, cover (540) comprises a filter portion (542), a support portion (546), and a plurality of walls (550, 554) extending between the filter portion (542) and the support portion (546). Filter portion (542) is similar to floor (524) described above in that filter portion (542) includes a plurality of vents (544) arranged in an array about the surface of filter portion (542). Vents (544) are generally configured to promote the flow of fluid through filter portion (542), yet maintain tissue samples within each sample chamber (523) of cassette tray (510) when cassette tray (510) is inserted into cover (540). To facilitate this configuration, vents (544) have a narrow rectangular form. In other examples, vents (544) can be configured with a variety of alternative shapes such as round, oval-shaped, square, and/or etc. Although vents (544) in the present example are arranged to uniformly occupy the entire surface of filter portion (542), it should be understood that in other examples vents (544) can be arranged in a variety of other ways. For instance, vents (544) can be isolated to a specific region or multiple regions of filter portion (542). Of course, other alternative arrangements for vents (544) will be apparent to those of ordinary skill in the art in view of the teachings herein.

Unlike filter portion (542), support portion (546) omits structures similar to vents (544). Instead, support portion (546) includes a support structure (548) defining a plurality of open spaces (549). As will be understood, support portion (546) is generally adjacent to floor (524) of cassette tray (510) when cassette tray (510) is inserted into cover (540). Thus, including structures similar to vents (544) is not entirely necessary due to the presence of vents (526) in floor (524) of cassette tray (510). However, it should be understood that in some examples support portion (546) may include structures similar to vents (544).

Support structure (548) forms a generally cross-shaped pattern in support portion (546). This structure is generally configured to provide rigidity to cover (540) and is further configured to hold cassette tray (510) within cover (540) when cassette tray (510) is disposed within cover (540). Although support structure (548) forms a generally cross-shaped pattern in the present example, it should be understood that in other examples support structure (548) can take on a variety of other forms. For instance, in some examples support structure (548) can have a lath-shaped structure. In other examples, support structure (548) can have a lattice shaped structure. In still other examples, support structure (548) can be formed of a plurality of concentric circles, or any other configuration as will be apparent to those of ordinary skill in the art in view of the teachings herein.

Support structure (548) is further configured to interact with detents (530, 532) of cassette tray (510). As will be described in greater detail below, cassette tray (510) is generally insertable into cover (540) at a plurality of discrete positions relative to cover (540). During insertion, detents (530, 532) of cassette tray (510) interact with support structure (548) to bias cassette tray (510) towards each discrete position. Due to the cross-shaped pattern of support structure (548), support structure (548) provides three discrete positions of cassette tray (510) relative to cover (540). Of course, in other examples where support structure (548) defines a different shape, support structure (548) can provide more or less discrete positions for cassette tray (510) relative to cover (540).

With the cross-shaped pattern of support portion (546), support portion (546) defines four total open spaces (549). Open spaces (549) are generally configured to promote fluid flow through cover (540) between vents (544) of filter portion (542) and open spaces (549). As will be described in greater detail below, this permits fluid to flow through cassette tray (510) when cassette tray (510) is disposed within cover (540).

As described above, cover (540) includes a plurality of walls (550, 554) extending between filter portion (542) and support portion (546). Walls (550, 554) include a pair of sidewalls (550) and a distal wall (554). Sidewalls (550) and distal wall (554) are both solid to generally promote rigidity of cover (540). Each sidewall (550) includes a plurality of grips (552), which promote manipulation of cover (540) by an operator. Walls (550, 554) together with filter portion (542) and support portion (546) together are configured to define an enclosure for cassette tray (510) that holds tissue samples within cassette tray (510), while permitting fluid to flow through cassette tray (510).

Opposite distal wall (554), filter portion (542), support portion (546), and sidewalls (550) define a proximal opening (558). Proximal opening (558) is generally configured to receive at least a portion of cassette tray (510) such that cassette tray (510) may be inserted into cover (540). Although proximal opening (558) is shown in the present example as having a generally rectangular shape, it should be understood that proximal opening (558) is generally a function of the shape of cover (540) and cassette tray (510). Thus, in examples where cassette tray (510) and/or cover (540) take on different shapes, proximal opening (558) may also be correspondingly different.

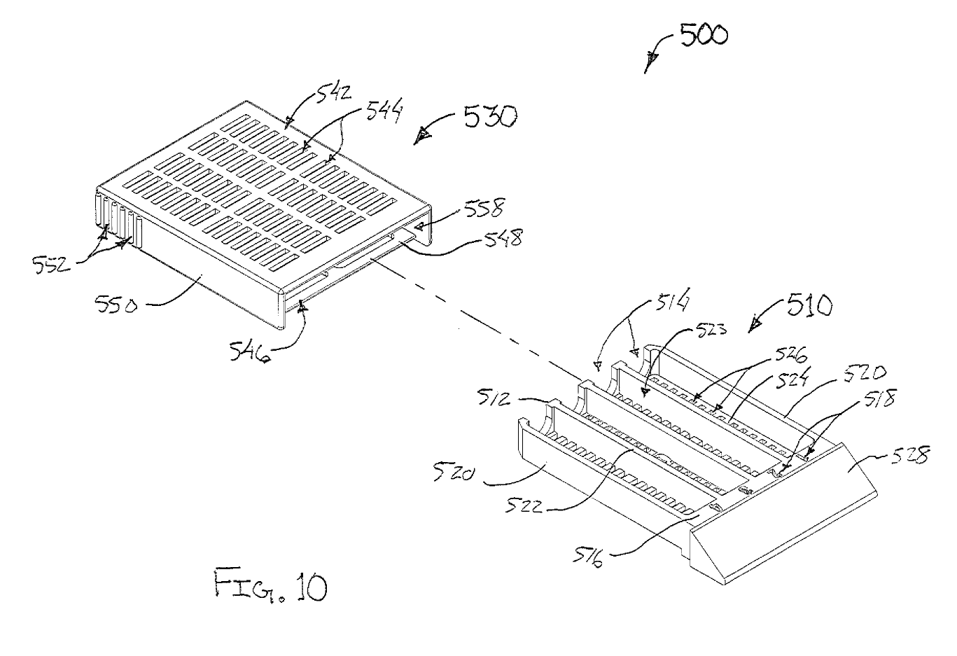

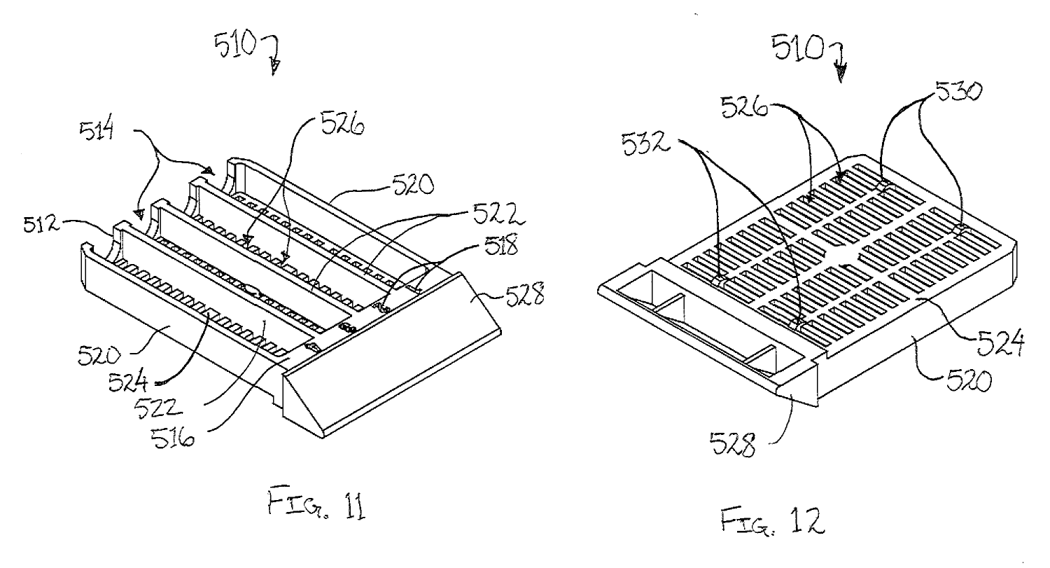

FIGS. 15A-15C show an exemplary insertion of cassette tray (510) into cover (540). As will be described in greater detail below, insertion of cassette tray (510) into cover (540) generally occurs during a biopsy procedure after tissue samples have been collected by biopsy device (10) and inserted into cassette tray (510). As can be seen in FIG. 15A, the distal end of cassette tray (510) is initially inserted into proximal opening (558) of cover (540). As cassette tray (510) is inserted into proximal opening (558) of cover (540), distal detents (530) engage support structure (548) of support portion (546). Further insertion of cassette tray (510) into cover (540) causes distal detents (530) to flex over support structure (548) before releasing into the open spaces (549) that are oriented proximally on support portion (546).

Once distal detents (530) are disposed within the open spaces (549) that are oriented proximally on support portion (546) as shown in FIG. 15A, cassette tray (510) is generally removably secured within cover (540). In this context, “removably secured” refers to how cassette tray (510) is generally restricted from being pulled proximally out of cover (540). However, it should be understood that cassette tray (510) may still be pulled proximally out of cover (540) if a sufficient amount of force is applied to flex distal detents (530) upwardly onto support structure (548). At the same time, it should be understood that cassette tray (510) remains freely translatable in the distal direction such that cassette tray (510) can be advanced further into cover (540). In the position shown in FIG. 15A, cassette tray (510) can be optionally used by an operator while partially disposed within cover (540). By way of example only, this may be desirable for positioning or repositioning tissue samples within cassette tray (510).

Once cassette tray (510) is initially inserted into cover (540), an operator can insert cassette tray (510) further into cover (540) in the proximal direction towards the position shown in FIG. 15B. Cassette tray (510) is freely insertable in the proximal direction until distal detents (530) again engage support structure (548). Once distal detents (530) are engaged with support structure (548), an operator can apply a force to cassette tray (510) or cover (540) to flex distal detents (530) onto support structure (548) (or to flex support structure (548) out of the way of distal detents (530)).

Once distal detents (530) are clear of support structure (548), distal detents (530) will flex back to their original position and into the open spaces (549) oriented distally on cover (540). Once distal detents (530) are disposed within the open spaces (549) that are oriented distally on support portion (546) as shown in FIG. 15B, cassette tray (510) is generally removably secured within cover (540). Similarly to the context above, “removably secured” here refers to how cassette tray (510) is generally restricted from being pulled proximally out of cover (540). However, it should be understood that cassette tray (510) may still be pulled proximally out of cover (540) if a sufficient amount of force is applied to flex distal detents (530) upwardly onto support structure (548). At the same time, it should be understood that cassette tray (510) remains freely translatable in the distal direction such that cassette tray (510) can be advanced further into cover (540). In the position shown in FIG. 15B, cassette tray (510) can be optionally used by an operator while partially disposed within cover (540). By way of example only, this may be desirable for positioning or repositioning tissue samples within cassette tray (510).

Once cassette tray (510) is inserted into cover (540) to the position shown in FIG. 15B, an operator may desire to insert cassette tray (510) fully into cover (540). To insert cassette tray (510) fully into cover (540), an operator may move cassette tray (510) distally relative to cover (540) towards the position shown in FIG. 15C. As cassette tray (510) is moved distally relative to cover (540), proximal detents (532) will engage support structure (548) of cover. At this point, an operator can apply a force to either cassette tray (510) or cover (540) that is sufficient to flex proximal detents (532) upwardly and onto support structure (548) (or flex support structure (548) out of the way of proximal detents (532)). Cassette tray (510) can then proceed further distally until proximal detents (532) flex downwardly to their original position and into the open spaces (549) oriented proximally on cover (540) as shown in FIG. 15C.

Once cassette tray (510) is positioned relative to cover (540) as shown in FIG. 15C, further distal movement of cassette tray (510) is prevented by engagement between distal wall (512) of cassette tray (510) and distal wall (554) of cover (540). In addition, as described above, labeling portion (528) is generally oversized relative to the dimensions of proximal wall (516) of cassette tray (510) and sidewalls (520) of cassette tray (510). Accordingly, labeling portion (528) can also act to stop further distal movement of cassette tray (510) by engagement between labeling portion (528) and support portion (546), sidewalls (550), and filter portion (542) of cover (540). In addition, it should be understood that in some contexts filter portion (542) can also act as a seal to seal proximal opening (558) of cover (540) relative to the exterior of cover (540). In such circumstances, this sealing can act to force fluid through vents (526, 544) rather than proximal opening (558).

B. Exemplary Alternative Tissue Sample Holder Assembly

In some examples it may be desirable to use cassette tray (510) in connection with biopsy device (10) such that tissue samples are collected directly into cassette tray (510) rather than into a structure similar to tissue sample tray (100) described above. Because cassette tray (510) includes a generally rigid structure, it should be understood that cassette tray (510) is generally not insertable directly into rotatable member (44) described above. Instead, it may be desirable to replace tissue sample holder assembly (40) with an alternative tissue sample holder assembly to facilitate use of cassette tray (510) directly with biopsy device (10). As described above, tissue sample holder assembly (40) is generally configured to be completely removable from probe assembly (20) of biopsy device (10). Thus, a suitable alternative tissue sample holder assembly may be used in lieu of tissue holder assembly (40), provided certain vacuum and tissue sample collection couplings remain consistent between the suitable alternative tissue sample holder assembly and tissue sample holder assembly (40).

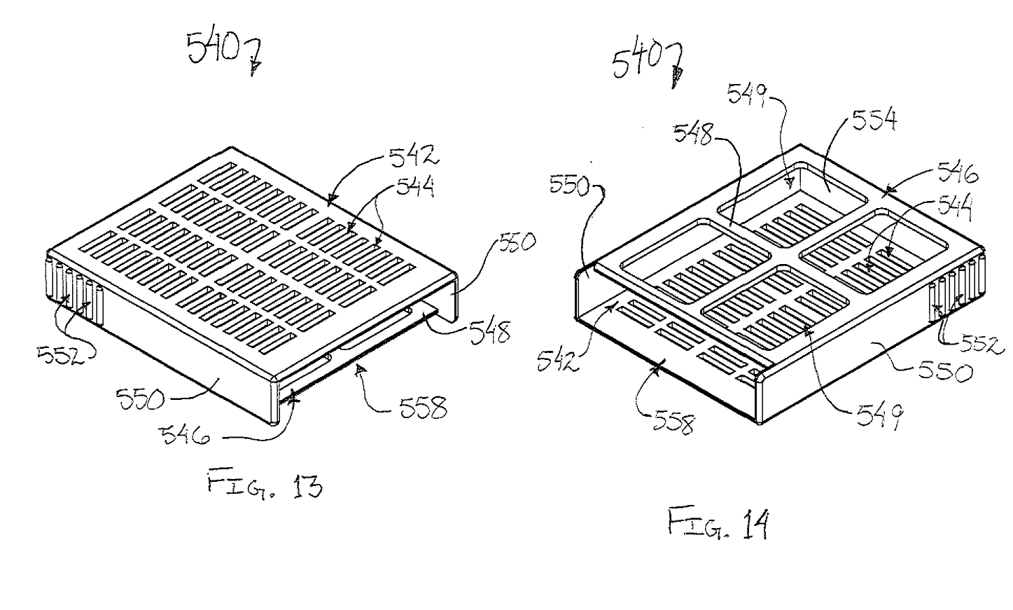

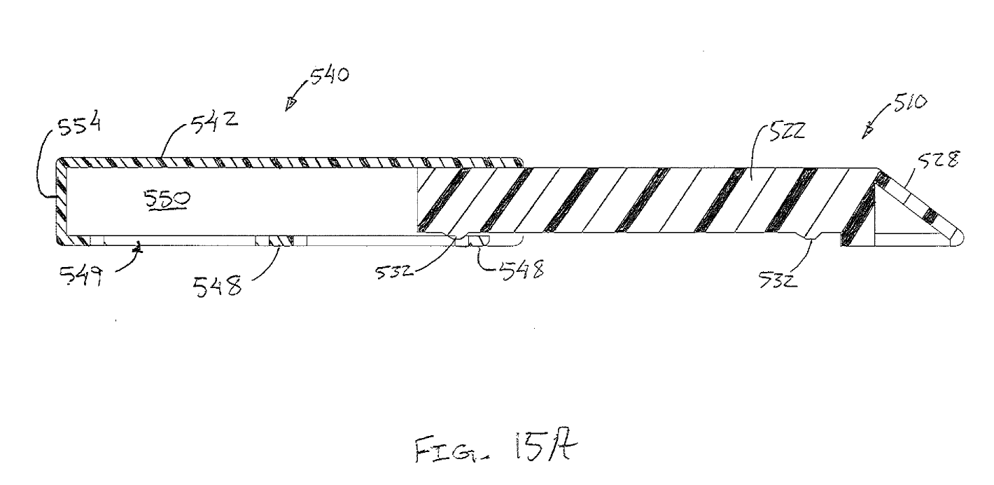

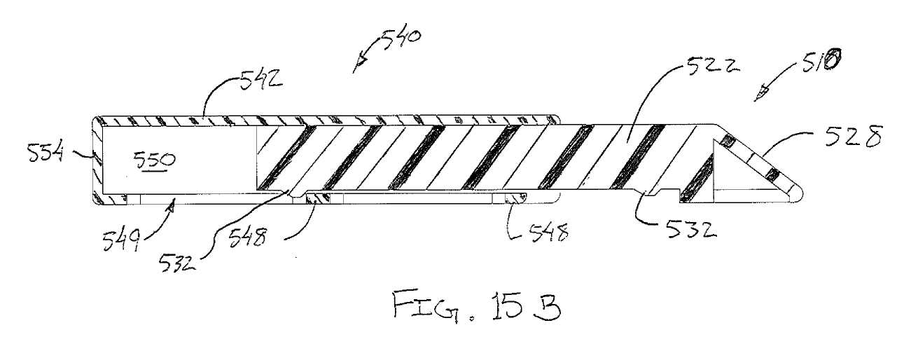

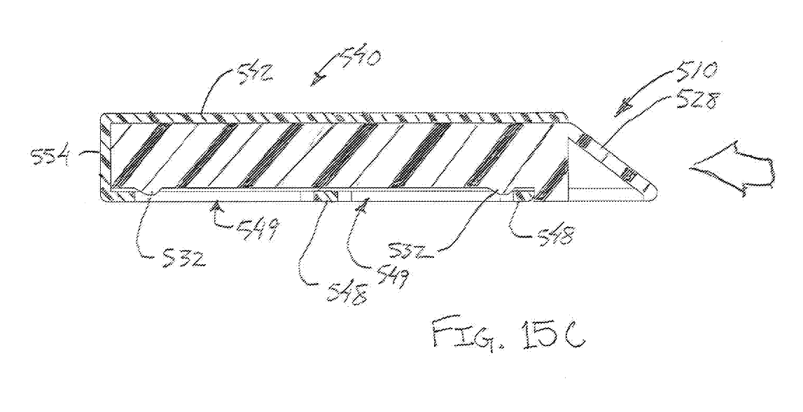

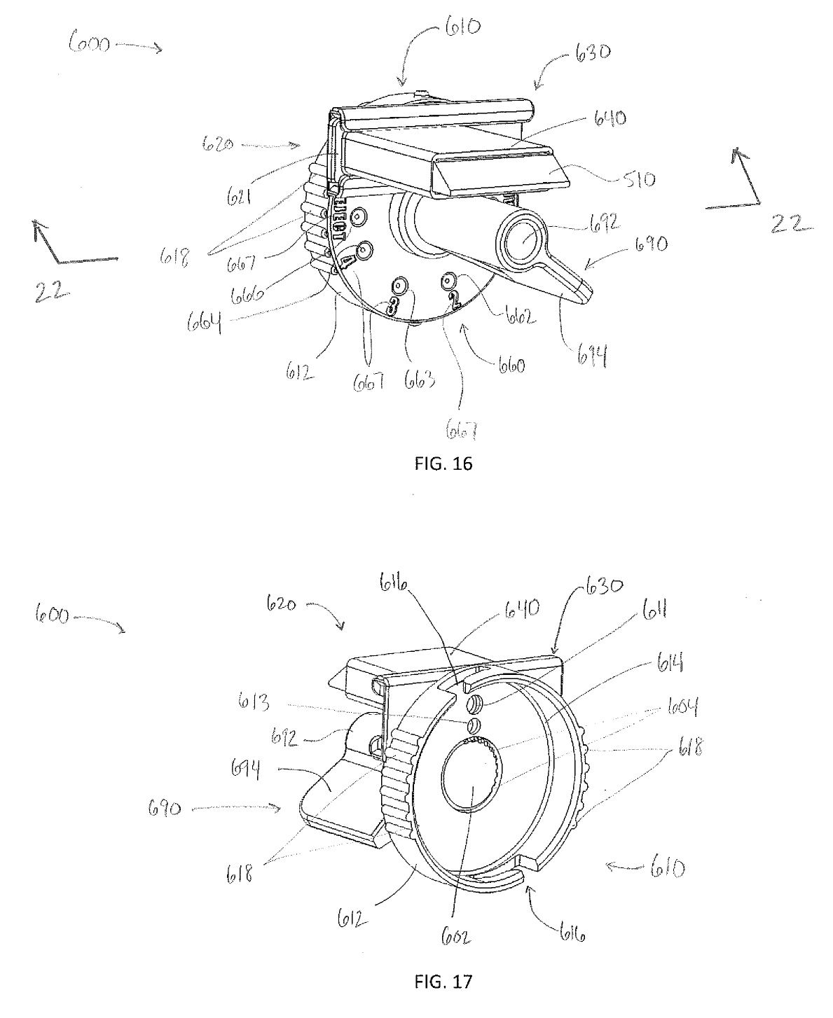

FIGS. 16-20 show an exemplary alternative tissue sample holder assembly (600) that may be used with biopsy device (10) in lieu of tissue sample holder assembly (40) described above. As best seen in FIG. 16, tissue sample holder assembly (600) comprises a coupler (610), a slidable member (620), a track (630), and an indexing mechanism (660). Slidable member (620) is generally configured to receive cassette tray (510) and position cassette tray (510) relative to probe assembly (20) to thereby collect a tissue sample within each sample chamber (523) of cassette tray (510). Slidable member (620) comprises a rectangular base (621) and a manifold (640) or cassette holder, with rectangular base (621) serving as a distal wall of manifold (640). Base (621) extends along the distal perimeter of manifold (640) such that manifold (640) extends proximally from base (621). The rectangular shape of base (621) is generally configured for receipt within track (630) such that at least a portion of manifold (640) abuts track (630). Base (621) is sized and configured to slidably translate manifold (640) along track (630).

Although not shown, in some versions tissue sample holder assembly (600) may further include a sensor manifold positioned adjacent to track (630) that is configured to receive a diagnostic imaging sensor therein. In this instance, with the sensor received within the sensor manifold, the sensor is effectively positioned adjacent to manifold (640) when slidable member (620) is received within track (630) such that the sensor is located proximate to cassette tray (510). The sensor may be operable to convert and transmit data digitally in conjunction with an imaging device to thereby communicate energy beams therebetween and generate images of any contents contained within cassette tray (510), such as extracted tissue samples. Including a sensor manifold along tissue sample holder assembly (600) that receives a sensor may be beneficial to quickly examine recently biopsied tissue specimens through certain imaging modalities to thereby quickly analyze and assess the tissue properties. Examples and methods of a sensor manifold and corresponding imaging system that comprises a sensor and imaging device are described in U.S. application Ser. No. [Attorney Docket No. LEI 20061-SO-US], entitled “Tissue Collection and Processing Cassette with Applied Imaging,” filed on even date herewith.

Coupler (610) comprises a generally ring-shaped body (612) with a sealing edge (614), a pair of bayonet connectors (616), and a plurality of grips (618). Sealing edge (614) is configured to engage at least a portion of probe assembly (20) to seal tissue sample holder assembly (600) to biopsy device (10). In addition, sealing edge (614) is configured to permit translation of slidable member (620) relative to coupler (610) and probe assembly (20) while maintaining a sealed connection between tissue sample holder assembly (600) and biopsy device (10). As will be described in greater detail below, this translation permits cassette tray (510) to be moved relative to probe assembly (20) so that a single tissue sample can be collected within each sample chamber (523) of cassette tray (510).

As best seen in FIG. 17, bayonet connectors (616) are configured to receive a pair of bayonet pins (not shown) of probe assembly (20) to selectively couple coupler (610) to probe assembly (20). Thus, bayonet connectors (616) and the bayonet pins of probe assembly (20) form a standard bayonet coupling assembly to selectively secure coupler (610) to probe assembly (20). In this configuration, ring-shaped body (612) is generally rotatable relative to probe assembly (20) to lock and unlock coupler (610) relative to probe assembly (20). To assist an operator with rotation of ring-shaped body (612), coupler (610) includes grips (618) to enhance grip of ring-shaped body (612) during locking and unlocking. Although the present example uses a bayonet coupling to secure coupler (610) to probe assembly (20), it should be understood that in other examples various alternative coupling features can be used as will be apparent to those of ordinary skill in the art in view of the teachings herein.

Coupler (610) further includes an access port (611) and a vacuum port (613) defined between sealing edge (614) to a proximal end through track (630) (see FIG. 19). As will be described in greater detail below, access port (611) is sized and shaped to receive tissue samples therethrough and to correspond with an access opening (622, 623, 624, 625) in manifold (640). Access port (611) is configured to provide fluid communication between probe assembly (20) and tissue sample holder assembly (600) such that a tissue sample extracted by the cutter of biopsy device (10) may be communicated to cassette tray (510) contained in manifold (640). As will also be described in greater detail below, vacuum port (613) is sized and shaped to provide a vacuum through manifold (640) and to correspond with a vacuum opening (626, 627, 628, 629) of manifold (640). Vacuum port (613) is configured to provide vacuum communication between probe assembly (20) and tissue sample holder assembly (600) such that a tissue sample extracted by the cutter of biopsy device (10) may be effectively transferred into cassette tray (510) contained in manifold (640).

As shown in FIG. 17, indexing mechanism (660) comprises a gear (602) positioned at a center of coupler (610) and an actuator (690). Gear (602) includes a plurality of teeth (604) extending along the perimeter of gear (602). Gear (602) is fixedly secured to actuator (690) such that rotation of actuator (690) causes the simultaneously rotation of gear (602). Although not shown, it should be understood that a top portion of gear (602) extends into a bottom flange (634) of track (630) such that plurality of teeth (604) extend into track (630). As will be described in greater detail below, plurality of teeth (604) are sized and shaped to mesh with a plurality of teeth (645) of base (621).

Actuator (690) includes a rotatable fastener (692) and arm (694), with arm (694) extending laterally from rotatable fastener (692). By way of example only, actuator (690) may be in the form of a handle, knob, lever, or other various suitable actuators as will be apparent to those of ordinary skill in the art. Rotatable fastener (692) is configured to rotatably couple arm (694) to coupler (610) such that arm (694) is configured to rotate about rotatable fastener (692) relative to coupler (610). Gear (602) is coupled to rotatable fastener (692) of actuator (690) such that gear (602) is in communication with arm (694) through rotatable fastener (692). In other words, rotation of arm (694) about rotatable fastener (692) provides for the simultaneous rotation of gear (602) within coupler (610).

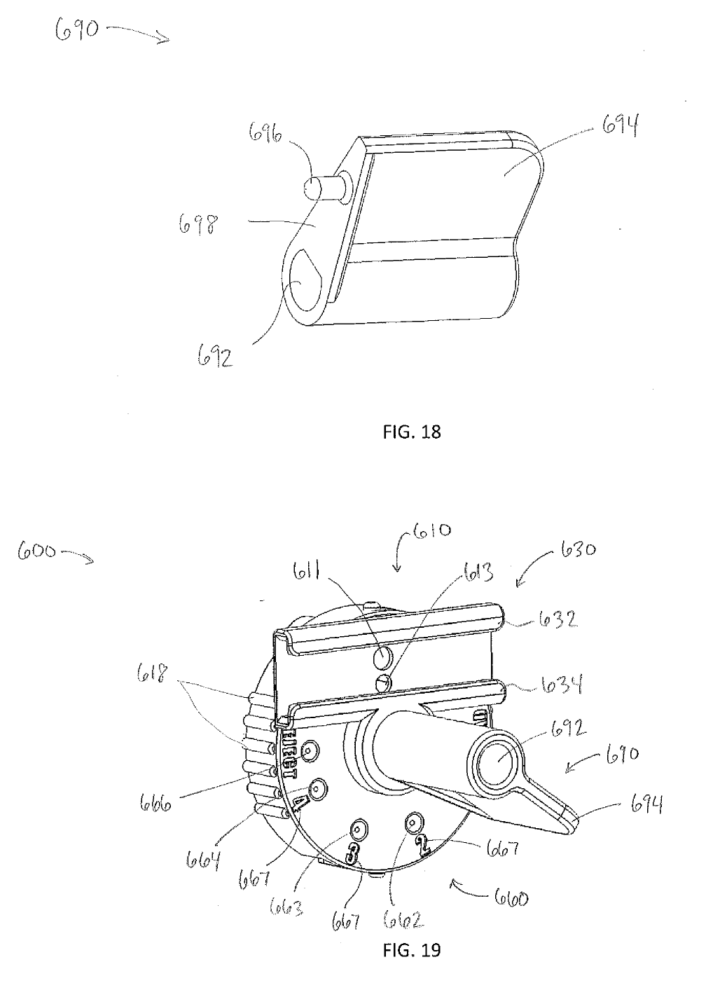

As shown in FIG. 18, actuator (690) further comprises a flexible detent (696) along an inner surface (698) of arm (694) adjacent to coupler (610) such that the flexible detent (696) of actuator (690) abuts against coupler (610). By way of example only, flexible detent (696) of actuator (690) may be in the form of a compressible pin that is retractable into arm (694) and similarly extendable outwardly from arm (694). As will be described in greater detail below, flexible detent (696) is configured to couple with various portions of coupler (610) to thereby releasably fix actuator (690) to various rotatable orientations relative to coupler (610).

Indexing mechanism (660) further comprises multiple receiving apertures or slots (661, 662, 663, 664, 665, 666) and corresponding labels (667) for each receiving slot (661, 662, 663, 664, 665, 666), as best seen in FIG. 16. Receiving slots (661, 662, 663, 664, 665, 666) are circular indentations or depressions spaced along coupler (610) at predetermined locations that extend inwardly into coupler (610) at a depth that corresponds with the length of flexible detent (696). Although not shown, it should be understood that receiving slots (661, 662, 663, 664, 665, 666) may comprise various other shapes, profiles, and depths that are suitable to receive flexible detent (696) therein as will be apparent to those of ordinary skill in the art. In this instance, indexing mechanism (660) includes a first slot (661), a second slot (662), a third slot (663), a fourth slot (664), a loading slot (665) and an ejecting slot (666) with a corresponding label (667) for each that indicates the identity of that particular slot (661, 662, 663, 664, 665, 666), respectively.

Slots (661, 662, 663, 664, 665, 666) are recessed from the surface of coupler (610) and are configured to receive the flexible detent (696) of actuator (690) therein such that slots (661, 662, 663, 664, 665, 666) are operable to couple actuator (690) at a respective orientation about rotatable fastener (692) when the flexible detent (696) is rotated to the particular slot (661, 662, 663, 664, 665, 666) and coupled therein with that slot (661, 662, 663, 664, 665, 666). The flexible detent (696) of actuator (690) couples with slots (661, 662, 663, 664, 665, 666) by extending outwardly from arm (694) when aligning with a particular slot (661, 662, 663, 664, 665, 666) as arm (694) rotates about rotatable fastener (692). It should be understood that although slots (661, 662, 663, 664, 665, 666) are shown as having a specific position on coupler (610) and a particular angle and/or geometry relative to coupler (610), the particular configuration of slots (661, 662, 663, 664, 665, 666) may be adjusted based on a number of considerations such as the positioning of slidable member (620), the number of plurality of teeth (645) on slidable member (620), the number of plurality of teeth (604) on gear (602), the number and/or size of each access opening (622, 623, 624, 625) relative to cassette tray (510), and/or etc.

FIG. 19 shows tissue sample holder assembly (600) with slidable member (620) removed from track (630). As shown, access port (611) and vacuum port (613) extend through coupler (610) and into track (630). Track (630) includes a top flange (632) and a bottom flange (634) extending across along the length of track (630). Flanges (632, 634) are sized and shaped to slidably receive and hold base (621) of slidable member (620) to thereby couple manifold (640) to coupler (610). In other words, top flange (632) is spaced apart from bottom flange (634) in accordance with the length of base (621) of slidable member (620) such that track (630) is configured to securely grasp slidable member (620) between flanges (632, 634), as shown in FIG. 16.

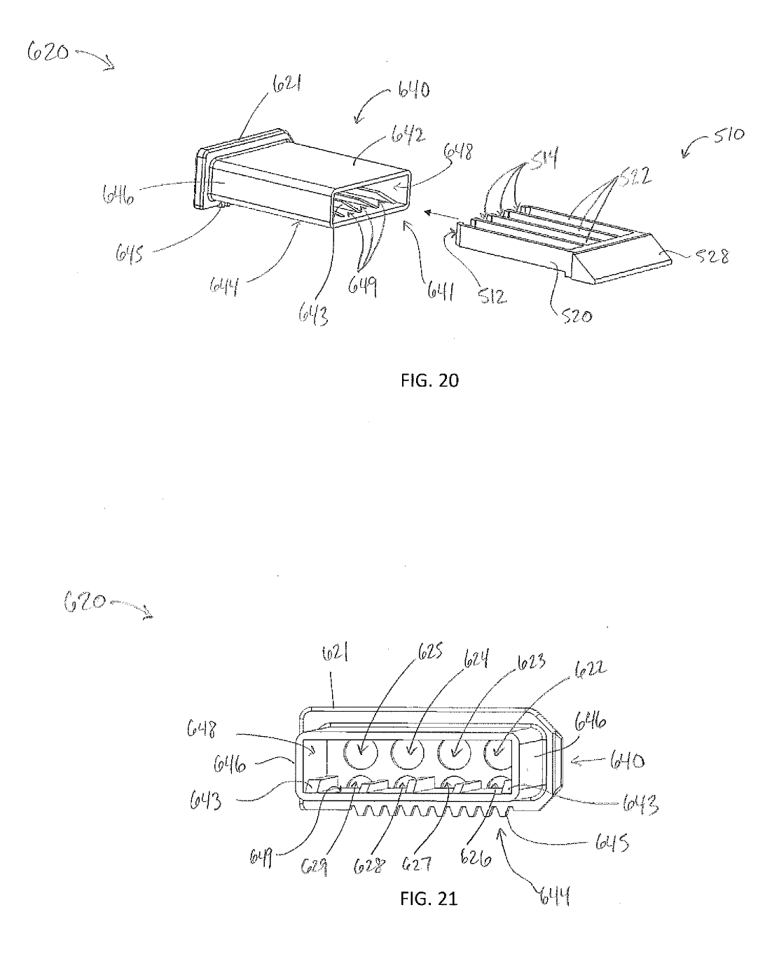

As seen in FIG. 20, base (621) has a length that is relatively larger than the length of manifold (640). Manifold (640) comprises an upper wall (642), a lower wall (644), and a pair of sidewalls (646) extending between the lower wall (644) and the upper wall (642). Walls (642, 644, 646) together define a generally rectangular box that is configured to receive cassette tray (510). Walls (642, 644, 646) further define an inner chamber (648) that is large enough to accommodate cassette tray (510), while also providing fluid flow through manifold (640). In other words, inner chamber (648) is sized and shaped to receive and hold cassette tray (510) therein. As will be described in greater detail below, inner chamber (648) is generally configured to receive tissue samples axially relative to the longitudinal axis of slidable member (620) and direct tissue samples into cassette tray (510).

Access to the inner chamber (648) of manifold (640) is provided through a chamber opening (641) that is positioned relatively opposite of base (621). Manifold (640) further includes multiple rails (643) along an inner surface of lower wall (644) such that rails (643) extend inwardly into inner chamber (648) and towards upper wall (642) to define multiple channels (649). In other words, inner chamber (648) includes multiple channels (649) extending along an inner surface of lower wall (644) that are defined between two adjacent rails (643). Rails (643) are sized and shaped to slidably receive and hold cassette tray (510) within inner chamber (648) when slidably inserted through chamber opening (641) and into manifold (640). As will be described in greater detail below, rails (643) are configured to position cassette tray (510) within manifold (640) at a height that corresponds with access port (611) of coupler (610) when slidable member (620) is slidably coupled to track (630).

Base (621) includes a plurality of teeth (645) along a bottom portion of base (621) such that plurality of teeth (645) are aligned along a similar plane as lower wall (644) of manifold (640). As best seen in FIG. 21, plurality of teeth (645) extend along a substantial length of the bottom portion of base (621). In the present example, each tooth (645) of plurality of teeth (645) is irregularly shaped and spaced apart from one another to thereby define a recess in between each tooth (645). Although not shown, it should be understood that teeth (645) may comprise various other suitable shapes, profiles, and configurations along the bottom portion of base (621). As merely an illustrative example, teeth (645) may be rectangularly shaped, circular, squared, or any other suitable shape as will be apparent to those of ordinary skill in the art. As will be described in greater detail below, plurality of teeth (645) are configured to engage with at least a portion of indexing mechanism (660) to thereby translate manifold (640) along track (630). In particular, plurality of teeth (645) are sized and shaped to associate with corresponding teeth (604) of gear (602) such that each tooth (604) is operable to fit in between a respective recess in between and mesh with each tooth (645) of plurality of teeth (645). Correspondingly, teeth (645) are sized and shaped to associate with teeth (604) of gear (602) such that each tooth (645) is operable to fit in between and mesh with a respective recess in between each tooth (604) of gear (602).

Base (621) further includes multiple access openings (622, 623, 624, 625) and vacuum openings (626, 627, 628, 629) positioned along the lateral length of base (621) at the distal end of manifold (640). In particular, access openings (622, 623, 624, 625) are positioned directly on top of vacuum openings (626, 627, 628, 629) relative to base (621). As can be seen, base (621) includes first access opening (622) and first vacuum opening (626) that are positioned along base (621) to correspond to first slot (661), and more generally, a first state and/or position of tissue sample holder assembly (600). Second access opening (623) and second vacuum opening (627) are positioned along base (621) to correspond to second slot (662), and more generally, a second state and/or position of tissue sample holder assembly (600); and a third access opening (624) and a third vacuum opening (628) are positioned along base (621) to correspond to third slot (663), and more generally, a third state and/or position of tissue sample holder assembly (600). Lastly, fourth access opening (625) and fourth vacuum opening (629) are positioned along base (621) to correspond to fourth slot (664), and more generally, a fourth state and/or position of tissue sample holder assembly (600). As will be described in greater detail below, slidable member (620) is configured such that rotating actuator (690) from one slot (661, 662, 663, 664) to another simultaneously realigns another access opening (622, 623, 624, 625) and vacuum opening (626, 627, 628, 629) in communication with access port (611) and vacuum port (613) respectively.

Access openings (622, 623, 624, 625) provide access to inner chamber (648) at a distal end of manifold (640) opposite of chamber opening (641). Access openings (622, 623, 624, 625) and vacuum openings (626, 627, 628, 629) are aligned along base (621) to correspond to a particular channel (649) such that each pair of access and vacuum openings (622, 623, 624, 625, 626, 627, 628, 629) is in direct communication with the channel (649) in inner chamber (648) that is positioned in alignment with that particular pair of openings (622, 623, 624, 625, 626, 627, 628, 629).

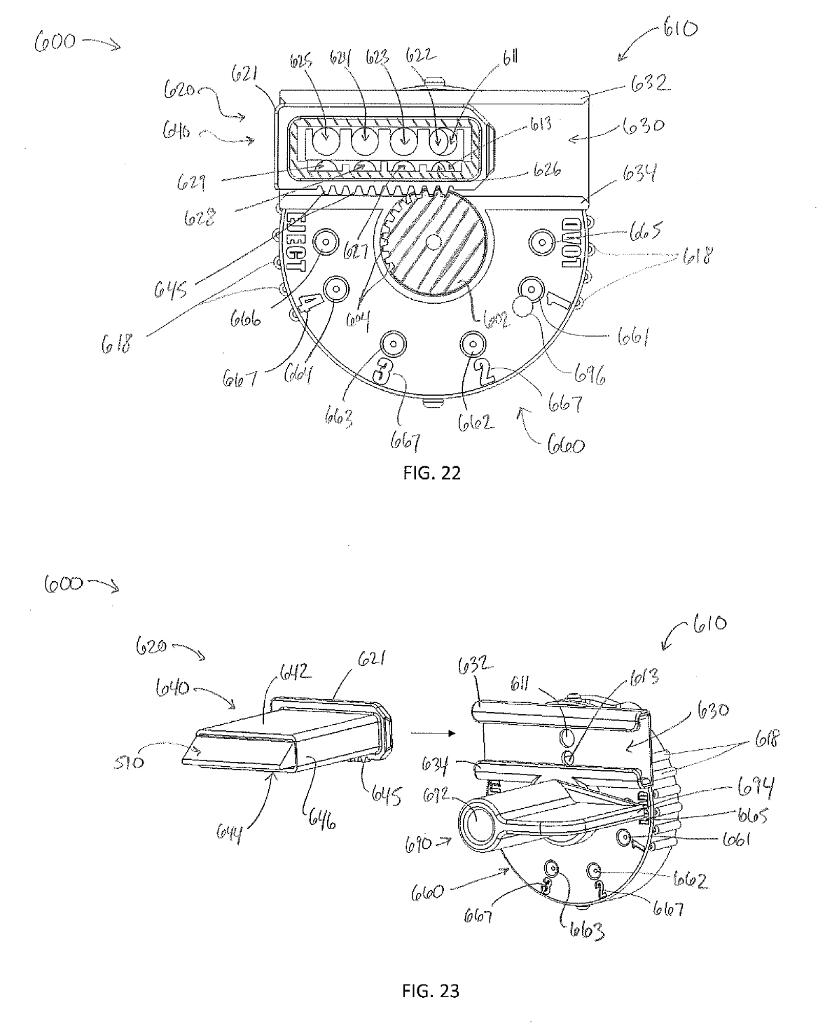

FIG. 22 shows slidable member (620) translated along track (630) to align a particular access opening (622, 623, 624, 625) with access port (611) and a particular vacuum opening (626, 627, 628, 629) with vacuum port (613). In the example shown in FIG. 22, first access opening (622) and first vacuum opening (626) are partially aligned with ports (611, 613), respectively. The schematic representation of the location of the flexible detent (696) of actuator (690) indicates that arm (694) is rotated to an orientation beyond first slot (661) and directed toward second slot (662) such that first openings (622, 626) are slidably translated such that communication with ports (611, 613), respectively, are partially obstructed.

When a particular access opening (622, 623, 624, 625) and vacuum opening (626, 627, 628, 629) is aligned with ports (611, 613), respectively, that access opening (622, 623, 624, 625) can be used to gain access to the biopsy site through the cutter of needle (22). In this configuration, the aligned access opening (622, 623, 624, 625) communicates with access port (611) while the aligned vacuum opening (626, 627, 628, 629) is in communication with the vacuum port (613). With cassette tray (510) received within inner chamber (548) of manifold (540), translation of slidable member (620) along track (630) is operable to place a particular sample chamber (523) of cassette tray (510) into communication with the cutter of needle (22), as seen in FIG. 22. Slidable member (620) slides along track (630) from the meshing engagement of plurality of teeth (645) of manifold (640) with plurality of teeth (604) of gear (602). As will be described in greater detail below, plurality of teeth (604) rotate clockwise about gear (602) as actuator (690) rotates clockwise from one slot (661, 662, 663, 664, 665) to a subsequent slot (661, 662, 663, 664, 666), respectively.

As further seen in FIG. 22, rails (643) are sized to align chambers (523) of cassette tray (510) with access openings (622, 623, 624, 625) when cassette tray (510) is received in inner chamber (648) of manifold (640). Rails (643) are further sized and configured to provide ample space in each channel (649) of inner chamber (648) for vacuum openings (626, 627, 628, 629) to allow for communication between inner chamber (648) and vacuum port (613). In other words, within inner chamber (652), the plurality of channels (649) defined by the plurality of rails (643) are formed by rails (643) extending upwardly from lower wall (644) partially into inner chamber (648) such that support for cassette tray (510) is created and a vacuum chamber is created. Each channel (645) is in communication with a corresponding vacuum opening (626, 627, 628, 629) to communicate vacuum from probe assembly (20) and into cassette tray (510). With each vacuum opening (626, 627, 628, 629) being generally associated with a corresponding access opening (622, 623, 624, 625), only a single vacuum opening (626, 627, 628, 629) is in communication with a vacuum source when the particular corresponding access opening (622, 623, 624, 625) is in communication with the cutter of needle (22). As will be described in greater detail below, this configuration generally promotes the flow of vacuum into a given vacuum opening (626, 627, 628, 629), into inner chamber (648) (and through cassette tray (510)) and out of a corresponding access opening (622, 623, 624, 625).

FIGS. 23-25D show an exemplary use of tissue sample holder assembly (600) to collect tissue samples within cassette tray (510). Once cassette tray (510) is inserted into manifold (640) of slidable member (620), as shown in FIG. 20, slidable member (620) is advanced toward coupler (610) as shown in FIG. 23. Although not shown, it should be understood that at this stage tissue sample holder assembly (600) is generally already coupled to probe assembly (20) via coupler (610) in lieu of tissue sample holder assembly (40). However, it should be understood that in other uses, cassette tray (510) may be first inserted into manifold (640) and then tissue sample holder assembly (600) may be attached to probe assembly (20). Regardless of whether tissue sample holder assembly (600) is attached to probe assembly (20), cassette tray (510) may be inserted into manifold (640) by inserting distal wall (512) of cassette tray (510) into the chamber opening (641) of manifold (640), as seen in FIG. 20. Chamber opening (641) of manifold (640) provides an adequately sized opening into inner chamber (648) for cassette tray (510) to be inserted through. Accordingly, cassette tray (510) can be simply inserted into manifold (640). This engagement provides sealing of cassette tray (510) relative to the exterior of manifold (640). Although not shown, it should be understood that in some examples either manifold (640) or cassette tray (510) can include additional sealing features such as rubber gaskets to aid in the sealing of cassette tray (510) relative to the exterior of manifold (640). In other examples, sealing is provided by a compression fit between walls (642, 644, 646) of manifold (640) and cassette tray (510).

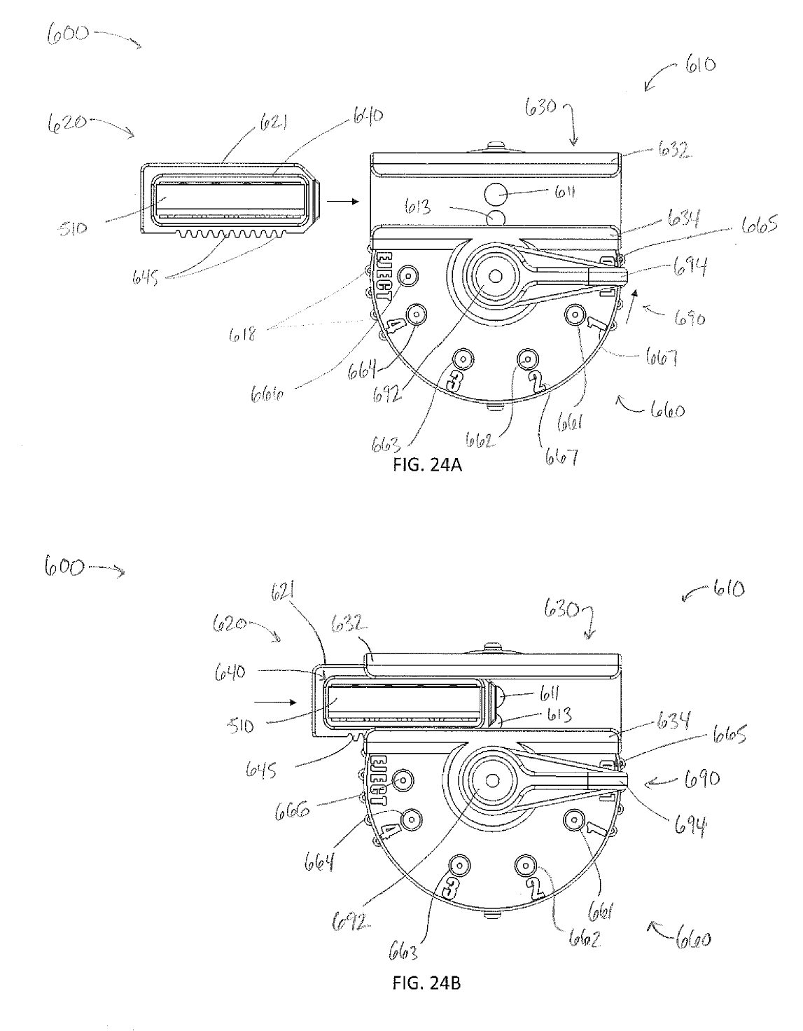

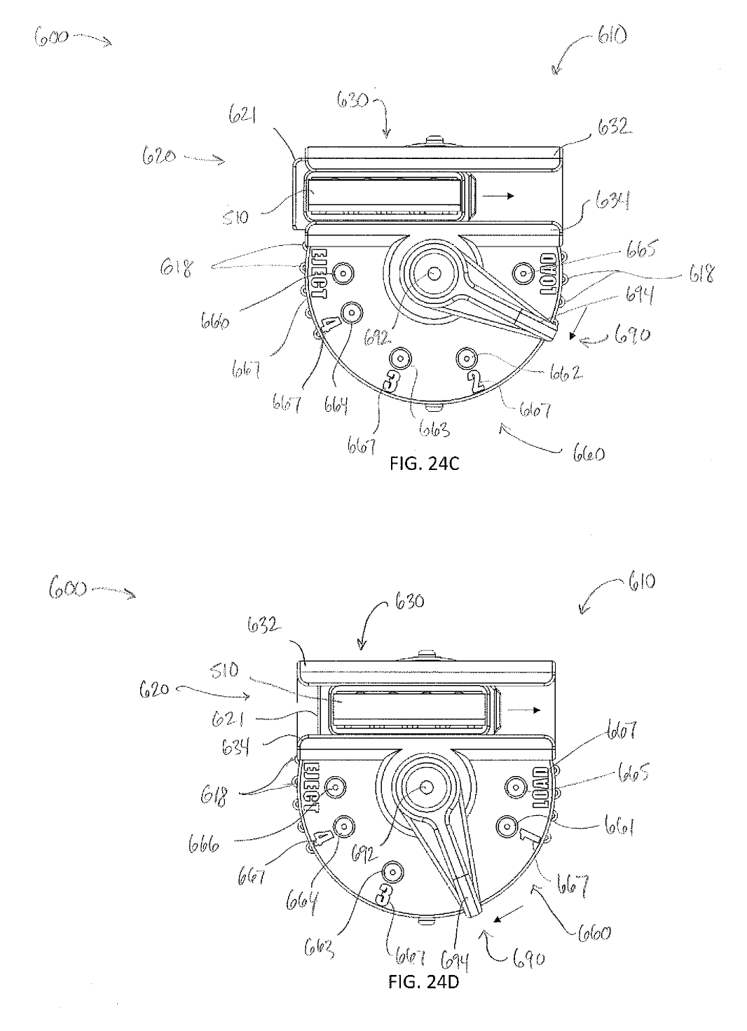

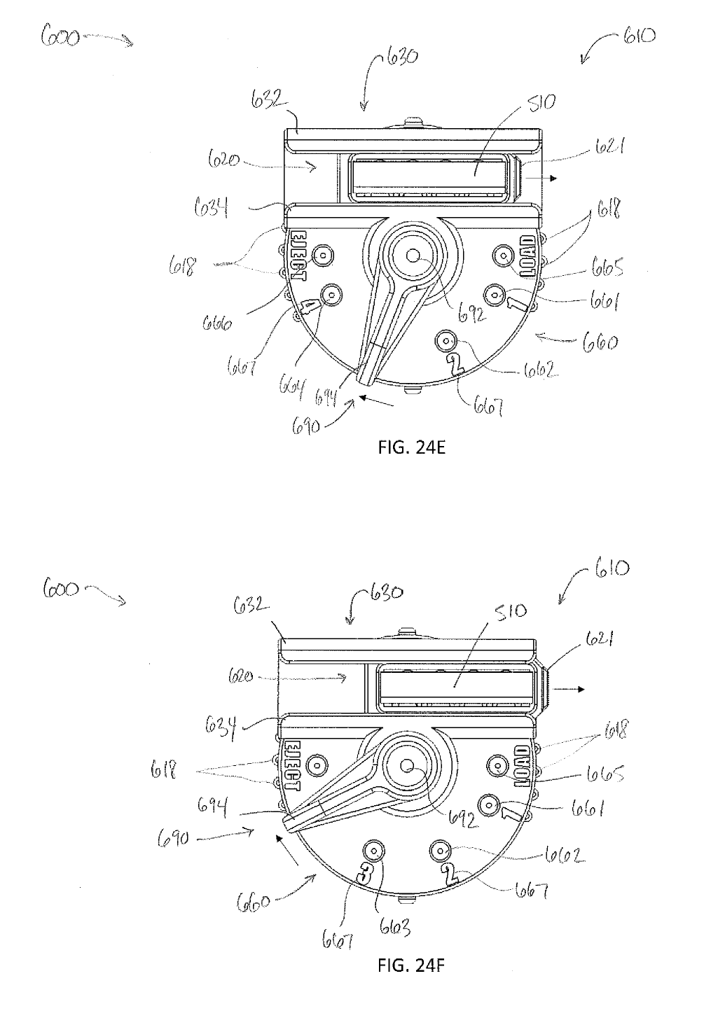

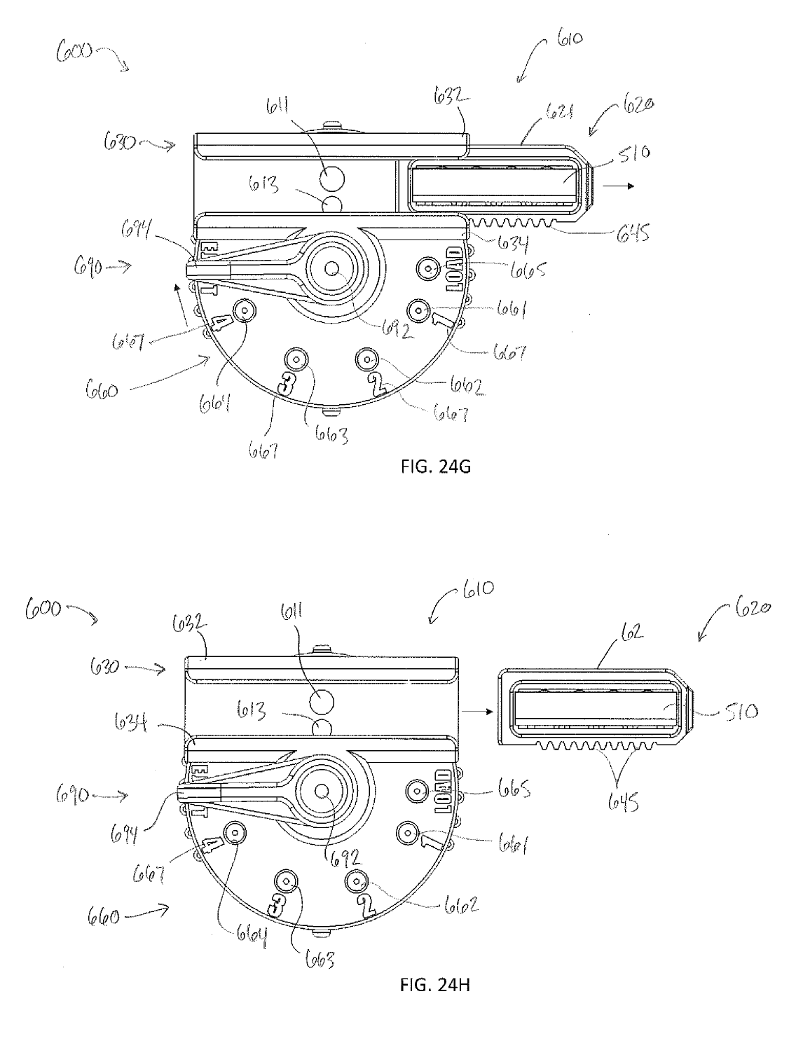

With cassette tray (510) received within manifold (640), slidable member (620) is advanced towards track (630) to engage slidable member (620) to coupler (610), as seen in FIG. 23. In this instance, actuator (690) is rotated to a loading state and/or position where arm (694) is oriented over loading slot (665) such that the flexible detent (696) of actuator (690) couples with loading slot (665), as further seen in FIG. 24A. With actuator (690) coupled to the loading slot (665), slidable member (620) is able to be received within track (630) buy slidably translating base (621) between flanges (632, 634) with plurality of teeth (645) positioned adjacent to bottom flange (634). As seen in FIG. 24B, slidable member (620) may be advanced through track (630) until plurality of teeth (645) encounter plurality of teeth (604) of gear (602). In this instance, plurality of teeth (645) mesh with plurality of teeth (604) but slidable member (620) is not able to be advanced further with tissue sample holder assembly (600) still in the loading position. With slidable member (620) maintained in this intermediate position, first access opening (622) and first vacuum opening (626) are not in direct alignment with access port (611) and vacuum port (613), respectively, as seen in FIG. 24B.

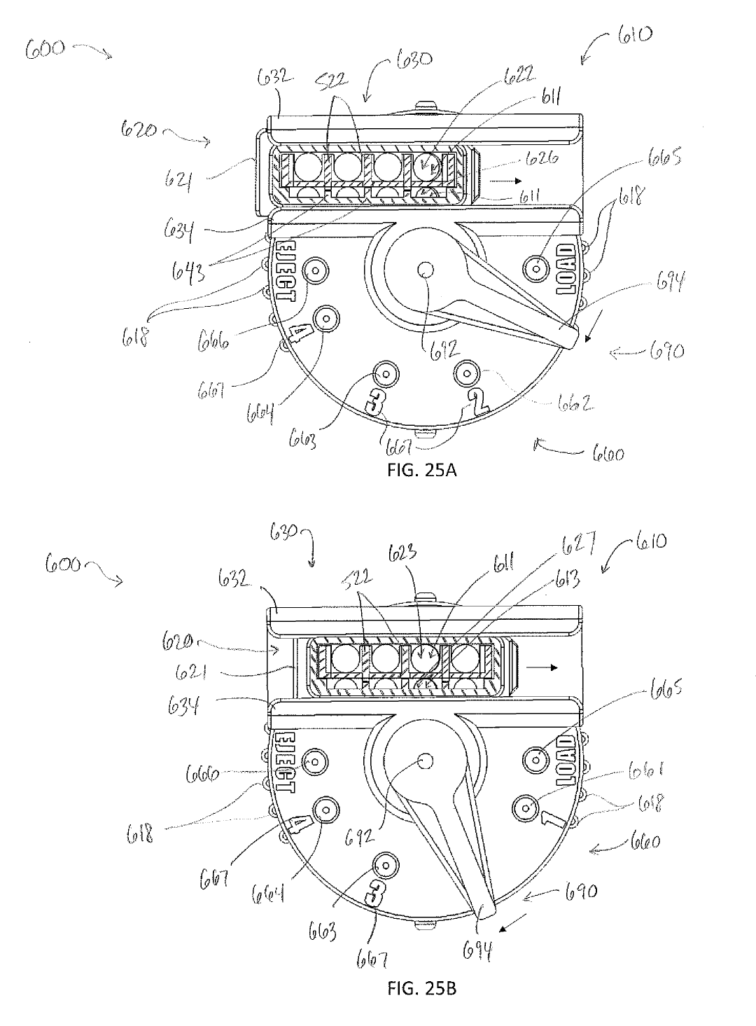

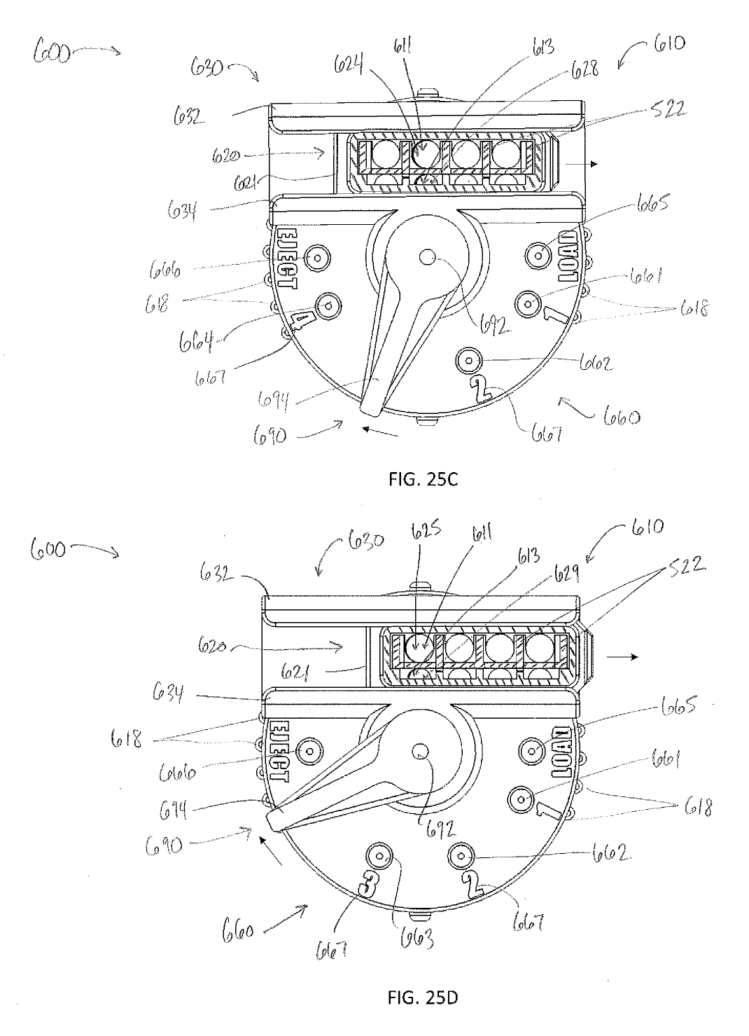

FIG. 24C shows the rotation of actuator (690) from the loading state/position to the first state/position where a predetermined force is applied to arm (694) to decouple flexible detent (696) from loading slot (665) and thereby couple the flexible detent (696) to first slot (661). The clockwise rotation of arm (694) from the loading state to the first state causes the simultaneous clockwise rotation of gear (602) which results in plurality of teeth (604) pivoting with gear (602). Due to the meshed engagement between plurality of teeth (604) and plurality of teeth (645), slidable member (620) is translated along track (630) in a lateral direction as shown in FIG. 24C. In this instance, as best seen in FIG. 25, first access opening (622) and first vacuum opening (626) of manifold (640) become aligned with access port (611) and vacuum port (613), respectively, to thereby establish fluid communication between the cutter of probe assembly (20) and the particular chamber (523) of cassette tray (510) that is aligned with openings (622, 626). With tissue sample holder assembly (600) now in the first position, vacuum enters manifold (640) through vacuum opening (626) that is in communication with corresponding vacuum port (613) of coupler (610). Next, vacuum travels through the corresponding channel (645) and upwardly through vents (526) of cassette tray (510). Vacuum then travels through a corresponding sample chamber (523) of cassette tray (510) and out of manifold (640) through access opening (622) which is positioned in communication with the cutter of needle (22). Vacuum is then used to pull a tissue sample through the cutter of needle (22) and into the corresponding sample chamber (526) of cassette tray (510).

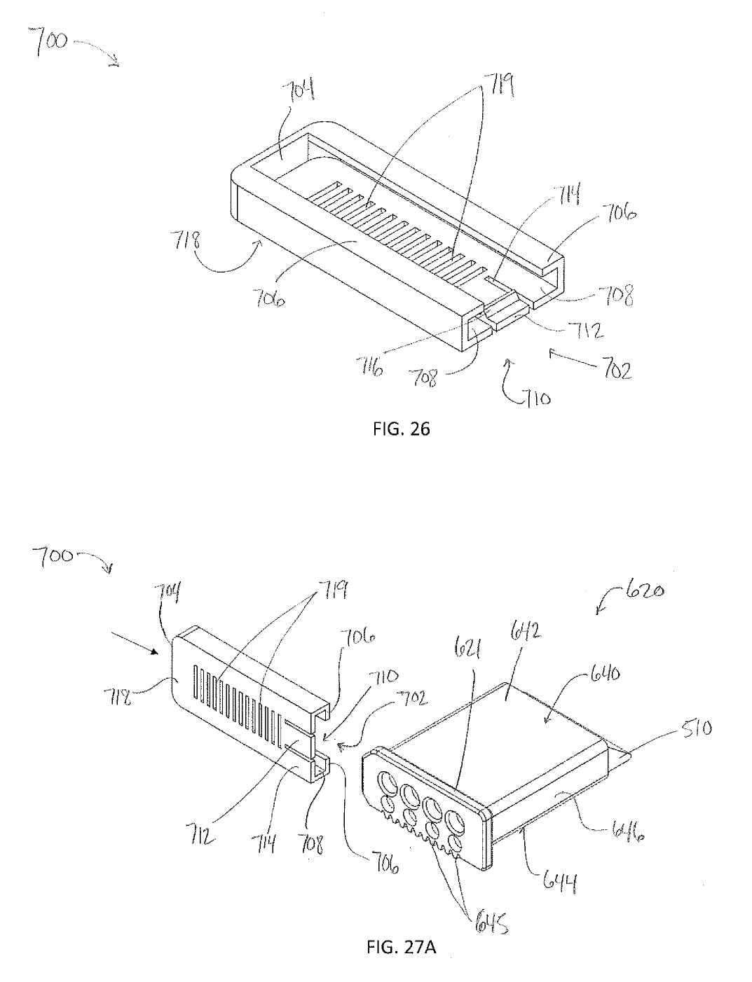

Once the desired number of tissue samples have been deposited into the corresponding sample chamber (523) that is aligned with first access opening (622), a predetermined force is applied to arm (698) to decouple the flexible detent (696) from first slot (661) and thereby rotate actuator (690) until the flexible detent (696) couples with second slot (662). This rotation indexes the next successive sample chamber (523) of cassette tray (510) to receive a tissue sample therein. The clockwise rotation of arm (694) from the first state to the second state causes the simultaneous clockwise rotation of gear (602) which results in plurality of teeth (604) pivoting with gear (602). Due to the meshed engagement between plurality of teeth (604) and plurality of teeth (645), slidable member (620) is translated along track (630) in a lateral direction as shown in FIG. 24D. In this instance, as best seen in FIG. 26, second access opening (623) and second vacuum opening (627) of manifold (640) become aligned with access port (611) and vacuum port (613), respectively, to thereby establish fluid communication between the cutter of probe assembly (20) and the particular chamber (523) of cassette tray (510) that is aligned with openings (623, 627). With tissue sample holder assembly (600) now in the second position, vacuum enters manifold (640) through vacuum opening (627) that is in communication with corresponding vacuum port (613) of coupler (610). Next, vacuum travels through the corresponding channel (645) and upwardly through vents (526) of cassette tray (510). Vacuum then travels through a corresponding sample chamber (523) of cassette tray (510) and out of manifold (640) through access opening (623) which is positioned in communication with the cutter of needle (22). Vacuum is then used to pull a tissue sample through the cutter of needle (22) and into the corresponding sample chamber (526) of cassette tray (510).

It should be understood that actuator (690) may be similarly rotated in a clockwise direction as described above to transition tissue sample holder assembly (600) to a third state and fourth state, respectively, where the flexible detent (696) of actuator (690) is effectively decoupled and recoupled to the subsequent third slot (663) and fourth slot (664), respectively, as shown in FIGS. 24E-24F. In those instances, slidable member (620) translates along track (630) as similarly described above such that third openings (624, 628) and fourth openings (625, 629) align with ports (611, 613), respectively and sequentially, to thereby establish fluid and vacuum communication with subsequent sample chambers (523) of cassette tray (510), as shown in FIGS. 25C-25D.

With the desired number of tissue samples having been deposited into the corresponding sample chamber (523) that is aligned with fourth access opening (625), a final predetermined force is applied to arm (698) to decouple the flexible detent (696) from fourth slot (664) and thereby rotate actuator (690) until the flexible detent (696) couples with ejecting slot (666). The clockwise rotation of arm (694) from the fourth state to the ejecting state causes the simultaneous clockwise rotation of gear (602) which results in plurality of teeth (604) pivoting with gear (602). Due to the meshed engagement between plurality of teeth (604) and plurality of teeth (645), slidable member (620) is translated along track (630) in a lateral direction until plurality of teeth (645) of manifold (640) is released from the meshed engagement with plurality of teeth (604), as shown in FIG. 24D. In this instance, fourth access opening (625) and fourth vacuum opening (629) of manifold (640) are no longer aligned with access port (611) and vacuum port (613), respectively, to thereby terminate fluid communication between the cutter of probe assembly (20) and the particular chamber (523) of cassette tray (510) that is aligned with openings (625, 629). With tissue sample holder assembly (600) now in the ejecting position, slidable member (620) is freely translatable along track (630) independent of the position and/or rotation of actuator (690) such that an operator may grasp manifold (640) to laterally slide slidable member (620) along track (630) until slidable member (620) is completely removed from flanges (632, 634), as shown in FIG. 24H.

Although tissue sample holder assembly (600) is described above as being used to collect a tissue sample in each sample chamber (523) of cassette tray (510), it should be understood that in some uses it may be desirable to only collect samples into one or more specific sample chambers (523) of cassette tray (510). Accordingly, in some uses slidable member (620) may be translated along track (630) to skip some access openings (622, 623, 624, 625) and proceed directly to indexing one or more specific access openings (622, 623, 624, 625) that are in fluid communication with particular sample chambers (523) of cassette tray (510). This is achieved by continuing to rotate actuator (690) to the particular slot (661, 662, 663, 664) that is associated with the one or more sample chambers (523) of cassette tray (510) that an operator desires to deposit tissue samples in. It should be understood that the flexible detent (696) will still couple with each slot (661, 662, 663, 664) despite the desire to rotate actuator (690) to a particular slot (661, 662, 663, 664) along coupler (610). In other words, an operator will still be required to overcome the releasable engagement of the flexible detent (696) with each slot (661, 662, 663, 664) by applying a predetermined force onto arm (694) to thereby decouple the flexible detent (696) from the current slot (661, 662, 663, 664) until the flexible detent (696) becomes secured with the particular slot (661, 662, 663, 664) that an operator desires to transition tissue sample holder assembly (600) to.

Once sample chambers (523) of cassette tray (510) are filled with a tissue sample as desired by an operator, an operator may next desire to perform certain analysis on the collected tissue samples. To perform analysis on the collected tissue samples, an operator first removes cassette tray (510) from manifold (640) of slidable member (620). At this stage, cassette tray (510) may be manipulated for a visual inspection of each tissue sample. In addition, cassette tray (510) may be placed in a procedure room x-ray unit to perform a preliminary analysis of the tissue samples. If an operator is not satisfied with the results at this stage, undesirable tissue samples may be discarded and the same cassette tray (510) may be inserted back into manifold (640) of slidable member (620) for collection of addition tissue samples. Alternatively, an entirely new cassette tray (510) may be placed into manifold (640) of slidable member (620) for collection of additional tissue samples.

Alternatively, an analysis of the collected tissue samples deposited within sample chambers (523) of cassette tray (510) may be performed while cassette tray (510) is maintained within manifold (640) of slidable member (620). In this instance, with slidable member (620) disengaged from track (630) of tissue sample holder assembly (600) and housing cassette tray (510) within inner chamber (648), the assembly of slidable member (620) and cassette tray (510) is placed in a procedure room x-ray unit to perform the preliminary analysis of the tissue samples deposited in chambers (523). In the present example, walls (642, 644) of manifold (640) are formed of a material that is configured to permit x-ray imaging of the contents in inner chamber (648) through manifold (640). In other words, manifold (640) is configured to allow for adequate imaging of any tissue samples contained on cassette tray (510) when cassette tray (510) is positioned within manifold (640) such that an operator is not required to remove cassette tray (510) from manifold (640) prior to performing the analysis of the deposited tissue samples in chambers (523).

Once tissue sample are collected to the satisfaction of an operator, the operator may desire to transport tissue samples to a pathology laboratory. At this stage, an operator may mark or place a label onto cassette tray (510) to ensure chain of custody through the workflow. Alternatively, in some uses, a label may already be included on cassette tray (510) at this stage. For instance, in some uses a label may be included on cassette tray (510) at the beginning of the biopsy procedure before collecting any tissue samples. Alternatively, in some uses a label on cassette tray (510) may be prelabeled with a bar code, QR code, or another computer readable medium. Where such computer readable mediums are used, the label may be scanned as various stages to associate the computer readable medium with the patient. This may include multiple scans throughout the procedure such as before the biopsy procedure, after collection of tissue samples, after procedure room x-ray, and/or etc.

Once chain of custody has been established using a label on cassette tray (510), cassette tray (510) may be inserted into cover (540) as shown in FIGS. 15A-15C. The combination of cassette tray (510) and cover (540) may then be inserted into jar (160) described above. As described above, jar (160) may be filled with a fluid such as formalin to preserve the collected tissue samples during transport and/or storage. Although cassette tray (510) is described herein as being used with the same jar (160) described above, it should be understood that other alternative jars or containers may be used for transport and/or storage of cassette tray (510). For instance, in some examples jar (160) may be replaced with a container of a variety of shapes and sizes. In other examples, cover (540) itself may be used to transport cassette tray (510). Of course, in such examples structures of cover (540) such as vents (544) and/or open spaces (549) can be closed so that cover (540) can hold fluids such as formalin.

After the combination of cassette tray (510) and cover (540) is inserted into jar (160), jar (160) may be transported to the pathology laboratory as shown in FIG. 7 and described above. The collected tissue samples may then be processed in accordance with the workflow (300) shown in FIG. 7. However, since cassette assembly (500) can be used in lieu of tissue processing cassette (200), it should be understood that certain steps may be omitted such as straining the collected samples as represented by box (350) and placing the collected samples into a tissue processing cassette (200) as represented by box (370). In addition, it should be understood that at any one or more of the steps depicted in FIG. 7, an operator may interact with the label on cassette tray (510) to confirm chain of custody of the collected tissue samples. By way of example only, this may include scanning computer readable mediums associated with the label, confirming information on the label with information on jar (160) or other components, or confirming information on the label with patient files.

### IV. Exemplary Manifold Cover

In some instances it may be desirable to combine certain elements of the analysis process of the collected tissue samples once the desired number of samples have been collected within cassette tray (510). As indicated above, manipulation of tissue samples generally risks degrading the quality of the tissue samples each time the tissue samples are manipulated due to the fragility of the tissue. Transferring tissue samples during the analysis process may just as equally result in at least some manipulation of the tissue samples as in the transferring process between tissue sample tray (100) and tissue processing cassette (200) described above. Thus, transferring tissue samples between various elements may be undesirable in certain circumstances because this can lead to degradation of tissue sample quality. It is therefore desirable to reduce the number of containers used to deposit and analyze the tissue samples through. Providing fewer instances of transferring the tissue samples once collected within slidable member (620) described above may be beneficial to maintain the integrity of the samples beyond the collection process.

A. Cover with Screened Surface

FIG. 26 shows an exemplary slidable cover (700) that is generally configured to slidably engage slidable member (620) described above. It should be understood that slidable cover (700) of the present example may be readily incorporated into slidable member (620) described above. Slidable cover (700) comprises a receiving end (702), a closed end (704), and a pair of flanges (706) extending between receiving end (702) and closed end (704) to define a track (708) extending along the longitudinal length of slidable member (700). Receiving end (702) is sized and shaped in accordance with the size and shape of base (621) of slidable member (620). Accordingly, flanges (706) and track (708) are sized and shaped to correspond with the length and width of base (621) of slidable member (620). In other words, flanges (706) and track (708) are configured to slidably receive base (621) of slidable member (620) at receiving end (702). Slidable cover (700) further comprises a fastening mechanism (710) at receiving end (702). Fastening mechanism (710) is configured to securely fasten slidable cover (700) to slidable member (620). By way of example only, fastening mechanism (710) is a flexible clip (712) that is configured to engage base (621) when slidable member (620) is slidably inserted into track (708).

Flexible clip (712) is operable to deflect into and out of track (708) from an application of a predetermined force onto flexible clip (712). As it will be apparent to those of ordinary skill in the art, the direction in which flexible clip (712) deflects toward is dependent on the direction that the predetermined force is applied from. Flexible clip (712) is operable to deflect relative to track (708) due to a pair of slits (714) positioned adjacent to flexible clip (712) and which define flexible clip (712). Although not shown, it should be understood that fastening mechanism (712) may take other suitable forms than fastening clip (712) as will be apparent to those of ordinary skill in the art in view of the teachings herein.

As further seen in FIG. 26, in the present example flexible clip (712) includes a protruding abutment (716) extending into track (708). Protruding abutment (716) is configured to flexibly deflect with flexible clip (712) and is further configured to engage an edge portion of base (621) once slidable member (620) is fully inserted into track (708). Slidable cover (700) further comprises a screen surface (718) opposite of track (708). Screen surface (718) comprises a plurality of slots (719) extending between the pair of flanges (706) and positioned between the receiving end (702) and the closed end (704). Plurality of slots (719) of screen surface (718) are configured to provide fluid communication between an exterior of slidable cover (700) adjacent to screen surface (718) and an interior of slidable cover (700) adjacent to track (708). In this instance, with slidable cover (700) assembled onto a second feature, such as slidable member (620), a fluid may be transferred through slidable cover (700) via plurality of slots (719) to thereby expose the internal contents of the enclosed feature to the fluid. In other words, as will be described in greater detail below, a fluid such as formalin may be inserted into slidable member (620) through slots (719) despite slidable cover (700) being assembled thereon such that the tissue samples contained within slidable member (620) may be effectively exposed to the formalin while slidable cover (700) retains the tissue samples therein. As will further be described below, the assembly of slidable cover (700) and slidable member (620) may be positioned within a jar (160) containing a fluid, such as formalin, which thereby is able to enter into slidable member (620) through slidable cover (700) to thereby expose the tissue samples contained therein to the formalin.

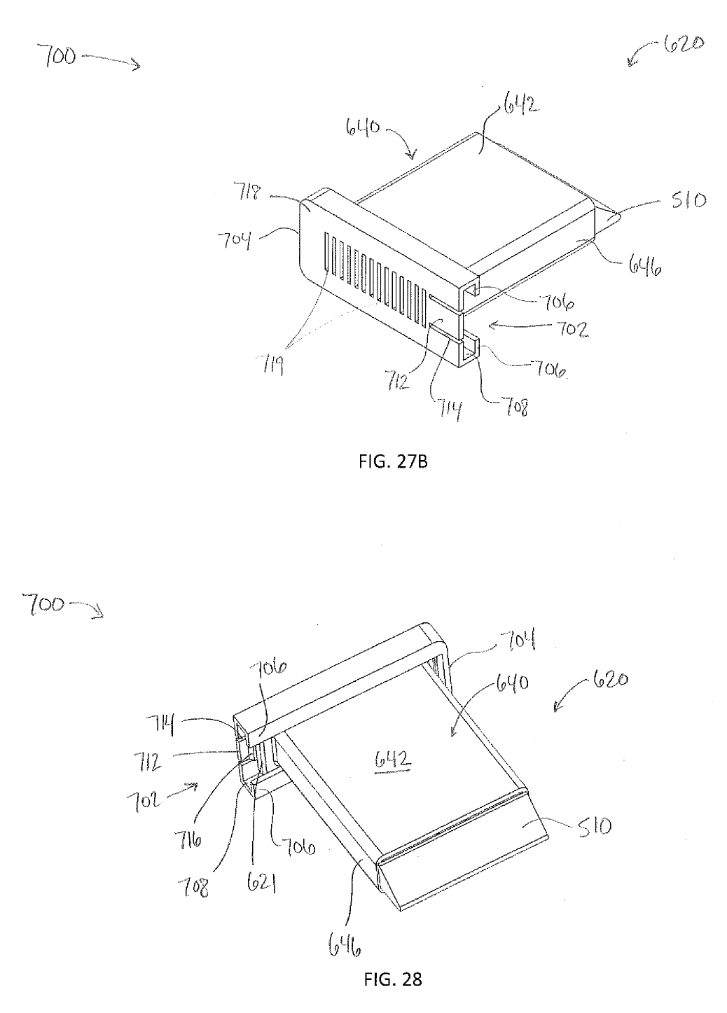

FIGS. 27A-27B show an exemplary use of slidable cover (700) to cover slidable member (620) of tissue sample holder assembly (600) once tissue samples have been collected within manifold (640). In particular, FIG. 27A shows slidable member (620) after having been recently disengaged from coupler (610) of tissue sample holder assembly (600) through the collection indexing process described above. Manifold (640) of slidable member (620) still contains cassette tray (510) within inner chamber (648) and it should be understood that cassette tray (510) contains at least one tissue sample deposited therein. In this instance, slidable cover (700) is advanced toward slidable member (620) to thereby securely fasten slidable cover (700) to slidable member (620). In particular, track (708) is aligned with base (621) at receiving end (702) to thereby slide flanges (706) over base (621) and thereby engage slidable cover (700) to slidable member (620). As base (621) is slidably inserted into track (708), protruding abutment (716) of fastening clip (712) initially engages base (621) and is forced outwardly relative to track (708). In this instance, as base (621) is further advanced between flanges (706), fastening clip (712) remains outwardly flexed until slidable member (620) is fully inserted into slidable cover (700), as seen in FIG. 27B.

With slidable member (620) fully inserted through track (708), a lateral force is no longer applied against protruding abutment (716) such that flexible clip (712) is operable to flex inwardly toward track (708) back to an original orientation, as best seen in FIG. 28. In this instance, protruding abutment (716) is positioned adjacent to base (621) of slidable member (620) such that protruding abutment (716) abuts base (621) and securely holds slidable member (620) within track (708). With slidable member (620) securely fastened to slidable cover (700), the tissue samples deposited within cassette tray (510) are prevented from exiting their respective sample chambers (523) due to the presence of slidable cover (700) positioned over access openings (622, 623, 624, 625) of manifold (640). However, due to the inclusion of plurality of slots (719) extending between screen surface (718) and track (708), fluid communication is permitted between an exterior of slidable cover (700) and inner chamber (648) of manifold (640) through plurality of slots (719) and access openings (622, 623, 624, 625).

The combination of slidable cover (700) and slidable member (620) be then inserted into jar (160) described above. As described above, jar (160) may be filled with a fluid such as formalin to preserve the collected tissue samples during transport and/or storage. Despite cassette tray (510) being positioned within inner chamber (648) of manifold (640), the tissue samples contained within cassette tray (510) will still be exposed to the formalin through the access provided into inner chamber (648) through the fluid communication between plurality of slots (719) and access openings (622, 623, 624, 625) as described above. Thus, slidable cover (700) provides a cover to prevent the tissue samples from exiting cassette tray (510) through access openings (622, 623, 624, 625) when slidable member (620) is positioned within jar (160) while simultaneously allowing fluid access to the tissue samples through screen surface (718). Although cassette tray (510) is described herein as being used with the same jar (160) described above, it should be understood that other alternative jars or containers may be used for transport and/or storage of cassette tray (510). For instance, in some examples jar (160) may be replaced with a container of a variety of shapes and sizes.

Alternatively, in other instances the tissue samples contained in cassette tray (510), which is contained within manifold (640) of slidable member (620), may be exposed to the fluid or formalin by directly injecting the fluid into cassette tray (510) through slidable cover (700). In this example, slidable cover (700) is slidably engaged to slidable member (620) as described above. However, rather than inserting the combination of slidable cover (700) and slidable member (620) into jar (160), the fluid or formalin may be inserted into cassette tray (510) by introducing the fluid at screen surface (718) such that the fluid passes through plurality of slots (719) and access openings (622, 623, 624, 625), respectively. In this instance, the fluid enters inner chamber (648) where cassette tray (510) is contained and the tissue samples deposited within sample chambers (523) of cassette tray (510) are exposed to the fluid to preserve the collected tissue samples during transport and/or storage. At this point the combination of slidable cover (700) and slidable member (620) is inserted into jar (160) such that jar (160) may be transported to the pathology laboratory as shown in FIG. 7 and described above.

The collected tissue samples may then be processed in accordance with the workflow (300) shown in FIG. 7. However, since cassette assembly (500) can be used in lieu of tissue processing cassette (200), it should be understood that certain steps may be omitted such as straining the collected samples as represented by box (350) and placing the collected samples into a tissue processing cassette (200) as represented by box (370). In addition, it should be understood that at any one or more of the steps depicted in FIG. 7, an operator may interact with the label on cassette tray (510) to confirm chain of custody of the collected tissue samples. By way of example only, this may include scanning computer readable mediums associated with the label, confirming information on the label with information on jar (160) or other components, or confirming information on the label with patient files.

B. Screen with Sealed Surface

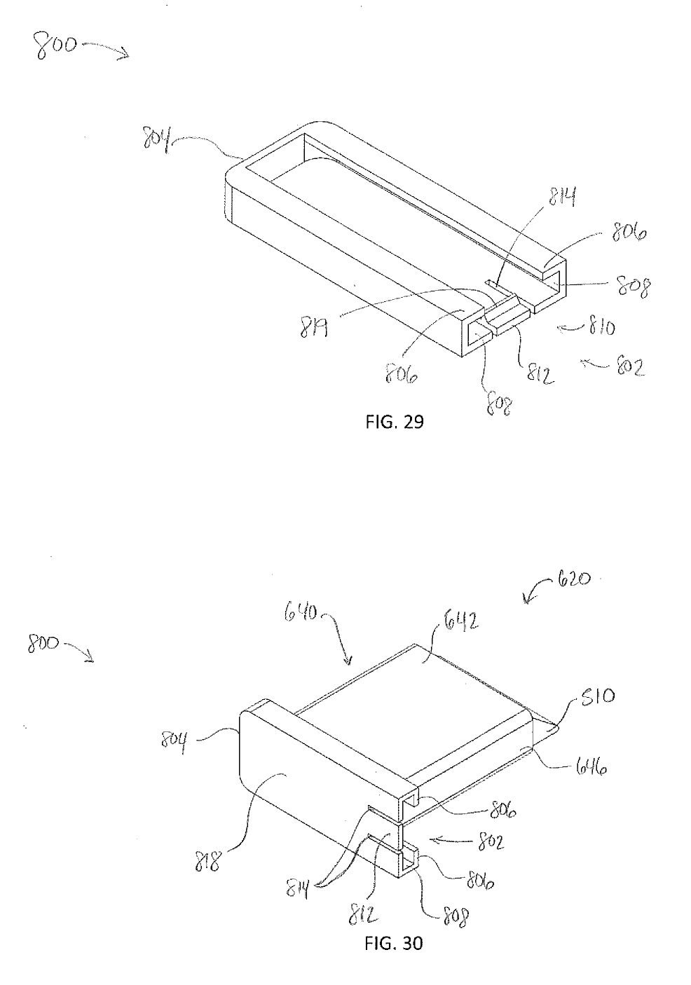

In other examples, an exemplary alternative slidable cover (800) may be fastened to slidable cover (620). It should be understood that slidable cover (800) of this example may be configured and operable just like slidable cover (700) described above, except for the differences explicitly noted herein. For instance, as shown in FIG. 29, slidable cover (800) comprises a receiving end (802), a closed end (804) and a pair of flanges (806) extending between receiving end (802) and closed end (804) to define a track (808) extending along the longitudinal length of slidable member (800). Except as otherwise described below, receiving end (802), closed end (804), pair of flanges (806) and track (808) may be configured and operable just like receiving end (702), closed end (704), pair of flanges (706) and track (708), respectively, described above.

Slidable cover (800) of the present example is configured to slidably engage base (621) of slidable member (620) in a substantially similar manner as slidable cover (700). For instance, similar to slidable cover (700), slidable cover (800) comprises a fastening mechanism (810) at receiving end (802) that is configured to securely fasten slidable cover (800) to slidable member (620). By way of example only, fastening mechanism (810) is a flexible clip (812) that is configured to engage base (621) when slidable member (620) is slidably inserted into track (808) of slidable cover (800).

However, unlike slidable cover (700), slidable cover (800) does not include a screen surface opposite of track (808) comprising a plurality of slots. Rather, slidable cover (800) includes a sealed surface (818) such that when slidable member (620) is securely fastened to slidable cover (800) any fluid communication between the tissue samples deposited within cassette tray (510) and the exterior of manifold (640) is effectively terminated. As seen in FIG. 30, sealed surface (818) provides a continuous plane extending between receiving end (802) and closed end (804) without any slots or apertures located therein such that sealed surface (818) is configured to create a barrier between track (808) and sealed surface (818). As will be discussed in greater detail below, sealed surface (818) is operable to form a sealed container with slidable member (620) by covering access openings (622, 623, 624, 625) such that any tissue samples deposited within cassette tray (510) when cassette tray (510) is contained within manifold (640) and slidable member (620) are isolated from any fluids, atmospheric air, or other contents exterior to inner chamber (648) of manifold (640).

In use, prior to attaching slidable cover (800) to base (621) of slidable cover (620), the tissue samples contained in cassette tray (510), which is contained within manifold (640) of slidable member (620), are exposed to a fluid or formalin by directly injecting the fluid into cassette tray (510) through access openings (622, 623, 624,625) of manifold (640). In this example, fluid communication to cassette tray (510) is still available as slidable cover (800) has yet to be attached to slidable member (620). However, once the fluid or formalin is inserted into cassette tray (510) through access openings (622, 623, 624, 625), respectively, base (621) of slidable member (620) is slidably inserted into track (808) of slidable cover (800), as similarly described above with respect to slidable cover (700). In this instance, the fluid is contained within inner chamber (648) where cassette tray (510) is contained and the tissue samples deposited within sample chambers (523) of cassette tray (510) are exposed to the fluid to preserve the collected tissue samples during transport and/or storage. At this point the combination of slidable cover (800) and slidable member (620) is inserted into jar (160) such that jar (160) may be transported to the pathology laboratory as shown in FIG. 7 and described above. Alternatively, it should be understood that use of jar (160) in this instance is merely optional as slidable cover (800) effectively encloses slidable member (620) such that the formalin contained therein is adequately sealed in. Thus, the use of jar (160) is not necessary to contain the formalin within cassette tray (510). Including any fluid or formalin in jar (160) is also not necessary in this example as inner chamber (648) of manifold (640) already contains such fluid and because slidable cover (800) effectively prevents any exterior fluid from communicating with inner chamber (648) once engaged to slidable member (620). The collected tissue samples may then be processed in accordance with the workflow (300) shown in FIG. 7.

### V. Exemplary Remote Tissue Collection and Processing System

In some instances, it may be beneficial to immediately observe the tissue samples extracted with a biopsy instrument during a medical procedure as soon as the sample is deposited in the tissue sample holder assembly. However, due to the relatively inaccessible position of the biopsy instrument during a medical procedure, an operator may be unable to visually inspect the tissue samples deposited in the tissue sample holder assembly until after the procedure is completed, the biopsy instrument is removed from the biopsy site, and the tissue sample holder assembly is detached from the instrument. In part, the inability to visually inspect the tissue samples extracted by the biopsy instrument is due to the position of the tissue sample holder assembly relative to the biopsy instrument. As described above, tissue sample holder assembly (40, 600) is attached to a proximal end of biopsy device (10) such that tissue sample holder assembly (40, 600) is positioned with biopsy device (10) at the biopsy site. Accordingly, providing a collection mechanism that allows for tissue sample holder assembly (40, 600) to be remotely positioned at a location adjacent to an operator while biopsy device (10) remains positioned adjacent to the biopsy site may be desirable to allow for the immediate inspection of tissue samples extracted by biopsy device (10).

The following description provides various examples of a remote tissue collection mechanism configured to transport the tissue samples extracted by a biopsy instrument, such as biopsy device (10) described above, from a biopsy site to a tissue sample holder assembly, such as tissue sample holder assembly (40, 600) described above, that is located at a remote position relative to the biopsy instrument and biopsy site. Ultimately, remotely locating tissue sample holder assembly (40, 600) to a location adjacent to an operator may be beneficial to provide real-time inspection of any tissue samples extracted by biopsy device (10) throughout the medical procedure.

It should be understood that the remote tissue collection mechanism described below may be readily incorporated into any of the various biopsy devices (10) and tissue sample holder assemblies (40, 600) described above and in any of the various surgical procedures described in the various references herein. Other suitable ways in which the below-described remote tissue collection mechanism may be used will be apparent to those of ordinary skill in the art in view of the teachings herein.

A. Remote Tissue Collection with Indexing Tissue Sample Holder Assembly

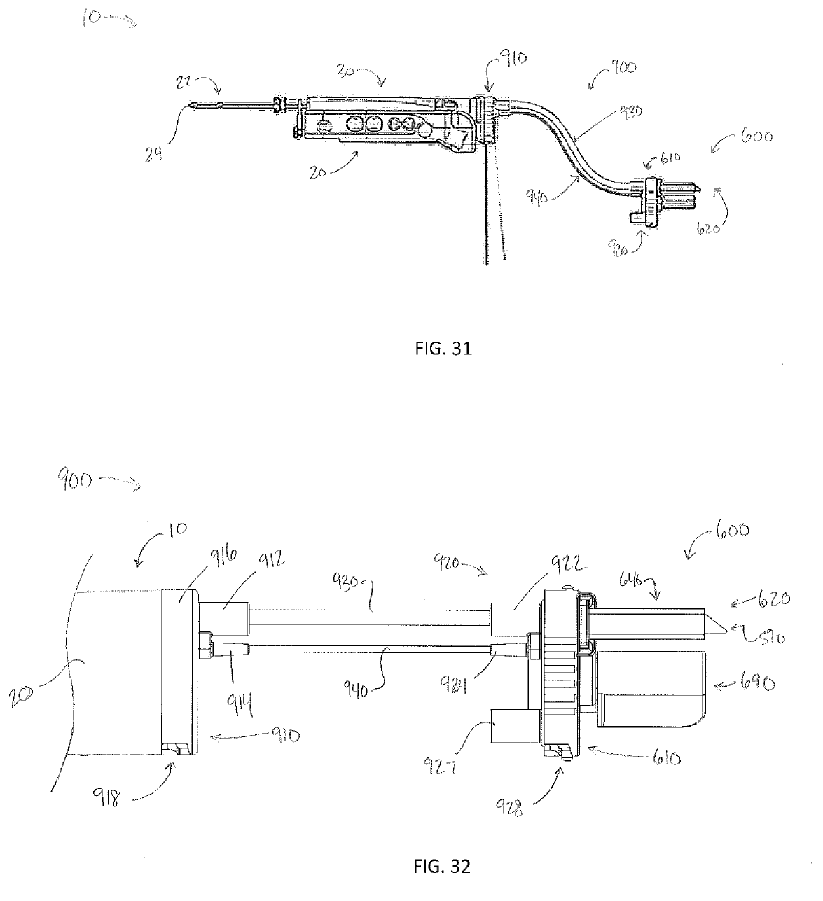

FIG. 31 shows an exemplary remote tissue collection system (900) coupled to biopsy device (10) and tissue sample holder assembly (600). It should be understood that remote tissue collection system (900) of the present example may be readily incorporated into biopsy device (10) and tissue sample holder assembly (600) described above. It should also be understood that, in many respects, biopsy device (10) and tissue sample holder assembly (600) function substantially similar as described above except as otherwise described below. In other words, biopsy device (10) and tissue sample holder assembly (600) are configured and operable as described above except for the differences described herein.

Remote tissue collection system (900) comprises a device coupler (910), a tissue holder coupler (920), a tissue transport conduit (930) and a fluid transport conduit (940). Remote tissue collection system (900) is generally configured to provide fluid communication between biopsy device (10) and tissue sample holder assembly (600) through conduits (930, 940) with biopsy device (10) and tissue sample holder assembly (600) detached and located at varying locations. In other words, remote tissue collection system (900) provides the ability for biopsy device (10) to be positioned adjacent to a biopsy site where tissue samples are extracted from a patient with the use of needle (22) while tissue sample holder assembly (600) is positioned at another location adjacent to an operator while still being able to receive the tissue samples extracted by biopsy device (10). Conduits (930, 940) are coupled to biopsy device (10) and tissue sample holder assembly (600) with device coupler (910) and tissue holder coupler (920), respectively, to provide fluid communication between biopsy device (10) and tissue sample holder assembly (600) such that tissue samples may be transported from device (10) to tissue sample holder assembly (600). It should be understood conduits (930, 940) may comprise various suitable lengths, profiles, and/or configurations as will be apparent to those of ordinary skill in the art in view of the teachings herein.

As best seen in FIG. 32, device coupler (910) is configured to removably couple to biopsy device (10) and includes a tissue port (912) and a fluid port (914) extending therefrom proximally relative to biopsy device (10). Device coupler (910) comprises a circular-shaped body (916) that is shaped and sized to attach to a proximal end of probe assembly (20). In particular, circular-shaped body (916) of device coupler (910) is configured to removably couple to probe assembly (20) at the proximal end where tissue sample holder assembly (600) would have otherwise been directly coupled to biopsy device (10). Tissue port (912) is sized and shaped to receive a distal end of tissue transport conduit (930). Tissue port (912) is configured to transport tissue samples extracted by the cutter of biopsy device (10) through device coupler (910) and into tissue transport conduit (930). Similarly, fluid port (914) is sized and shaped to receive a distal end of fluid transport conduit (940). Fluid port (914) is configured to receive fluid delivered from tissue sample holder assembly (600) through tissue holder coupler (920) and fluid transport conduit (940).

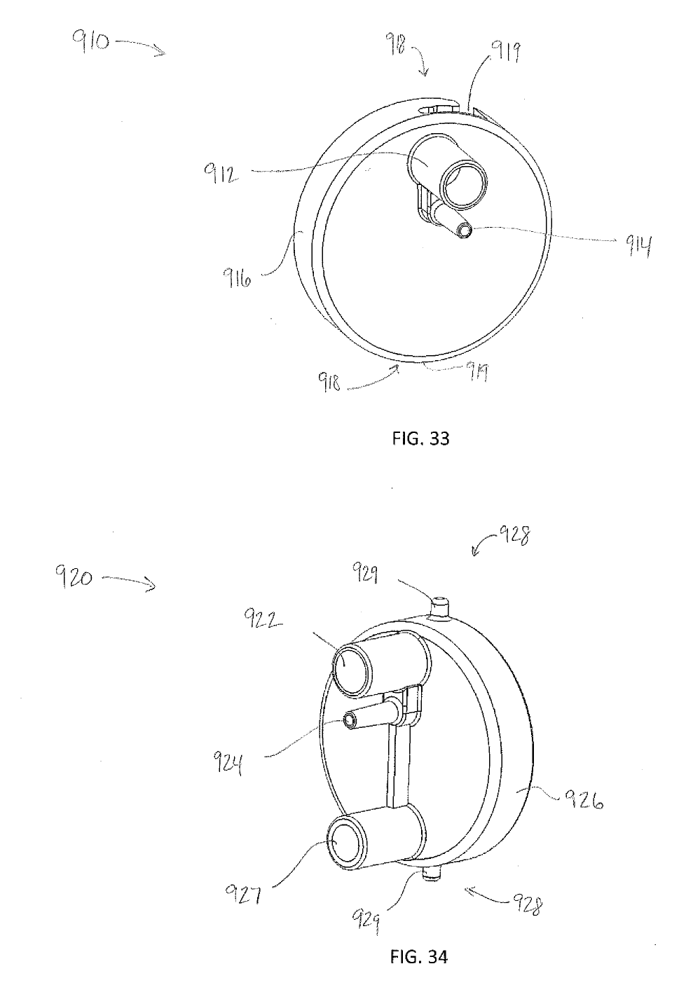

Device coupler (910) further includes a fastening mechanism (918) configured and operable to securely attach device coupler (910) to biopsy device (10). In the present example, fastening mechanism (918) is in the form of a pair of bayonet connectors (919) that are sized and configured to receive a pair of bayonet pins (not shown) of probe assembly (20) to selectively couple device coupler (910) to probe assembly (20), as best seen in FIG. 33. Thus, bayonet connectors (919) and the bayonet pins of probe assembly (20) form a standard bayonet coupling assembly to selectively secure device coupler (910) to probe assembly (20). In this configuration, circular-shaped body (916) is generally rotatable relative to probe assembly (20) to lock and unlock device coupler (910) relative to probe assembly (20). Although not shown, it should be understood that to assist an operator with rotation of circular-shaped body (916), device coupler (910) may include grips to enhance the grip of circular-shaped body (916) during locking and unlocking. Although the present example uses a bayonet coupling to secure device coupler (910) to probe assembly (20), it should be understood that in other examples various alternative coupling features can be used as will be apparent to those of ordinary skill in the art in view of the teachings herein.

Tissue holder coupler (920) is configured to removably couple to tissue sample holder assembly (600) and includes a tissue port (922) and a fluid port (924) extending therefrom distally relative to tissue sample holder assembly (600). Tissue holder coupler (920) comprises a circular-shaped body (926) that is shaped and sized to attach to coupler (610) of tissue sample holder assembly (600). In particular, circular-shaped body (926) of tissue holder coupler (920) is configured to removably couple to coupler (610) by entering into ring-shaped body (612) and engaging sealing edge (614) to thereby form a sealed connection with tissue sample holder assembly (600). Tissue port (922) is sized and shaped to receive a proximal end of tissue transport conduit (930). Tissue port (922) is configured to receive tissue samples extracted by the cutter of biopsy device (10) and transported through device coupler (910) and tissue transport conduit (930). Similarly, fluid port (924) is sized and shaped to receive a proximal end of fluid transport conduit (940). Fluid port (924) is configured to transport fluid contained within cassette tray (510) from the tissue samples deposited in tissue sample holder assembly (600) through tissue holder coupler (920) and fluid transport conduit (940).

Tissue holder coupler (920) further includes a fastening mechanism (928) configured and operable to securely attach tissue holder coupler (920) to tissue sample holder assembly (600). In the present example, fastening mechanism (928) is in the form of a pair of bayonet pins (929) that are sized and configured to be received the pair of bayonet connectors (616) of coupler (610) to selectively couple tissue holder coupler (920) to tissue sample holder assembly (600), as best seen in FIG. 34. Thus, bayonet pins (929) and bayonet connectors (616) of coupler (610) form a standard bayonet coupling assembly to selectively secure tissue holder coupler (920) to tissue sample holder assembly (600). In this configuration, circular-shaped body (926) is generally rotatable relative to coupler (610) to lock and unlock tissue holder coupler (920) relative to coupler (610). Although not shown, it should be understood that to assist an operator with rotation of circular-shaped body (926), tissue holder coupler (920) may include grips to enhance the grip of circular-shaped body (926) during locking and unlocking. Although the present example uses a bayonet coupling to secure tissue holder coupler (920) to tissue sample holder assembly (600), it should be understood that in other examples various alternative coupling features can be used as will be apparent to those of ordinary skill in the art in view of the teachings herein.

Tissue holder coupler (920) further includes a handle feature (927) extending outwardly from tissue holder coupler (920). Handle feature (927) is configured to provide an operator with an additional surface or portion of tissue holder coupler (920) to grasp when attaching or detaching tissue holder coupler (920) to device coupler (910) of tissue sample holder (600). Handle feature (927) is operable to be manipulated to further tighten tissue holder coupler (920) to device coupler (910) after tissue holder coupler (920) is initially fastened to tissue sample holder assembly (600). In the present example, handle feature (927) protrudes from tissue holder coupler (920) at a predetermined length relatively similar to tissue port (922). Handle feature (927) similarly comprises a cylindrical shape. Although not shown, it should be understood that handle feature (927) may comprise other suitable shapes, sizes, and configurations as will be apparent to those of ordinary skill in the art.

In use, device coupler (910) is securely fastened to probe assembly (20) of biopsy device (10) and tissue holder coupler (920) is securely fastened to coupler (610) of tissue sample holder assembly (600). With couplers (910, 920) attached to biopsy device (10) and tissue sample holder assembly (600), respectively, tissue transport conduit (930) is connected to ports (912, 922) while fluid transport conduit (940) is connected to ports (914, 924). It should be understood that that order in which couplers (910, 920) and conduits (930, 940) are assembled or connected relative to each other is irrelevant such that conduits (930, 940) can be connected to ports (912, 922, 914, 924) prior to couplers (910, 920) being coupled to biopsy device (10) and tissue sample holder assembly (600), respectively. Similarly, the order in which couplers (910, 920) are attached to biopsy device (10) and tissue sample holder assembly (600), respectively, is also immaterial such that either coupler (910, 920) may be attached before the other. Further, the order in which conduits (930, 940) are coupled to couplers (910, 920) is insignificant.

In this instance, with couplers (910, 920) and conduits (930, 940) securely coupled to each other, biopsy device (10), and tissue sample holder assembly (600), fluid communication is effectively created between biopsy device (10) and tissue sample holder assembly (600). In this instance, an operator may selectively position biopsy device (10) at a location adjacent to the biopsy site suitable for performing the medical procedure with the cutter of biopsy device (10). An operator may selectively position tissue sample holder assembly (600) at an alternate location relative to biopsy device (10) such that the tissue sample holder assembly (600) is positioned adjacent to an operator suitable for viewing the tissue samples extracted by the cutter of biopsy device (10) and subsequently deposited in tissue sample holder assembly (600). In particular, actuation of the cutter of biopsy device (10) will result in severing a tissue sample from the patient and temporarily depositing the tissue sample in needle (22). Through a vacuum flow generated by probe assembly (20), the tissue sample is pulled proximally through needle (22), probe assembly (20), and toward device coupler (910) until reaching tissue port (912). In this instance, the tissue sample is transported through tissue transport conduit (930) until reaching tissue transport port (922) of tissue holder coupler (920).

The tissue sample is subsequently transferred through coupler (610) and into access port (611). Depending on the particular access opening (622, 623, 624, 625) of slidable member (620) that is indexed into alignment with access port (611) of coupler (610), a particular sample chamber (523) of cassette tray (510) will receive the tissue sample therein. Simultaneous with the transport of the tissue sample through tissue transport conduit (930) in a proximal direction toward tissue sample holder assembly (600), made possible by the vacuum generated through probe assembly (20), any excess fluids released by the tissue samples deposited in manifold (640) are transported from inner chamber (648) and into the particular vent opening (626, 627, 628, 629) that is aligned with vent port (613) of coupler (610). In this instance, the fluid is pulled through tissue holder coupler (920), fluid port (924), and fluid transport conduit (940) until reaching fluid port (914) of device coupler (910) where the fluid will be redirected through probe assembly (20) as described above.

It should be understood that conduits (930, 940) are formed of a flexible material such that the profile and configuration of conduits (930, 940) may be selectively adjusted before, during, and after a procedure. It should be further understood that since conduits (930, 940) are securely coupled to couplers (910, 920) through the engagement with ports (912, 922, 914, 924), the positions of biopsy device (10) and/or tissue sample holder assembly (600), respectively, may be selectively relocated within a procedure room as necessary during a procedure such that remote tissue collection system (900) does not need to be disassembled and subsequently reassembled. Due to the selective maneuverability of biopsy device (10), an operator may reposition biopsy device (10) at various biopsy sites relative to a patient during the medical procedure without requiring reinstallation of remote tissue collection system (900). Similarly, an operator may reposition tissue sample holder assembly (600) at various locations in a procedure room relative to biopsy device (10) to perform various functions. By way of example only, tissue sample holder assembly (600) may be relocated adjacent to tissue scanning devices, such as x-ray, optical coherence topography, mass spectrometry, electrical impedance, and/or etc., to aid an operator in inspecting the tissue samples deposited therein. In this instance, prior to inserting cassette tray (510), and/or the combination of slidable member (620) and cassette tray (510), into formalin jar (160), an operator may generate images of the tissue samples contained therein by repositioning tissue sample holder assembly (600) next to an imaging device.

B. Remote Tissue Collection with Rotatable Tissue Sample Holder Assembly

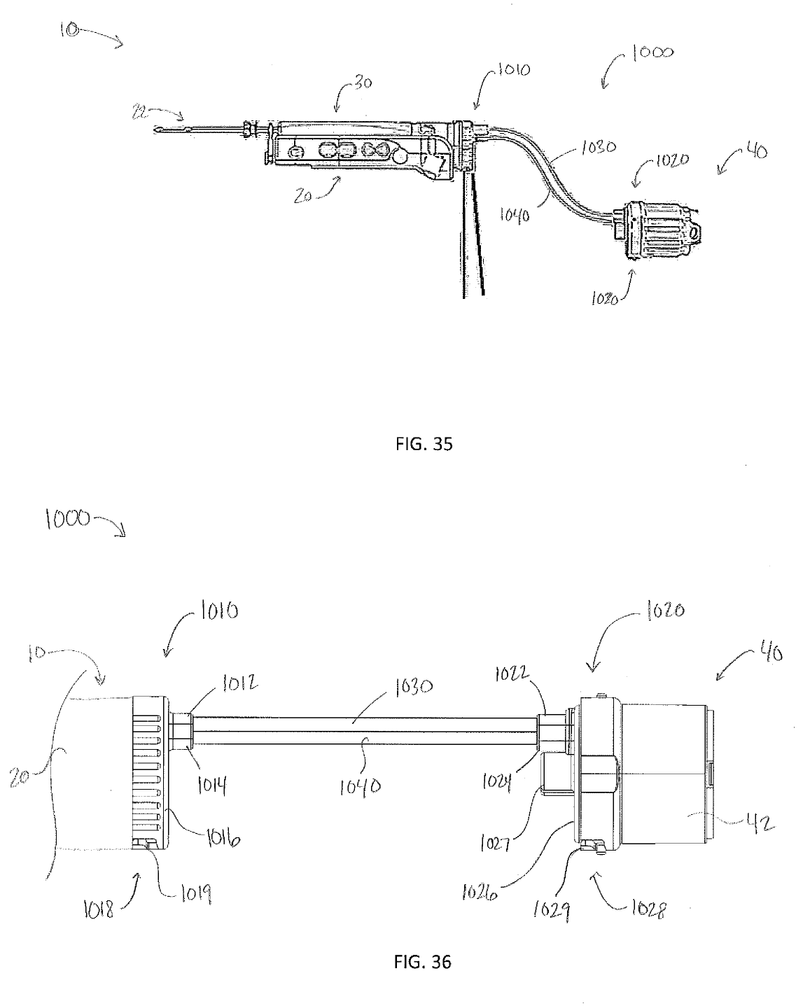

In other examples, an exemplary alternative remote tissue collection system (1000) may be coupled to biopsy device (10) and tissue sample holder assembly (40). It should be understood that remote tissue collection system (1000) of this example may be configured and operable just like remote tissue collection system (900) described above, except for the differences explicitly noted herein. For instance, as shown in FIG. 35, remote tissue collection system (1000) comprises a device coupler (1010), a tissue holder coupler (1020), a tissue transport conduit (1030), and a fluid transport conduit (1040). Except as otherwise described below, device coupler (1010), a tissue holder coupler (1020), a tissue transport conduit (1030), and a fluid transport conduit (1040) may be configured and operable just like device coupler (910), tissue holder coupler (920), tissue transport conduit (930), and fluid transport conduit (940), respectively, described above. It should be also understood that remote tissue collection system (1000) of the present example may be readily incorporated into biopsy device (10) and tissue sample holder assembly (40) described above. In many respects, biopsy device (10) and tissue sample holder assembly (40) function substantially similar as described above except as otherwise described below. In other words, biopsy device (10) and tissue sample holder assembly (40) are configured and operable as described above except for the differences described herein.

Remote tissue collection system (1000) comprises a device coupler (1010), a tissue holder coupler (1020), a tissue transport conduit (1030) and a fluid transport conduit (1040). Remote tissue collection system (1000) is generally configured to provide fluid communication between biopsy device (10) and tissue sample holder assembly (40) through conduits (1030, 1040) with biopsy device (10) and tissue sample holder assembly (40) detached and located at varying locations. In other words, remote tissue collection system (1000) provides the ability for biopsy device (10) to be positioned adjacent to a biopsy site where tissue samples are extracted from a patient with the use of needle (22) while tissue sample holder assembly (40) is positioned at another location adjacent to an operator while still being able to receive the tissue samples extracted by biopsy device (10). Conduits (1030, 1040) are coupled to biopsy device (10) and tissue sample holder assembly (40) with device coupler (1010) and tissue holder coupler (1020), respectively, to provide fluid communication between biopsy device (10) and tissue sample holder assembly (40) such that tissue samples may be transported from device (10) to tissue sample holder assembly (40). It should be understood conduits (1030, 1040) may comprise various suitable lengths, profiles, and/or configurations as will be apparent to those of ordinary skill in the art in view of the teachings herein.

As seen in FIG. 36, device coupler (1010) is configured to removably couple to biopsy device (10) and includes a tissue port (1012) and a fluid port (1014) extending therefrom proximally relative to biopsy device (10). Device coupler (1010) comprises a circular-shaped body (1016) that is shaped and sized to attach to a proximal end of probe assembly (20). In particular, circular-shaped body (1016) of device coupler (1010) is configured to removably couple to probe assembly (20) at the proximal end where tissue sample holder assembly (40) would have otherwise been directly coupled to biopsy device (10). Tissue port (1012) is sized and shaped to receive a distal end of tissue transport conduit (1030). Tissue port (1012) is configured to transport tissue samples extracted by the cutter of biopsy device (10) through device coupler (1010) and into tissue transport conduit (1030). Similarly, fluid port (1014) is sized and shaped to receive a distal end of fluid transport conduit (1040). Fluid port (1014) is configured to receive fluid delivered from tissue sample holder assembly (40) through tissue holder coupler (1020) and fluid transport conduit (1040).

Device coupler (1010) further includes a fastening mechanism (1018) configured and operable to securely attach device coupler (1010) to biopsy device (10). In the present example, fastening mechanism (1018) is in the form of a pair of bayonet connectors (1019) that are sized and configured to receive a pair of bayonet pins (not shown) of probe assembly (20) to selectively couple device coupler (1010) to probe assembly (20). Thus, bayonet connectors (1019) and the bayonet pins of probe assembly (20) form a standard bayonet coupling assembly to selectively secure device coupler (1010) to probe assembly (20). In this configuration, circular-shaped body (1016) is generally rotatable relative to probe assembly (20) to lock and unlock device coupler (1010) relative to probe assembly (20). Although not shown, it should be understood that to assist an operator with rotation of circular-shaped body (1016), device coupler (1010) may include grips to enhance the grip of circular-shaped body (1016) during locking and unlocking. Although the present example uses a bayonet coupling to secure device coupler (1010) to probe assembly (20), it should be understood that in other examples various alternative coupling features can be used as will be apparent to those of ordinary skill in the art in view of the teachings herein.

Tissue holder coupler (1020) is configured to removably couple to tissue sample holder assembly (40) and includes a tissue port (1022) and a fluid port (1024) extending therefrom distally relative to tissue sample holder assembly (40). Tissue holder coupler (1020) comprises a circular-shaped body (1026) that is shaped and sized to attach to tissue sample holder assembly (40). In particular, circular-shaped body (1026) of tissue holder coupler (1020) is configured to removably couple to tissue sample holder assembly (40) by entering cylindraceous outer cover (42) to thereby form a sealed connection with tissue sample holder assembly (40). Tissue port (1022) is sized and shaped to receive a proximal end of tissue transport conduit (1030). Tissue port (1022) is configured to receive tissue samples extracted by the cutter of biopsy device (10) and transported through device coupler (1010) and tissue transport conduit (1030). Similarly, fluid port (1024) is sized and shaped to receive a proximal end of fluid transport conduit (1040). Fluid port (1024) is configured to transport fluid contained within sample trays (100) from the tissue samples deposited in tissue sample holder assembly (40) through tissue holder coupler (1020) and fluid transport conduit (1040).

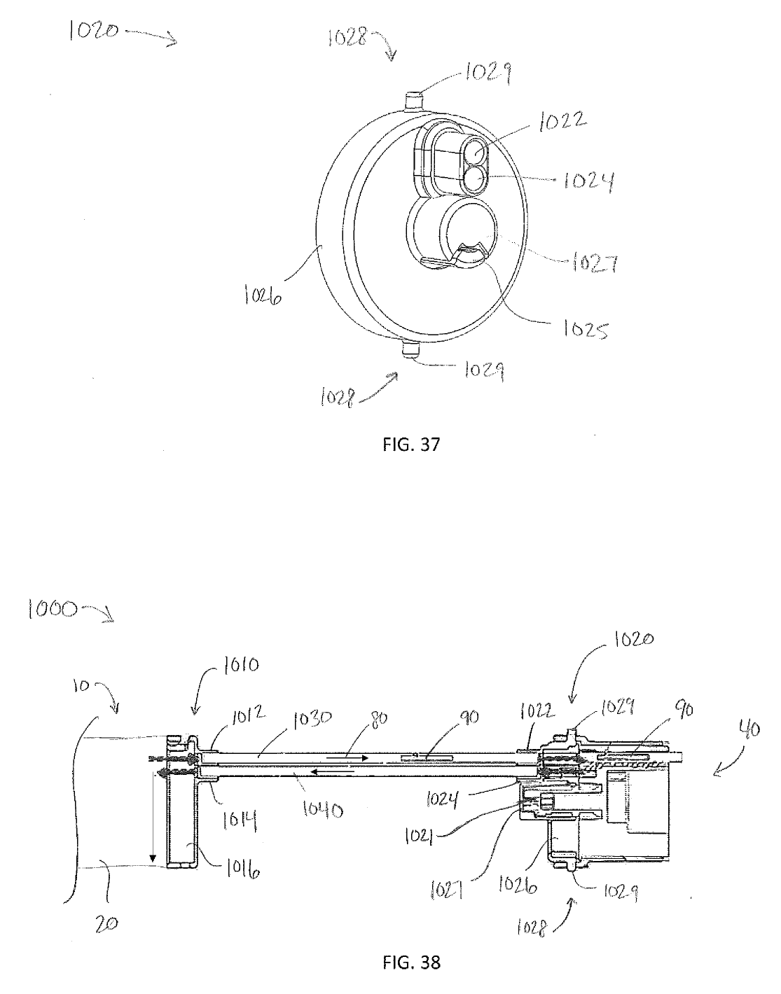

Tissue holder coupler (1020) further includes a fastening mechanism (1028) configured and operable to securely attach tissue holder coupler (1020) to tissue sample holder assembly (40). In the present example, fastening mechanism (1028) is in the form of a pair of bayonet pins (1029) that are sized and configured to be received the pair of bayonet connectors of cylindraceous outer cover (42) to selectively couple tissue holder coupler (1020) to tissue sample holder assembly (40), as best seen in FIG. 37. Thus, bayonet pins (1029) and bayonet connectors of cylindraceous outer cover (42) form a standard bayonet coupling assembly to selectively secure tissue holder coupler (1020) to tissue sample holder assembly (40). In this configuration, circular-shaped body (1026) is generally rotatable relative to cylindraceous outer cover (42) to lock and unlock tissue holder coupler (1020) relative to cylindraceous outer cover (42). Although not shown, it should be understood that to assist an operator with rotation of circular-shaped body (1026), tissue holder coupler (1020) may include grips to enhance the grip of circular-shaped body (1026) during locking and unlocking. Although the present example uses a bayonet coupling to secure tissue holder coupler (1020) to tissue sample holder assembly (40), it should be understood that in other examples various alternative coupling features can be used as will be apparent to those of ordinary skill in the art in view of the teachings herein.

Tissue holder coupler (1020) further includes a collar (1027) positioned adjacent to ports (1022, 1024). Collar (1027) protrudes outwardly from tissue sample coupler (1020) in a proximal direction similar to ports (1022, 1024) such that a cavity (1021) is formed within collar (1027), as best seen in FIG. 38. Cavity (1021) is sized and shaped to receive at least a portion of tissue sample holder (40) once tissue holder coupler (1020) is coupled to tissue sample holder (40). As best seen in FIG. 37, collar (1027) includes an indexing tab (1025) that is sized and shaped to associate with the portion of tissue sample holder (40) positioned within cavity (1021). In other words, indexing tab (1025) includes a protrusion (not shown) extending into cavity (1021) and that is configured to engage at least the portion of tissue sample holder (40) received within cavity (1021). As merely an illustrative example, the portion of tissue sample holder (40) positioned within cavity (1021) of collar (1027) is a gear including a plurality of teeth such that the protrusion of indexing tab (1025) is sized and shaped to mesh with the plurality of teeth.

Indexing tab (1025) is resiliently biased such that the protrusion extends inwardly into cavity (1021) such that indexing tab (1025) is configured to interlock with the portion of tissue sample holder (40) positioned within cavity (1021) to thereby inhibit rotation of tissue sample holder (40) relative to tissue holder coupler (1020). With tissue sample holder (40) coupled to tissue holder coupler (1020), indexing tab (1025) is engaged with the portion of tissue sample holder (40) contained in collar (1027) such that the orientation of tissue sample holder (40) relative to tissue holder coupler (1020) is fixed. Indexing tab (1025) is configured to permit tissue sample holder (40) to rotate relative to tissue holder coupler (1020) and to release tissue sample holder (40) from the fixed orientation in response to a predetermined rotatable force applied onto tissue sample holder (40). In this instance, applying a rotatable force at a distal end of tissue sample holder (40) temporarily disengages the portion of tissue sample holder (40) positioned within cavity (1021) from its engagement with the protrusion of indexing tab (1025). In particular, the protrusion of indexing tab (1025) is biased outwardly from within cavity (1021) when tissue sample holder (40) is rotated thereby permitting tissue sample holder (40) to rotate to a subsequent orientation until indexing tab (1025) resiliently extends the protrusion back into cavity (1021) and in engagement with the portion of tissue sample holder (40) contained therein.

In use, device coupler (1010) is securely fastened to probe assembly (20) of biopsy device (10) and tissue holder coupler (1020) is securely fastened to cylindraceous outer cover (42) of tissue sample holder assembly (40). With couplers (1010, 1020) attached to biopsy device (10) and tissue sample holder assembly (40), respectively, tissue transport conduit (1030) is connected to ports (1012, 1022) while fluid transport conduit (1040) is connected to ports (1014, 1024), as seen in FIG. 38. In this instance, with couplers (1010, 1020) and conduits (1030, 1040) securely coupled to each other, biopsy device (10), and tissue sample holder assembly (40), fluid communication is effectively created between biopsy device (10) and tissue sample holder assembly (40). In this instance, an operator may selectively position biopsy device (10) at a location adjacent to the biopsy site suitable for performing the medical procedure with the cutter of biopsy device (10). An operator may selectively position tissue sample holder assembly (40) at an alternate location relative to biopsy device (10) such that the tissue sample holder assembly (40) is positioned adjacent to an operator suitable for viewing the tissue samples extracted by the cutter of biopsy device (10) and subsequently deposited in tissue sample holder assembly (40). In particular, actuation of the cutter of biopsy device (10) will result in severing a tissue sample (90) from the patient and temporarily depositing tissue sample (90) in needle (22). Through a vacuum flow (80) generated by probe assembly (20), tissue sample (90) is pulled proximally through needle (22), probe assembly (20), and toward device coupler (1010) until reaching tissue port (1012). In this instance, tissue sample (90) is transported through tissue transport conduit (1030) until reaching tissue transport port (1022) of tissue holder coupler (1020).

Tissue sample (90) is subsequently transferred through a particular strip receiving chamber (46) of rotatable member (44) that is rotated in alignment with tissue port (1022) such that the particular tissue receiving chamber (120) that is received in the strip receiving chamber (46) will receive tissue sample (90) therein. Simultaneous with the transport of tissue sample (90) through tissue transport conduit (1030) in a proximal direction toward tissue sample holder assembly (40), made possible by the vacuum (80) generated through probe assembly (20), any excess fluids released by tissue samples (90) deposited in tissue sample trays (100) are transported from rotatable member (44) and into the particular pulled through tissue holder coupler (1020), fluid port (1024), and fluid transport conduit (1040) until reaching fluid port (1014) of device coupler (1010) where the fluid will be redirected through probe assembly (20) as described above.

Similar to conduits (930, 940) described above, conduits (1030, 1040) are formed of a flexible material such that the profile and configuration of conduits (1030, 1040) may be selectively adjusted before, during, and after a procedure. It should be further understood that the positions of biopsy device (10) and/or tissue sample holder assembly (40) may be selectively relocated within a procedure room as necessary during a procedure such that remote tissue collection system (1000) does not need to be disassembled and subsequently reassembled. Due to the selective maneuverability of biopsy device (10), an operator may reposition biopsy device (10) at various biopsy sites relative to a patient during the medical procedure without requiring reinstallation of remote tissue collection system (1000). Similarly, an operator may reposition tissue sample holder assembly (40) at various locations in a procedure room relative to biopsy device (10) to perform various functions. By way of example only, tissue sample holder assembly (40) may be relocated adjacent to tissue scanning devices, such as x-ray, optical coherence topography, mass spectrometry, electrical impedance, and/or etc., to aid an operator in inspecting tissue samples (90) deposited therein. In this instance, prior to extracting tissue samples (90) from tissue sample holder assembly (40), an operator may generate images of tissue samples (90) contained therein by repositioning tissue sample holder assembly (40) next to an imaging device.

### VI. Exemplary Alternative Integrated Tissue Collection and Processing System

In some instances, it may be desirable to combine certain elements of the tissue sample holder assembly (40) described above with the tissue analysis cassette (200) described above. For instance, manipulation of tissue samples generally risks degrading the quality of the tissue samples each time the tissue samples are manipulated due to the fragility of the tissue. Transferring tissue samples between elements like tissue sample tray (100) described above and tissue processing cassette (200) described above often result in at least some manipulation of the tissue samples being transferred. Thus, transferring tissue samples between various elements may be undesirable in certain circumstances because this can lead to degradation of tissue sample quality. It is therefore desirable to reduce the number of containers used to deposit tissue samples during the workflow (300) described above.

In addition to manipulation of tissue samples being generally undesirable, transferring tissue samples between different containers (e.g., tissue sample tray (100), tissue processing cassette (200)) can lead to mislabeling or tacking errors associated with tissue samples as the tissue samples progress through the workflow (300) described above. For instance, when tissue sample are transferred from tissue sample tray (100) to tissue processing cassette (200), incorrect patient information might be printed on tissue processing cassette (200). Another possibility is that an incorrect label may be placed on tissue processing cassette (200). Thus, transferring tissue samples between different containers also includes the risk of generating errors in tissue sample tracking. Accordingly, it is desirable to reduce the number of containers used in the workflow (300) described above to generally improve tissue sample integrity and reduce operator error.

Although various devices and methods are described below for reducing the number of containers used in the workflow (300) described above are described herein, it should be understood that various alternative configurations will be apparent to those of ordinary skill in the art in view of the teachings herein. For instance, some suitable alternative configurations may combine various features of one embodiment described below with various features of another alternative embodiment. Still other suitable alternative configurations may omit various features of one or more embodiments. Of course, other suitable configurations will be apparent to those of ordinary skill in the art in view of the teachings herein.

A. Alternative Slidable Tissue Sample Holder Assembly

FIGS. 39-40 show an exemplary alternative tissue sample holder assembly (1600) that may be used with biopsy device (10) in lieu of tissue sample holder assembly (40) described above. Tissue sample holder assembly (1600) comprises a coupler (1610), a slidable member (1620), and a track (680). Coupler (1610) comprises a generally ring-shaped body (1612) with a closed proximal end (1614), an elongated shaft (1616), and a plurality of grips (618). Closed proximal end (1614) is configured to engage at least a portion of probe assembly (20) to seal coupler (1610) to probe assembly (20). In addition, closed proximal end (1614) is configured to fixedly secure coupler (1610) to probe assembly (20) to thereby permit translation of slidable member (1620) relative to coupler (1610) within track (1680). As will be described in greater detail below, this translation permits tissue processing cassette (200) to be moved relative to probe assembly (20) so that at least one tissue sample can be collected within each channel (1698) of cassette insert (1690).

Elongated shaft (1616) defines an internal channel that is configured to be in fluid communication with needle (22) of biopsy device (10) such that shaft (1616) is operable to receive tissue samples extracted from a patient. Thus, elongated shaft (1616) is received by and connected to probe assembly (20) to form a connection with the cutter of biopsy device (10) to selectively pull tissue samples through probe assembly (20) and into tissue sample holder assembly (1600). Although elongated shaft (1616) is described as being an integral component with coupler (1610) in the present example, it should be understood that in other versions elongated shaft (1616) may be a separate component configured to be coupled to both coupler (1610) and probe assembly (20). In some other versions, elongated shaft (1616) may be associated with probe assembly (20) and thereby configured to couple with tissue sample holder assembly (1600). In the present example, elongated shaft (1616) is generally insertable into probe assembly (20).

Although not shown, it should be understood that coupler (1610) may include an operational coupling feature that is configured to securely attach coupler (1610) to probe assembly (20). By way of example only, the coupling feature may be in the form of a bayonet fitting, a threaded fitting, and/or etc. To assist an operator with coupling coupler (1610), ring-shaped body (1612) includes grips (1618) to enhance grip of ring-shaped body (1612) during locking and unlocking. Although the present example uses a proximal and distal movement to secure coupler (1610) to probe assembly (20), it should be understood that in other examples various alternative coupling features can be used as will be apparent to those of ordinary skill in the art in view of the teachings herein.

Track (1680) of coupler (1610) includes a top flange (1684) and a bottom flange (1686) extending across the length of track (1680), as best seen in FIG. 40. As will be described in greater detail below, flanges (1684, 1686) are sized and shaped to slidably receive and hold a portion of slidable member (1620) to coupler (1610). In other words, top flange (1684) is spaced apart from bottom flange (1686) in accordance with the size of a portion of slidable member (1620) such that track (1680) is configured to securely grasp slidable member (1620) between flanges (1684, 1686), as shown in FIG. 39.

Coupler (1610) includes an access port (1611) that defines an opening that is in fluid communication with the channel of elongated shaft (1616). Access port (1611) extends through track (1680) adjacent to a flexible detent (1682) and is in alignment with the cutter of needle (22). Access port (1611) is generally configured to receive tissue samples from the cutter of biopsy device (10) through elongated shaft (1616). As will be described in greater detail below, access port (1611) is generally sized to correspond to a series of openings (1648) of a manifold insert (1644) positioned within manifold (1640). As will also be described in greater detail below, manifold (1640) of slidable member (1620) can be translated to align access port (1611) with multiple chambers (1645) to thereby align the cutter of biopsy device (10) with a particular chamber (1645). As will be understood, when a particular chamber (1645) is aligned with flexible detent (1682), access port (1611) can then be used to provide that chamber (1645) access to the biopsy site through the cutter of biopsy device (10).

Coupler (1610) further includes a vacuum port (1613) that defines an opening that is configured to communicate with access port (1611) and elongate shaft (1616) when receiving tissue samples from biopsy device (10). Vacuum port (1613) is positioned proximate to access port (1611). As will be described in greater detail below, vacuum port (1613) is sized and shaped to correspond to an insert vacuum opening (1643) of a manifold insert (1644) and is configured to provide vacuum through manifold (1640) from probe assembly (20) to pull a tissue sample into tissue sample holder assembly (1600).

Slidable member (1620) is generally configured to receive tissue processing cassette (200) and position tissue processing cassette (200) relative to probe assembly (20) to thereby collect at least one tissue sample within tissue processing cassette (200). As best seen in FIG. 40, slidable member (1620) comprises a base (1622) extending around the perimeter of a proximal end of slidable member (1620) and a manifold (1640) integrally attached to base (1622). The width and/or shape of base (1622) is generally configured for receipt within track (1680) such that at least a portion of base (1622) is received within and slidably abuts track (1680) of tissue sample holder assembly (1600). In particular, base (1622) is slidably received between top flange (1684) and bottom flange (1686) such that flanges (1684, 1686) are sized and shaped to securely hold base (1622) of manifold (1640). In other words, the separation between top flange (1684) and bottom flange (1686) corresponds to the height of base (1622) such that track (1680) is configured to securely grasp slidable member (1620) within flanges (1684, 1686).

Manifold (1640) comprises a lower wall (1642), an upper wall (1646), and a pair of sidewalls (1650) extending between the lower wall (1642) and the upper wall (1646). Walls (1642, 1646, 1650) together define an irregularly shaped box that is configured to receive base (210) of tissue processing cassette (200). In this configuration, tissue processing cassette (200) is generally not capable of being received within tissue sample holder assembly (1600) in a closed position with lid (230) covering base (210). In particular, upper wall (1646) includes an angled portion (1647) intermediately extending between two flat portions of upper wall (1646). As will be described in greater detail below, angled portion (1647) of upper wall (1646) is configured to direct or divert tissue samples received within manifold (1640) toward tissue processing cassette (200). Walls (1642, 1646, 1650) further define an inner chamber (1652) that is sized and shaped to accommodate tissue processing cassette (200), while also providing fluid flow through manifold (1640).

Manifold (1640) further comprises a seal (1641) extending along the perimeter of an opening (1653) into inner chamber (1652), as best seen in FIG. 41. Seal (1641) is configured to form an airtight, compression fit against tissue processing cassette (200) when tissue processing cassette (200) is slidably inserted into manifold (1640). In this instance, seal (1641) is operable to abut against tissue processing cassette (200) to prevent any fluid and/or tissue samples from exiting tissue processing cassette (200) when positioned within manifold (1640). In other examples, seal (1641) is a rubber gasket to aid in the sealing of tissue processing cassette (200) relative to the exterior of manifold (1640). Various other suitable sealing features that may be included in manifold (1640) will be apparent to those of ordinary skill in the art.

Slidable member (1620) further includes a first raised connector (1623), a second raised connector (1624) and a third raised connector (1625) protruding upwardly from upper wall (1646). Raised connectors (1623, 1624, 1625) are spaced apart along upper wall (1646) along a single axis (not shown). Each raised connector (1623, 1624, 1625) is generally spaced from an adjacent raised connector (1626, 1624, 1625) such that raised connectors (1623, 1624, 1625) are collectively configured to selectively secure slidable member (1620) at a plurality of predetermined positions relative to coupler (1610). Although raised connectors (1623, 1624, 1625) are shown as single discrete parts that are integral with upper wall (1646), it should be understood that in other examples raised connectors (1623, 1624, 1625) can be separate parts, formed of more than one part, or a combination of both.

As will be described in greater detail below, each raised connector (1623, 1624, 1625) is generally configured to engage a flexible detent (1682) of track (1680) to selectively secure slidable member (1620) in a given predetermined position. As will also be described in greater detail below, raised connectors (1623, 1624, 1625) are generally configured to secure slidable member (1620) to coupler (1610) to thereby allow manifold (1640) to receive tissue samples axially relative to the longitudinal axis of access port (1611) and direct tissue samples downwardly into tissue processing cassette (200).

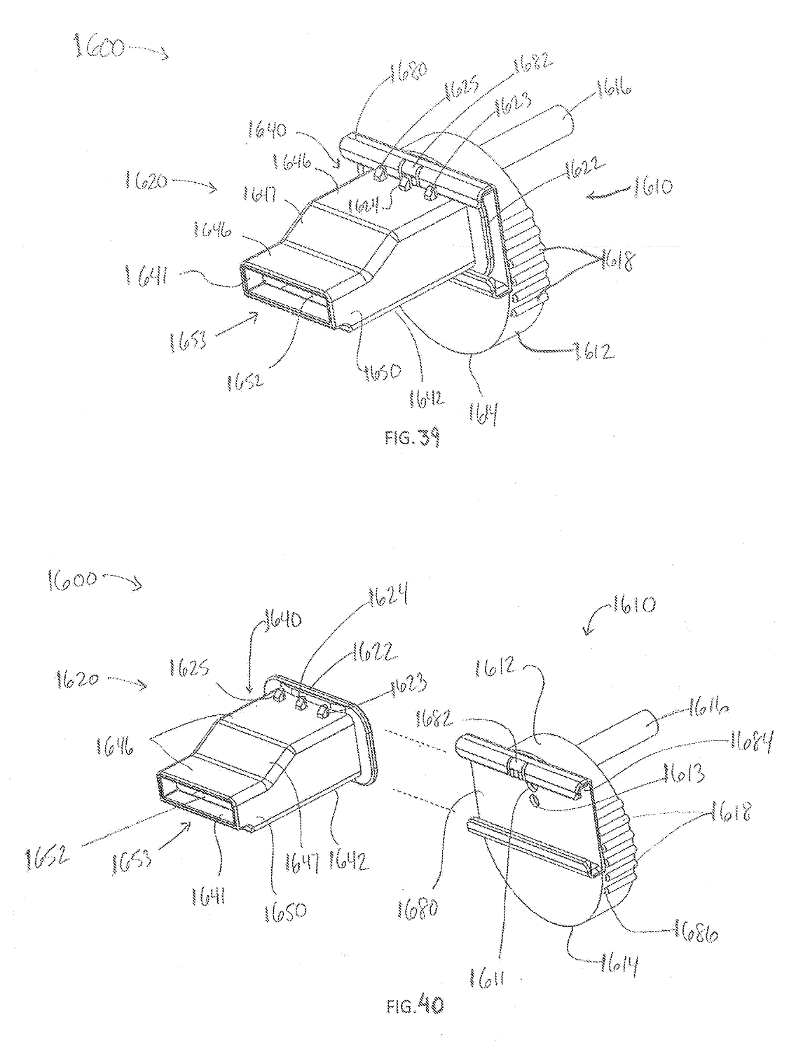

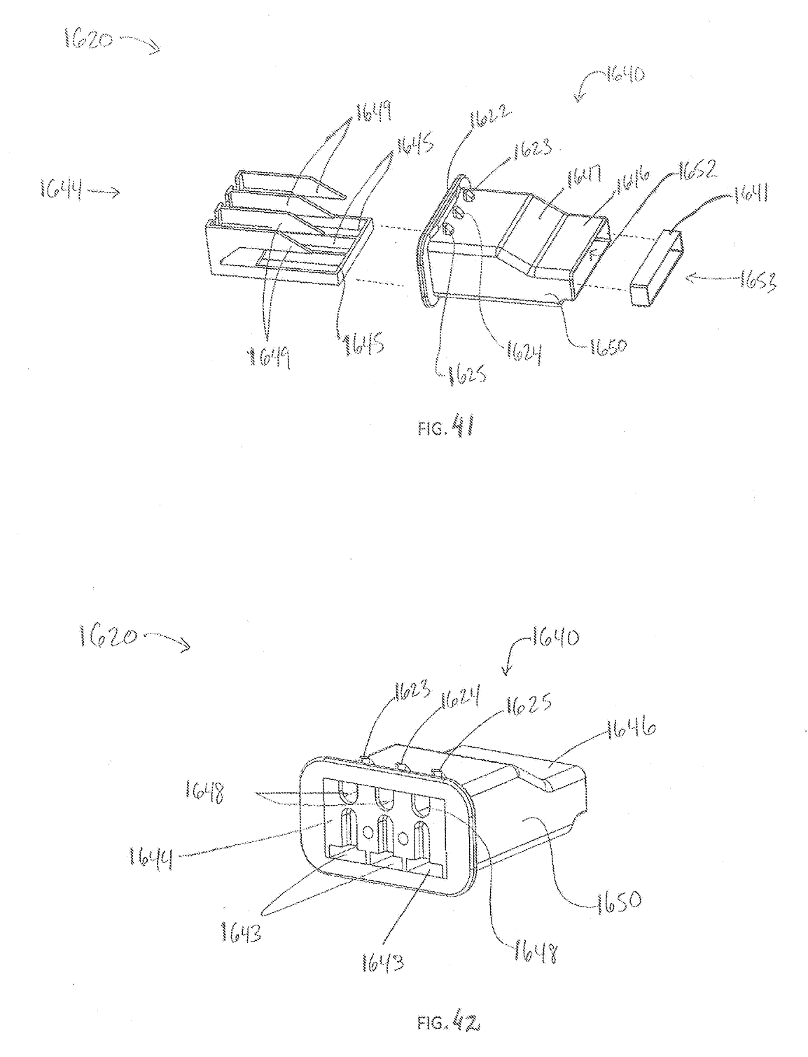

As best seen in FIG. 41, slidable member (1620) further includes a manifold insert (1644) slidably inserted into manifold (1640) at an end opposite of tissue processing cassette (200). Manifold insert (1644) comprises at least one chamber (1645) defined by a pair of walls (1649) such that manifold insert (1644) is configured to divide inner chamber (1652) into at least one chamber (1645) within manifold (1640). In other words, manifold insert (1644) is operable to define multiple chambers (1645) within manifold (1640) such that the tissue samples deposited within manifold (1640) may be separated to distinct chambers (1645) when received through slidable member (1620). In the present example, manifold insert (1644) comprises three chambers (1645) that are configured to correspond with the three raised connectors (1623, 1624, 1625) of manifold (1640). In other words, each chamber (1645) of manifold insert (1644) corresponds with the number of raised connectors (1623, 1624, 1625) provided on manifold (1640) such that chambers (1645) align with probe assembly (20) when a respective raised connector (1623, 1624, 1625) is engaged with flexible detent (1682) of track (1680). Each chamber (1645) communicates with probe assembly (20) via a respective fluid opening (1648) extending through manifold insert (1644), as best seen in FIG. 42. In particular, each fluid opening (1648) is defined between a pair of walls (1649) and are in fluid communication with a respective chamber (1645). Although not shown, it should be understood that manifold insert (1644) may comprise greater or fewer chambers (1645) as will be apparent to those of ordinary skill in the art.

Manifold insert (1644) further includes insert vent openings (1643) proximate to fluid openings (1648). In particular, each vent opening (1647) corresponds to a particular fluid opening (1648) on manifold insert (1644) such that a particular vent opening (1647) is configured to communicate with the respective chamber (1645) that is in communication with the corresponding vent opening (1648).

FIGS. 43-44 show a cassette insert (1690) configured to be inserted into tissue processing cassette (200). Similar to tissue processing cassette (200), cassette insert (1690) comprises a floor (1691) contained between a proximal wall (1692), a distal end (1693), and a pair of sidewalls (1694). Floor (1691) includes a plurality of vents (1695) extending between the pair of sidewalls (1694) to provide for fluid communication between cassette insert (1690) and tissue processing cassette (200). In other words, plurality of vents (1695) are generally associated with corresponding vents (224) of tissue processing cassette (200). As a result, cassette insert (1690) is in communication with tissue processing cassette (200) when a tissue sample extracted from the cutter of needle (22) is deposited within cassette insert (1690). This configuration generally promotes the flow of vacuum into tissue sample holder assembly (1600) through cassette insert (1690) and tissue processing cassette (200).

In accordance with the number of chambers (1645) included in manifold insert (1644), cassette insert (1690) is configured to comprise a corresponding number of channels (1698) that are sized and shaped to align with chambers (1645) of manifold insert (1644). Channels (1698) are defined by a pair of internal walls (1697) extending between proximal wall (1692) and distal end (1693). In the present example, as best seen in FIG. 44, cassette insert (1690) includes three channels (1698) in accordance with the three chambers (1645) of manifold insert (1644). Although not shown, it should be understood that cassette insert (1690) may include greater or fewer channels (1698) positioned therein depending on the number of chambers (1645) included in manifold insert (1644).

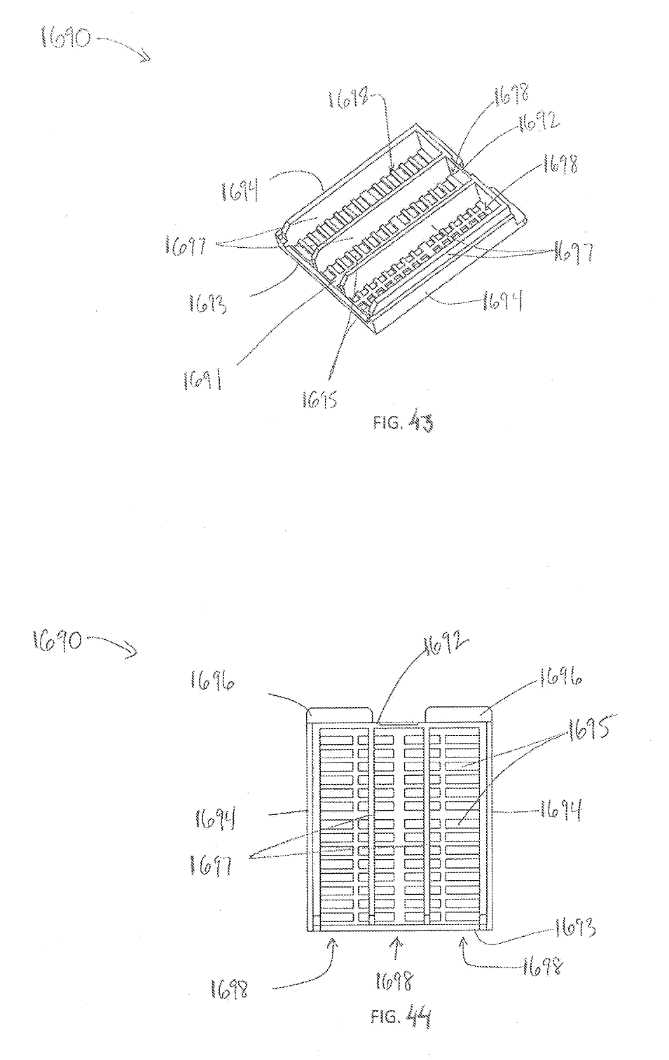

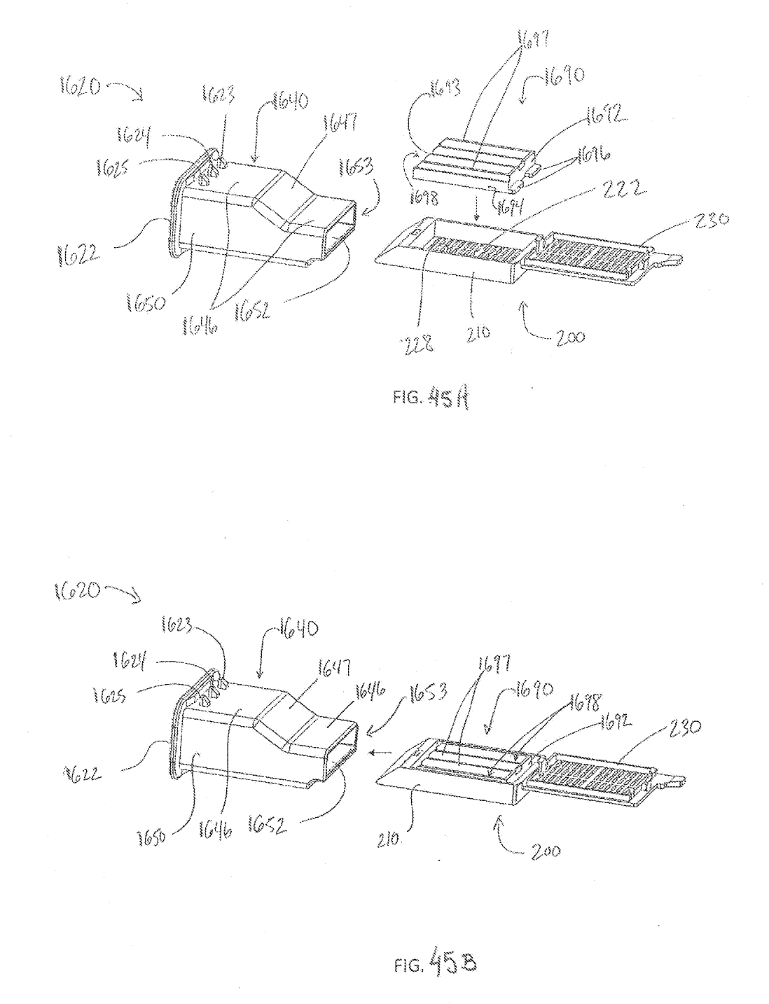

Cassette insert (1690) further includes a pair of manipulators (1696) that generally protrude outwardly from proximal wall (1692) to provide a gripping feature when cassette insert (1690) is inserted into tissue processing cassette (200). This facilitates inserting and removing cassette insert (1690) from tissue processing cassette (200) by providing a surface for an operator to grasp. Cassette insert (1690) is sized and shaped to be securely received within tissue processing cassette (200), specifically along floor (222) and within sample chamber (228), as seen in FIG. 45A. In particular, distal wall (1692) and manipulators (1696) of cassette insert (1690) are configured to abut against distal wall (212) of tissue processing cassette (200). In contrast, distal end (1693) of cassette insert (1690) is configured to abut against proximal wall (216) of tissue processing cassette (200). With cassette insert (1690) positioned within tissue processing cassette (200), manifold (1640) is configured to slidably receive the tissue processing cassette (200) through opening (1653) and into internal chamber (1652) as seen in FIG. 45B.

FIGS. 46-49 show an exemplary use of tissue sample holder assembly (1600) to collect tissue samples within tissue processing cassette (200). In particular, FIG. 46 shows the insertion of manifold insert (1644) into manifold (1640) and the separate insertion of processing cassette (200) into manifold (1640). As can be seen, chambers (1645) of manifold insert (1644) and channels (1698) of cassette insert (1690) become aligned when manifold insert (1644) and tissue processing cassette (200) are both received within manifold (1640). In this instance, tissue samples extracted by biopsy device (10) and transferred to tissue holder assembly (1600) will be selectively directed into a particular opening (1648) that is alignment with a particular chamber (1645), and subsequently deposited in a region of tissue processing cassette (200) defined by the channel (1698) of cassette insert (1690) that is in alignment with that chamber (1645). Unlike proximal wall (1692) of cassette insert (1690), distal end (1693) of cassette insert (1690) does not comprise a wall to allow for receipt of tissue samples into each channel (1698). In this configuration, distal end (1693) provides openings for channels (1698) to communicate with openings (1648) of chambers (1645) when selectively receiving a tissue sample therein. Although not shown, it should be understood that tissue processing cassette (200) may include cassette insert (1690) preinserted into sample chamber (228). Alternatively, or in addition, it should be understood that manifold (1640) may include manifold insert (1644) preinserted into inner chamber (1652).

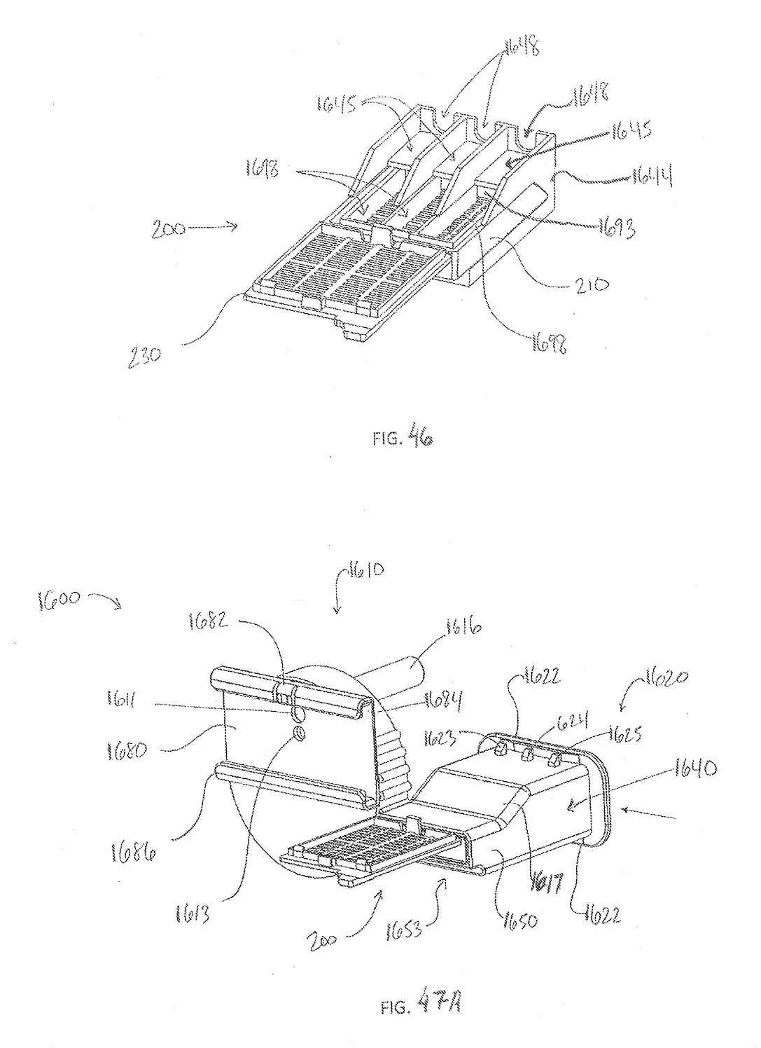

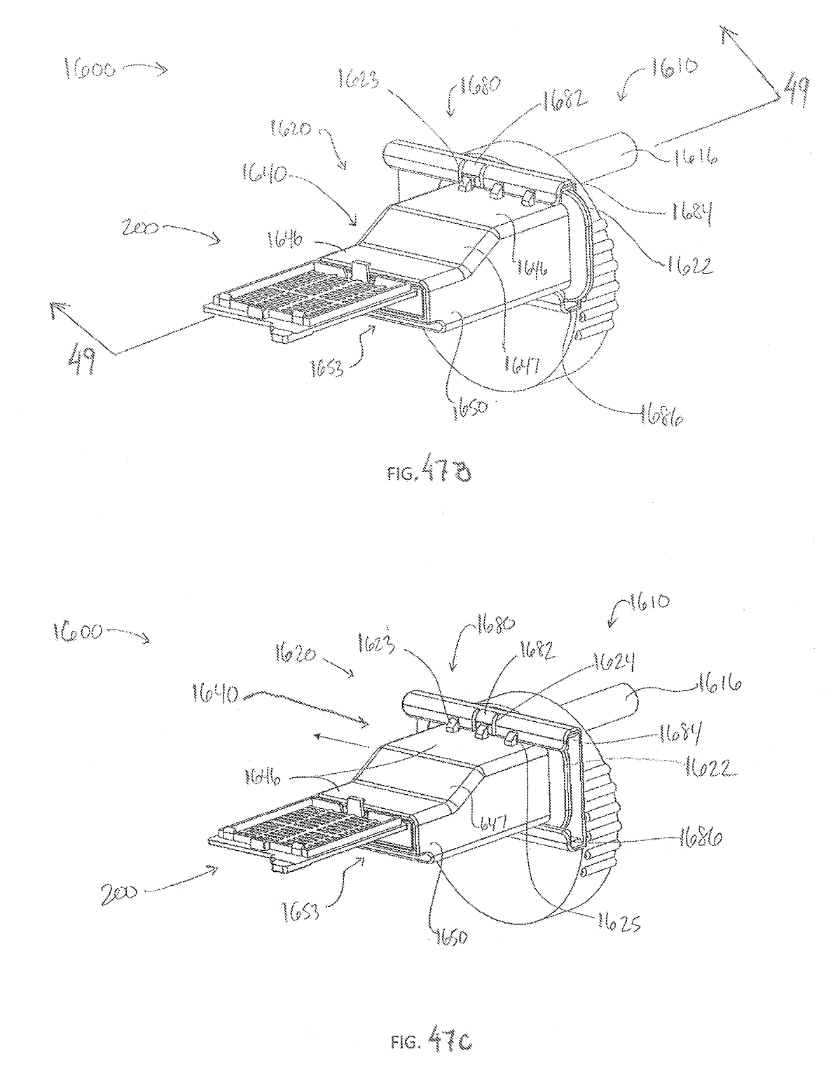

FIGS. 47A-47E show an exemplary progression of tissue sample holder assembly (1600) to fill each channel (1698) of cassette insert (1690) with at least one tissue sample. As can be seen, slidable member (1620) is slidably received by coupler (1610) by aligning base (1622) with track (1680) to thereby assemble tissue sample holder assembly (1600) to biopsy device (10). Although tissue processing cassette (200) is shown as being preassembled with manifold (1640) prior to the engagement of slidable member (1620) and coupler (1610), it should be understood that in other uses tissue processing cassette (200) may be inserted into manifold (1640) after tissue sample holder assembly (1600) is attached to probe assembly (20).

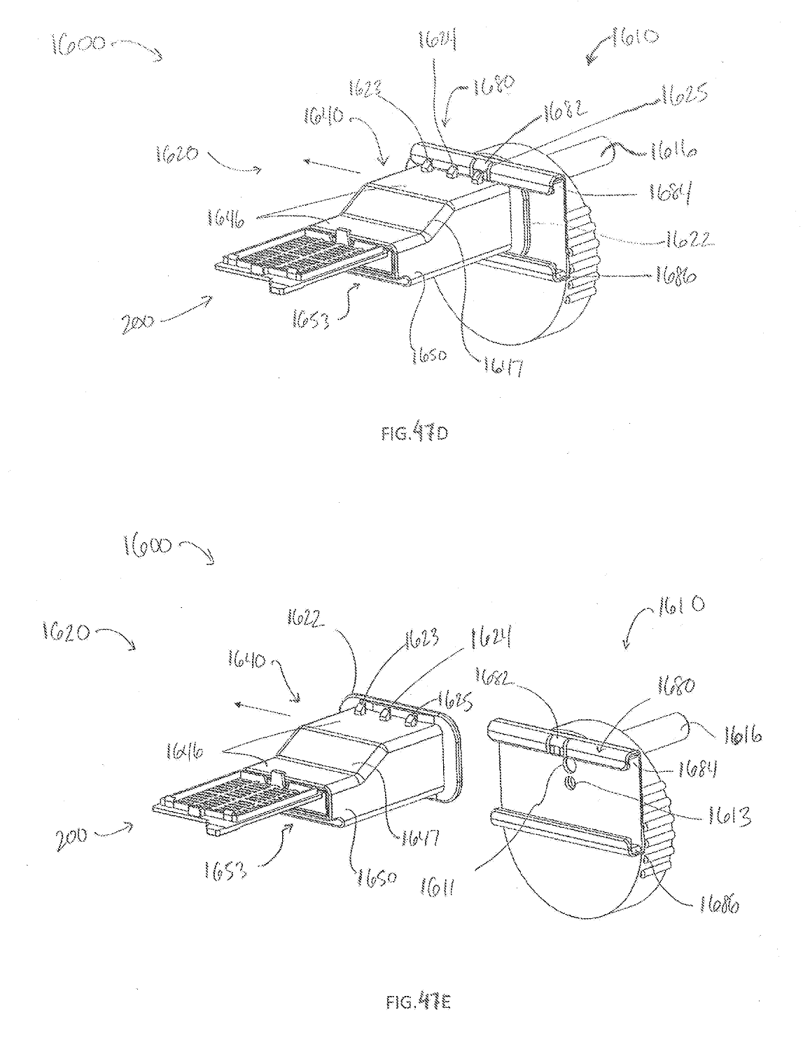

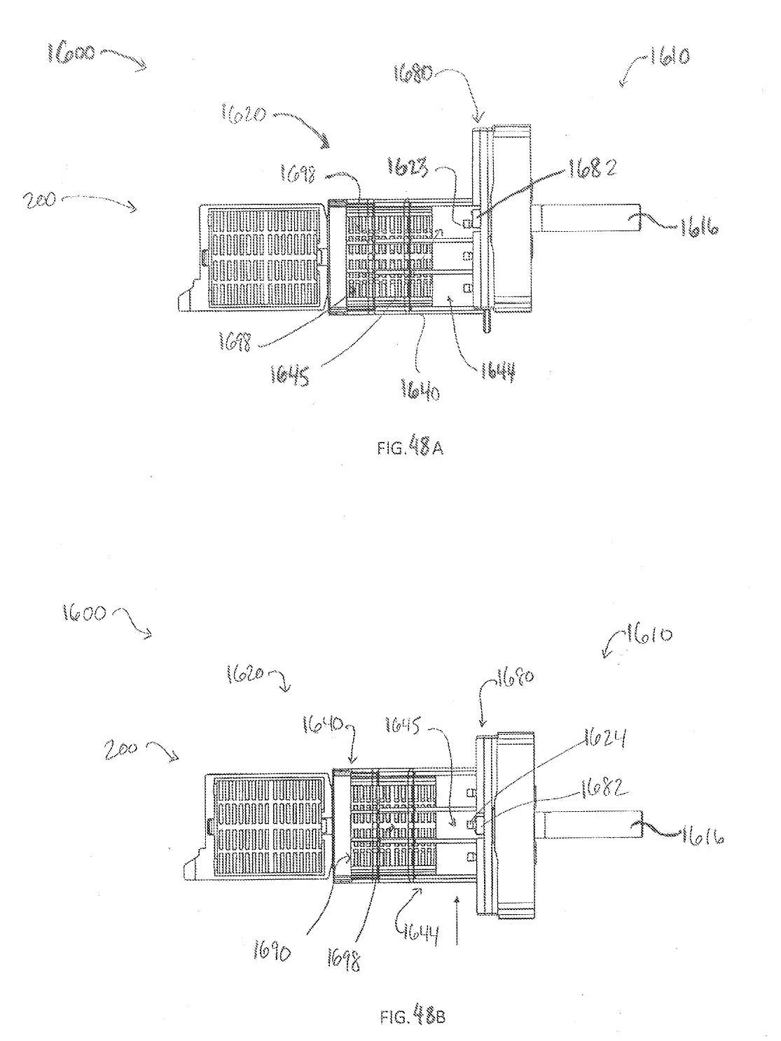

With base (1622) received between flanges (1684, 1686), slidable member (1620) is translated through track (1680) until first raised connector (1623) encounters flexible detent (1682). In this instance, as seen in FIG. 47B, slidable member (1620) becomes securely engaged with coupler (1610) through the coupling of first raised connector (1623) and flexible detent (1682) to place a particular chamber (1645) of manifold insert (1644) in communication with access port (1611). As described above, flexible detent (1682) and access port (1611) are generally aligned along a common axis such that aligning chamber (1645) with access port (1611) thereby provides fluid communication between chamber (1645) and the cutter of biopsy device (10) through elongated shaft (1616), as best seen in FIG. 48A. Accordingly, since cassette tray (1690) includes channels (1698) that are in corresponding alignment with chambers (1645) of manifold insert (1644), the translation of slidable member (1620) simultaneously positions a respective channel (1698) in communication with the cutter of biopsy device (10) to accommodate for the receipt of tissue samples within tissue processing cassette (200).

As best seen in FIG. 47C, applying a predetermined force upon slidable member (1620) provides for the disengagement of first raised connector (1623) and flexible detent (1682) such that slidable member (1620) translates further along track (1680) until second raised connector (1624) encounters flexible detent (1682). In this instance, as best seen in FIG. 48B, a subsequent chamber (1645) of manifold insert (1644) becomes aligned with access port (1611) and elongated shaft (1616). Any tissue samples extracted by the cutter of biopsy device (10) will be transferred to this subsequent chamber (1645), and the corresponding channel (1698) of cassette insert (1690) that is in alignment with the subsequent chamber (1645).

As seen in FIG. 47D, an operator may apply a second instance of force upon slidable member (1620) to disengage the second raised connector (1623) from flexible detent (1682) to thereby translate slidable member (1620) further along track (1680) until third raised connector (1625) engages flexible detent (1682). As seen in FIG. 48C, with slidable member (1620) securely engaged to coupler (1610) at third raised connector (1625), a third chamber (1645) of manifold insert (1644) is in alignment with access port (1611) and elongated shaft (1616). In this instance, a third channel (1698) of cassette insert (1690) is aligned to receive tissue samples from the cutter of biopsy device (10).

Although not shown, it should be understood that slidable member (1620) may be slidably coupled to coupler (1610) in an opposite order, where third raised connector (1625) initially encounters flexible detent (1682), rather than first raised connector (1623). It will also be apparent to those of ordinary skill in the art that an operator may cease slidably translating slidable member (1620) along track (1680) once a sufficient number of tissue samples (90) have been deposited within tissue processing cassette (200). In other words, although tissue sample holder assembly (1600) is described above as being used to collect a single tissue sample (90) in each channel (1698) of cassette insert (1690), it should be understood that in some uses it may be desirable to only collect samples (90) into one or more specific channels (1698) of cassette insert (1690). Accordingly, in some uses slidable member (1620) may be translated to skip some channels (1645) of manifold insert (1644). Similarly, it should be understood that an operator is not required to further advance sliable member (1620) through each raised connector (1623, 1624, 1625) iteration. Rather, an operator may simply retract slidable member (1620) in the same direction that slidable member (1620) was initially inserted into track (1680). It should also be understood that tissue processing cassette (200) may be removed from manifold (1640) at any point during the engagement of slidable member (1620) and coupler (1610) such that slidable member (1620) is not required to be disengaged from coupler (1610) prior to removing tissue processing cassette (200) from within inner chamber (1652). In addition, slidable member (1620) may remain at a given position for multiple sample collection sequences to collect more than a single tissue sample (90) within one or more channels (1698) of cassette insert (1690).

FIG. 49 shows a path (80) of a tissue sample (90) traveling through elongated shaft (1616) towards coupler (1610) until arriving at access port (1611). In this instance, with a particular chamber (1645) of manifold insert (1644) aligned with access port (1611), tissue sample (90) enters manifold (1640) of slidable member (1620) until encountering angled portion (1647). In other words, it should be understood that when tissue sample (90) is received within manifold (1640), tissue sample (90) does not proceed directly into channel (1698) of cassette insert (1690). Instead, tissue sample (90) first enters the area of chamber (1645) defined by manifold insert (1644) and upper wall (1646). The transition of the interior of upper wall (1646) to angled portion (1647) then directs tissue sample (90) downwardly into the corresponding channel (1698) of cassette insert (1690). Thus, it should be understood that angled portion (1647) of upper wall (1646) acts as a tissue sample deflector, director, or channeler to direct tissue sample (90) into tissue processing cassette (200) after tissue sample (90) is received through opening (1648) of chamber (1645). Accordingly, although angled portion (1647) is shown as having a specific angle and/or geometry, it should be understood that the particular configuration of angled portion (1647) may vary based on a number of considerations such as the positioning of each opening (1648) relative to manifold insert (1644), the velocity of tissue sample (90) transport from elongated shaft (1616), the size of tissue samples (90), the gage size of needle (22), and/or etc.

With tissue sample (90) deposited within tissue processing cassette (200), channel (1698) of cassette insert (1690) isolates tissue sample (90) from any other tissue samples (90) previously deposited, or to be deposited, within tissue processing cassette (200). Seal (1641) forms an airtight seal against tissue processing cassette (200) while tissue processing cassette (200) is contained within manifold (1640). In this instance, seal (1641) abuts a proximal portion of tissue processing cassette (200) and prevents any fluid and/or tissue samples (90) from exiting tissue processing cassette (200).

Within inner chamber (1652), lower wall (1642) includes a vacuum opening (1656) and a vacuum chamber (1654). Vacuum opening (1656) extends along lower wall (1642) in parallel alignment with tissue processing cassette (200) when inserted into inner chamber (1652). Vacuum opening (1656) does not extend along the entire longitudinal length of tissue processing cassette (200) to thereby provide support for tissue processing cassette (200) when received within manifold (1640). Vacuum chamber (1654) similarly extends along lower wall (1642) and is in fluid communication with tissue processing cassette (200) through vents (224) and vacuum opening (1656). Vacuum chamber (1654) is in communication with vacuum opening (1656) to communicate vacuum from probe assembly (20) into tissue processing cassette (200). Vacuum enters manifold (1640) through vacuum opening (1656) that is in communication with vacuum port (1613) of probe assembly (20). In particular, vacuum port (1613) of probe assembly (20) provides vacuum through insert vacuum opening (1643) of manifold insert (1644), which is in communication with vacuum chamber (1656). Next, vacuum travels through vacuum chamber (1654) and upwardly through vents (224) of tissue processing cassette (200). Vacuum then travels through corresponding vents (1695) of cassette insert (1690) and into inner chamber (1652) of manifold (1640). In this instance, the vacuum is now in communication with opening (1648) of the particular chamber (1645) that is currently aligned with access port (1611), thereby effectively being positioned in communication with the cutter of needle (22). Vacuum is then used to pull tissue sample (90) through the cutter of needle (22) and into the corresponding chamber (1645) of manifold insert (1644).

Once a sample is received within channel (1698) of cassette insert (1690), slidable member (1620) is translated to a subsequent position along track (1680), shown in FIGS. 47A-47E. This translation indexes the next successive chamber (1645) and channel (1698) to receive a tissue sample (90) therein by being in communication with corresponding features of probe assembly (20) as similarly described above. In this position, another tissue sample (90) may be collected in tissue processing cassette (200) corresponding to the selected chamber (1645) of manifold insert (1644). Once channels (1690) of cassette insert (1690) are filled with a tissue sample (90) as desired by an operator, an operator may next desire to perform certain analysis on the collected tissue samples (90). To perform analysis on the collected tissue samples (90), an operator first removes tissue processing cassette (200) from manifold (1640) of slidable member (1620). At this stage, tissue processing cassette (200) may be manipulated for a visual inspection of each tissue sample (90). In addition, tissue processing cassette (200) may be placed in a procedure room x-ray unit to perform a preliminary analysis of the tissue samples (90). If an operator is not satisfied with the results at this stage, undesirable tissue samples (90) may be discarded and the same tissue processing cassette (200) with cassette insert (1690) positioned therein may be inserted back into manifold (1640) of slidable member (1620) for collection of additional tissue samples (90). Alternatively, an entirely new tissue processing cassette (200) and cassette insert (1690) may be placed into manifold (1640) of slidable member (1620) for collection of additional tissue samples (90).

Once tissue samples (90) are collected to the satisfaction of an operator, the operator may desire to transport tissue samples (90) to a pathology laboratory. At this stage, an operator closes lid (230) with base (210) and, if applicable, may mark or place a label onto tissue processing cassette (200) to ensure chain of custody through the workflow as will be apparent to those of ordinary skill in the art. Alternatively, in some uses, tissue processing cassette (200) may already be labeled at this stage. For instance, in some uses tissue processing cassette (200) may be labeled at the beginning of the biopsy procedure before collecting any tissue samples (90). Alternatively, in some uses tissue processing cassette (200) may be prelabeled with a bar code, QR code, or another computer readable medium. Where such computer readable mediums are used, tissue processing cassette (200) may be scanned at various stages to associate the computer readable medium with the patient. This may include multiple scans throughout the procedure such as before the biopsy procedure, after collection of tissue samples, after procedure room x-ray, and/or etc.

Once chain of custody has been established, the combination of tissue processing cassette (200) and cassette insert (1690) may be inserted into jar (160) described above. As described above, jar (160) may be filled with a fluid such as formalin to preserve the collected tissue samples during transport and/or storage. Although tissue processing cassette (200) and cassette insert (1690) is described herein as being used with the same jar (160) described above, it should be understood that other alternative jars or containers may be used for transport and/or storage of tissue processing cassette (200) and cassette insert (1690). For instance, in some examples jar (160) may be replaced with a container of a variety of shapes and sizes. In other examples, tissue processing cassette (200) itself may be used to transport tissue samples (90). Of course, in such examples structures of tissue processing cassette (200) such as vents (224) can be closed so that tissue processing cassette (200) can hold fluids such as formalin.

After the combination of tissue processing cassette (200) and cassette insert (1690) is inserted into jar (160), jar (160) may be transported to the pathology laboratory as shown in FIG. 7 and described above. The collected tissue samples (90) may then be processed in accordance with the workflow (300) shown in FIG. 7. Since the original tissue processing cassette (200) was used with tissue sample holder assembly (1600), it should be understood that the steps of workflow (300) remain applicable in this instance, such as straining the collected samples as represented by box (350) and placing the collected samples into a tissue processing cassette (200) as represented by box (370). In addition, it should be understood that at any one or more of the steps depicted in FIG. 7, an operator may interact with a label to confirm chain of custody of the collected tissue samples. By way of example only, this may include scanning computer readable mediums associated with the label, confirming information on the label with information on jar (160) or other components, or confirming information on the label with patient files.

B. Alternative Retractable Tissue Sample Holder Assembly

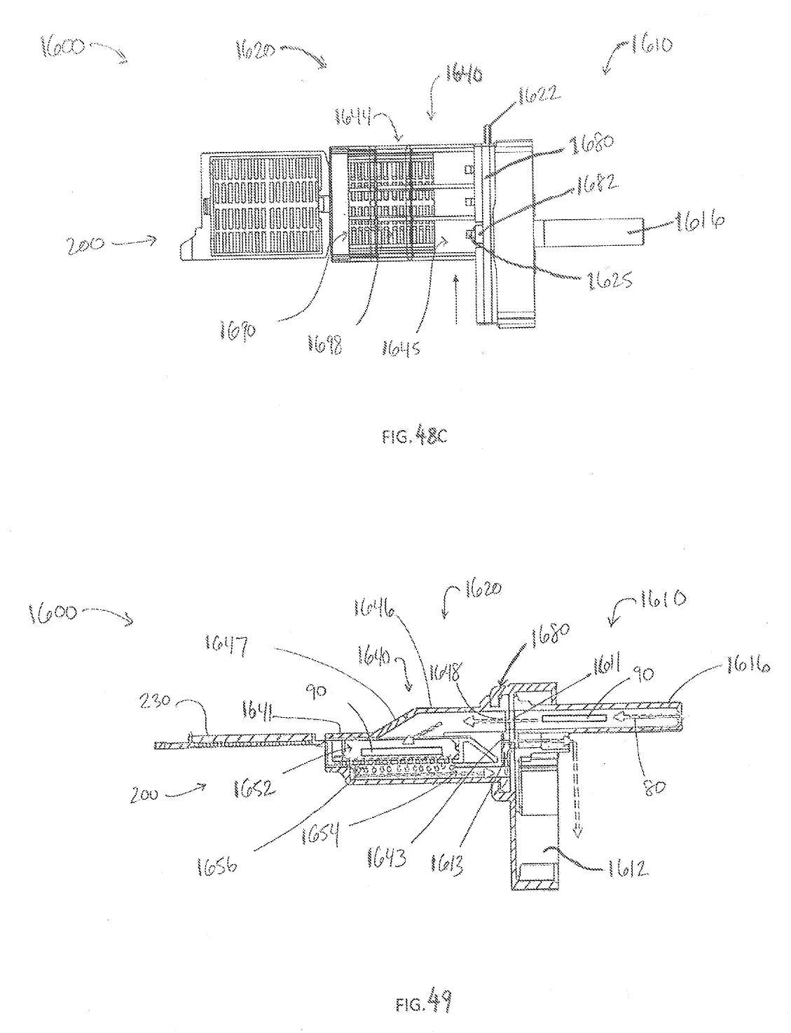

FIGS. 50-51 show an exemplary alternative tissue sample holder assembly (1700) that may be used with biopsy device (10) in lieu of tissue sample holder assembly (40, 1600) described above. Tissue sample holder assembly (1700) comprises a coupler (1710), a slidable member (1720), and a track (1780). Coupler (1710) comprises a generally ring-shaped body (1712) with a closed proximal end (1714), an elongated shaft (1716), and a plurality of grips (1718). Closed proximal end (1714) is configured to engage at least a portion of probe assembly (20) to seal coupler (1710) to probe assembly (20). In addition, closed proximal end (1714) is configured to fixedly secure coupler (1710) to probe assembly (20) to thereby permit translation of slidable member (1720) relative to coupler (1710) within track (1780). As will be described in greater detail below, this translation permits tissue processing cassette (200) to be moved relative to probe assembly (20) so that at least one tissue sample can be collected within floor (222).

Elongated shaft (1716) defines an internal channel that is configured to be in fluid communication with needle (22) of biopsy device (10) such that shaft (1716) is operable to receive tissue samples extracted from a patient. Thus, elongated shaft (1716) is received by and connected to probe assembly (20) to form a connection with the cutter of biopsy device (10) to selectively pull tissue samples through probe assembly (20) and into tissue sample holder assembly (1700). Although elongated shaft (1716) is described as being an integral component with coupler (1710) in the present example, it should be understood that in other versions elongated shaft (1716) may be a separate component configured to be coupled to both coupler (1710) and probe assembly (20). In some other versions, elongated shaft (1716) may be associated with probe assembly (20) and thereby configured to couple with tissue sample holder assembly (1700). In the present example, elongated shaft (1716) is generally insertable into probe assembly (20).

Although not shown, it should be understood that coupler (1710) may include an operational coupling feature that is configured to securely attach coupler (1710) to probe assembly (20). By way of example only, the coupling feature may be in the form of a bayonet fitting, a threaded fitting, and/or etc. To assist an operator with coupling coupler (1710), ring-shaped body (1712) includes grips (1718) to enhance grip of ring-shaped body (1712) during locking and unlocking. Although the present example uses an insertion movement to secure coupler (1710) to probe assembly (20), it should be understood that in other examples various alternative coupling features can be used as will be apparent to those of ordinary skill in the art in view of the teachings herein.

Track (1780) of coupler (1710) includes a top flange (1784) and a bottom flange (1786) extending across the length of track (1780), as best seen in FIG. 51. As will be described in greater detail below, flanges (1784, 1786) are sized and shaped to slidably receive and hold a portion of manifold (1740) to coupler (1710). In other words, top flange (1784) is spaced apart from bottom flange (1786) in accordance with the size of a portion of manifold (1740) such that track (1780) is configured to securely grasp slidable member (1720) between flanges (1784, 1786), as shown in FIG. 50.

Coupler (1710) includes an access port (1711) that defines an opening that is in fluid communication with the channel of elongated shaft (1716). Access port (1711) extends through track (1780) and is in alignment with the cutter of needle (22). Access port (1711) is generally configured to receive tissue samples from the cutter of biopsy device (10) through elongated shaft (1716). As will be described in detail below, manifold (1740) of slidable member (1720) can be translated to align access port (1711) with an opening in the manifold (1740) to thereby align the cutter of biopsy device (10) with tissue processing cassette (200). In particular, when a manifold access opening (1743) is aligned with access port (1711), elongate shaft (1716) can then be used to provide manifold (1740) access to the biopsy site through the cutter of biopsy device (10). Coupler (1710) further includes a vacuum port (1713) that defines an opening that is configured to communicate with access port (1711) and elongate shaft (1716) when receiving tissue samples from biopsy device (10). As will be described in greater detail below, vacuum port (1713) is sized and shaped to correspond to a manifold vacuum opening (1744) of manifold (1740) and is configured to provide vacuum through manifold (1740) from probe assembly (20) to pull a tissue sample into tissue sample holder assembly (1700).

Slidable member (1720) is generally configured to receive tissue processing cassette (200) and position tissue processing cassette (200) relative to probe assembly (20) to thereby collect at least one tissue sample within tissue processing cassette (200). As best seen in FIG. 51, slidable member (1720) comprises a base (1722) extending around the perimeter of a proximal end of slidable member (1720) and a manifold (1740) integrally attached to base (1722). The width and/or shape of base (1722) is generally configured to be received within track (1780) such that at least a portion of base (1722) slidably abuts track (1780) of tissue sample holder assembly (1700). In particular, base (1722) is slidably received between top flange (1784) and bottom flange (1786) such that flanges (1784, 1786) are sized and shaped to securely hold base (1722) of manifold (1740). In other words, the separation between top flange (1784) and bottom flange (1786) corresponds to the height of base (1722) such that track (1780) is configured to securely grasp slidable member (1720) within flanges (1784, 1786).

Manifold (1740) comprises a lower wall (1742), an upper wall (1746), and a pair of sidewalls (1748) extending between the lower wall (1742) and the upper wall (1746). Walls (1742, 1746, 1748) together define an irregularly shaped box that is configured to receive tissue processing cassette (200) such that tissue processing cassette (200) is capable of being received within tissue sample holder assembly (1700) in a closed position with lid (230) covering base (210). Upper wall (1746) includes an angled profile extending between base (1722) and an opening (1750). As will be described in greater detail below, the angled profile of upper wall (1746) is configured to accommodate for the separation of lid (230) from base (210) within manifold (1740) to allow tissue samples to be received within tissue processing cassette (200) contained therein. As best seen in FIG. 53, walls (1742, 1746, 1748) further define an inner chamber (1752) that is sized and shaped to accommodate tissue processing cassette (200), while also providing fluid flow through manifold (1740). Manifold (1740) further comprises a seal (1741) extending along the perimeter of opening (1750) into inner chamber (1752). Seal (1741) is configured to form an airtight, compression fit against tissue processing cassette (200) when tissue processing cassette (200) is slidably inserted into manifold (1740). In this instance, seal (1741) is operable to abut against tissue processing cassette (200) to prevent any fluid and/or tissue samples from exiting tissue processing cassette (200) when positioned within manifold (1740). In other examples, seal (1741) is a rubber gasket to aid in the sealing of tissue processing cassette (200) relative to the exterior of manifold (1740). Various other suitable sealing features that may be included in manifold (1740) will be apparent to those of ordinary skill in the art.

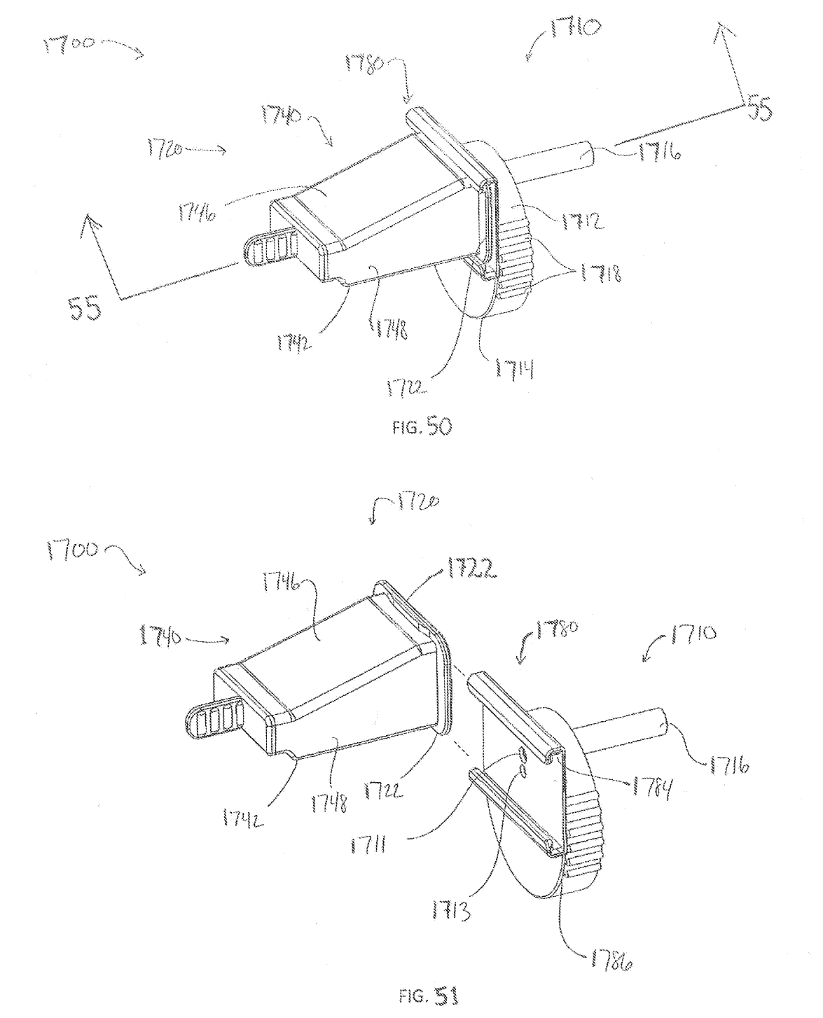

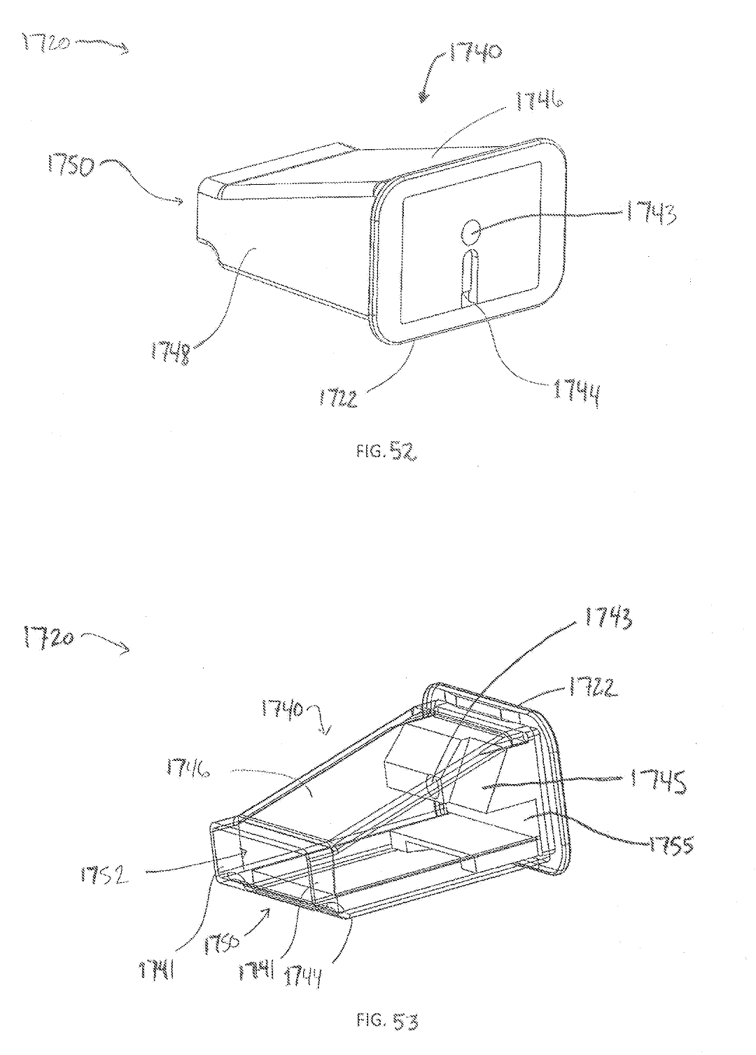

Unlike slidable member (1620) described above, slidable member (1720) is generally configured to freely slide along track (1780) such that slidable member (1720) is not securely coupled or engaged to coupler (1710) by any fastening mechanisms, such as raised connectors or flexible detents. Rather, slidable member (1720) is selectively translated relative to coupler (1710) to thereby allow manifold (1740) to receive tissue samples when a manifold access opening (1743) is axially aligned to the longitudinal axis of access port (1711) to thereby permit tissue samples to be received by tissue processing cassette (200). As best seen in FIG. 52, manifold access opening (1743) is sized, shaped, and positioned along manifold (1740) in accordance with the size, shape, and position of access port (1711) such that manifold access opening (1743) is able to align with access port (1711) when manifold (1740) is slidably translated in track (1780). As further seen in FIG. 53, manifold access opening (1743) extends into inner chamber (1752) such that fluid communication between elongate shaft (1716) and inner chamber (1752) is established when manifold access opening (1743) is aligned with access port (1711). Manifold (1740) further includes a manifold vacuum opening (1744) proximate to manifold access opening (1743). As will be described in greater detail below, manifold vacuum opening (1744) is configured to allow for vacuum within inner chamber (1752) of manifold (1740).

As best seen in FIG. 53, slidable member (1720) further includes a manifold ramp (1745) at an end opposite of opening (1750). Manifold ramp (1745) is proximate to manifold access opening (1743) and is positioned within inner chamber (1752) to align with manipulator (244) when tissue processing cassette (200) is received within manifold (1740). In particular, manifold ramp (1745) is configured to engage manipulator (244) as tissue processing cassette (200) is slidably advanced into inner chamber (1752). Upon encountering ramp (1745), manipulator (244) is configured to drive upward along ramp (1745) thereby causing lid (230) to separate from base (210) of tissue processing cassette (200). In other words, manifold ramp (1745) is operable to open tissue processing cassette (200) within manifold (1740) by engaging manipulator (244) and separating lid (230) from base (210) as tissue processing cassette (200) is advanced into manifold (1740). Slidable member (1720) further includes a cavity (1755) at an end opposite of opening (1750) and positioned relatively below ramp (1745). As will be described in greater detail below, cavity (1755) is generally sized and shaped to receive a distal end of a drawer (1748) to thereby securely retain tissue processing cassette (200) within manifold (1740).

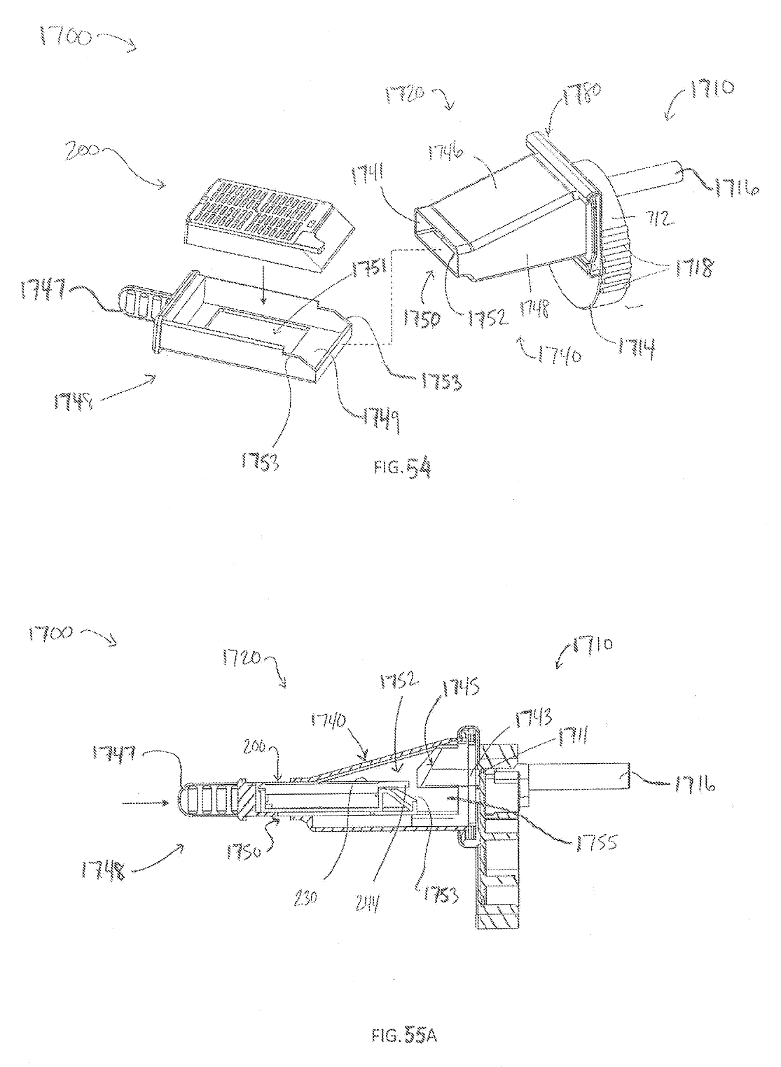

Tissue processing cassette (200) is slidably inserted into manifold (1740) by means of a drawer (1748), as seen in FIG. 54. Drawer (1748) is sized and shaped to receive tissue processing cassette (200) within a receiving tray (1749). Drawer (1748) is further sized and shaped to be received within inner chamber (1752) of manifold (1740) such that drawer (1748) is configured to slidably insert tissue processing cassette (200) through opening (1750) and into inner chamber (1752), as seen in FIG. 54. Drawer (1748) includes a handle (1747) protruding proximally from receiving tray (1749) at a proximal end. Handle (1747) is configured to allow for selective maneuvering of drawer (1748) into and out of manifold (1740). Although handle (1747) is shown as an extending tab with a semi-circular profile, it should be understood that handle (1747) may comprise various configurations or shapes as will be apparent to those of ordinary skill in the art. Additionally, handle (1747) may include gripping features thereon configured to enhance the gripping strength of drawer (1748) for ease in maneuverability.

At a distal end opposite of handle (1747), drawer (1748) includes an engagement mechanism (1753) in the form of a tapered configuration. Engagement mechanism (1753) is sized and shaped to be received underneath ramp (1745) of manifold (1740) such that engagement mechanism (1753) is configured to securely retain drawer (1748) within inner chamber (1752) when received underneath ramp (1745) in a cavity (1755). Drawer (1748) further includes an aperture (1751) positioned along receiving tray (1749). As will be described in greater detail below, aperture (1751) is sized and shaped to correspond to a vent opening (1756) of manifold (1740) to thereby allow probe assembly (20) to create a vacuum through manifold (1740) to pull tissue samples into tissue sample holder assembly (1700). In the present example, manipulator (244) of tissue processing cassette (200) is positioned at an end opposite of handle (1747) when inserted into drawer (1748) such that manipulator (244) is positioned in direct alignment with ramp (1745) as drawer (1748) and tissue processing cassette (200) is advanced into inner chamber (1752). Although not shown, it should be understood that cassette insert (1690) described above may be inserted into tissue processing cassette (200) prior to placing tissue processing cassette (200) into drawer (1748).

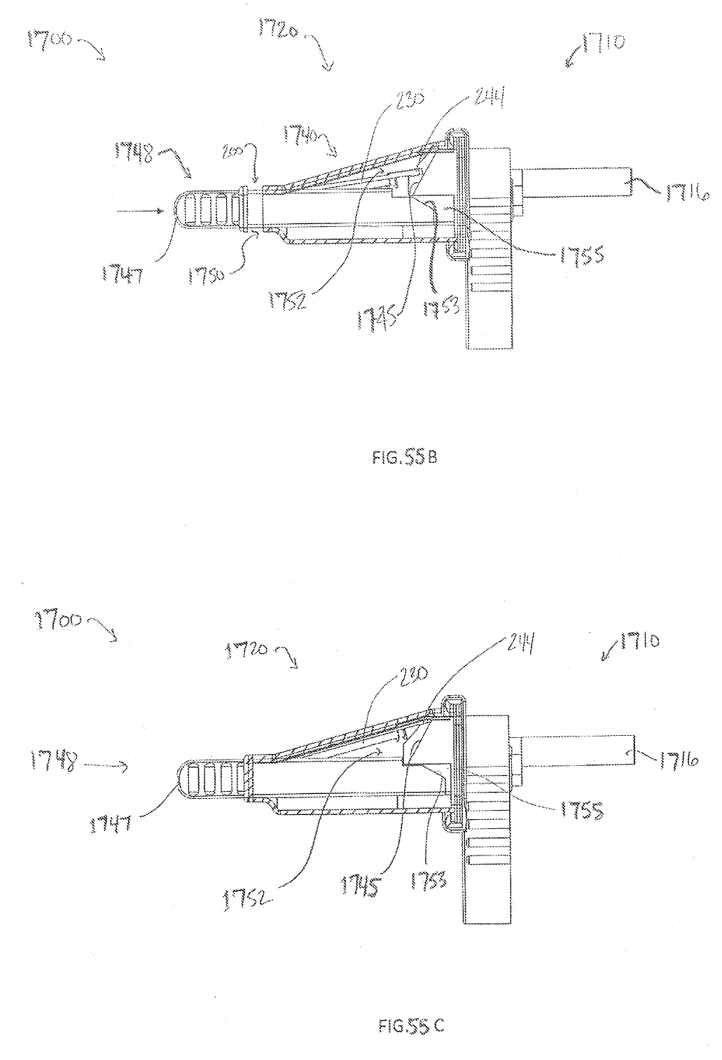

FIGS. 54-57 show an exemplary use of tissue sample holder assembly (1700) to collect tissue samples within tissue processing cassette (200). In particular, FIG. 54 shows the insertion of tissue processing cassette (200) into receiving tray (1749) of drawer (1748). With tissue processing cassette (200) received within drawer (1748), drawer (1748) may subsequently be advanced into manifold (1740). FIGS. 55A-55C show an exemplary progression of tissue sample holder assembly (1700) to fill tissue processing cassette (200) with at least one tissue sample. As can be seen, with slidable member (1720) slidably received by coupler (1710) by aligning base (1718) with track (1780), tissue sample holder assembly (1700) is effectively assembled to biopsy device (10). Although tissue processing cassette (200) is described as being preassembled within drawer (1748) prior to the engagement of slidable member (1720) and coupler (1710), it should be understood that in other uses tissue processing cassette (200) and drawer (1748) may be inserted into manifold (1740) after slidable member (1720) is attached to coupler (1710).

With base (1722) received within track (1780), slidable member (1720) is translated through track (1780) until manifold access opening (1743) aligns with access port (1711). At any point prior to or after positioning of slidable member (1720), tissue processing cassette (200) can be inserted into manifold (1740) via drawer (1748). In particular, as seen in FIG. 55A, drawer (1748) is advanced through opening (1750) and into inner chamber (1752). As described above, manipulator (244) encounters ramp (1745) such that lid (230) is forcibly opened as the handle (1747) is continuously exerted inwardly relative to manifold (1740), as seen in FIG. 55B.

As manipulator (244) rides up ramp (1745), lid (230) extends upwardly towards upper wall (1746) of manifold (1740) until manipulator (244) reaches the end of ramp (1745), as seen in FIG. 55C. In this instance, manipulator (244) is securely engaged to a portion between upper wall (1746) and ramp (1745) such that drawer (1748) is securely fastened to manifold (1740) to thereby inhibit the unrestricted removal of drawer (1748) from inner chamber (1752). With lid (230) in a partially opened state relative to base (210), slidable member (1740) is operable to transfer tissue samples received from access port (1711) into tissue processing cassette (200), as seen in FIG. 56. In other words, with manifold access opening (1743) being generally aligned along a common axis with elongate shaft (1716), a tissue processing cassette (200) is able to receive a tissue sample extracted by the cutter of biopsy device (10) and transported through access port (1711). Subsequent translation of slidable member (1720) offsets the alignment of manifold access opening (1743) with elongate shaft (1716) thereby ceasing communication between tissue processing cassette (200) and the cutter of biopsy device (10). Applying a predetermined force upon slidable member (1720) provides for the translation of manifold (1740) along track (1780) such that slidable member (1720) eventually becomes disengaged from coupler (1710).

Although not shown, it should be understood that slidable member (1720) may be slidably coupled to coupler (1710) in an opposite order, where slidable member (1720) initially encounters track (1780) at an opposite end than that depicted or described above. It will also be apparent to those of ordinary skill in the art that an operator may cease slidably translating slidable member (1720) along track (1780) once a sufficient number of tissue samples (90) have been deposited within tissue processing cassette (200). Similarly, it should be understood that an operator is not required to further advance sliable member (1720) completely through track (1780) prior to removing drawer (1748) and tissue processing cassette (200). Rather, an operator may simply remove drawer (1748) by grasping handle (1747) at any point during the engagement of slidable member (1720) and coupler (1710) such that slidable member (1720) is not required to be disengaged from coupler (1710) prior to removing tissue processing cassette (200) from within inner chamber (1752).

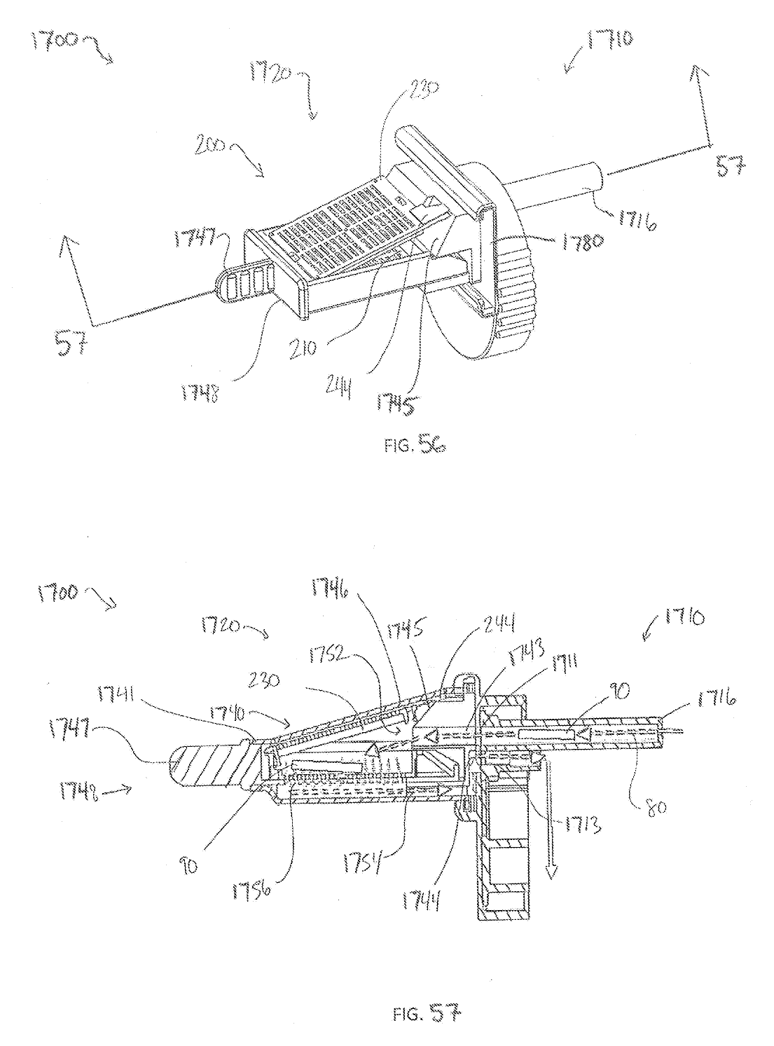

FIG. 57 shows a path (80) of a tissue sample (90) traveling through elongated shaft (1716) towards coupler (1710) until arriving at access port (1711). In this instance, with manifold access opening (1743) aligned with access port (1711), tissue sample (90) enters manifold (1740) of slidable member (1720) until encountering lid (230) of tissue processing cassette (200). In other words, it should be understood that when tissue sample (90) is received within manifold (1740), tissue sample (90) does not proceed directly into floor (222). Instead, tissue sample (90) first enters inner chamber (1752) by encountering lid (230), which is supported by upper wall (1746). The angled profile of upper wall (1746) similarly positions lid (230) at an angle such that tissue sample (90) is directed downward towards floor (222). Thus, it should be understood that lid (230) acts as a tissue sample deflector, director, or channeler to direct tissue sample (90) into tissue processing cassette (200) after tissue sample (90) is received through manifold access opening (1743). Accordingly, although upper wall (1746) is shown as having a specific angle and/or geometry, it should be understood that the particular configuration of upper wall (1746) may vary based on a number of considerations such as the positioning of manifold opening (1746) relative to manifold (1740), the velocity of tissue sample (90) transport from elongated shaft (1716), the size of tissue samples (90), the gage size of needle (22), and/or etc.

With tissue sample (90) deposited within tissue processing cassette (200), seal (1741) forms an airtight seal against tissue processing cassette (200) while tissue processing cassette (200) is contained within manifold (1740). In this instance, seal (1741) abuts a proximal portion of tissue processing cassette (200) and prevents any fluid and/or tissue samples (90) from exiting tissue processing cassette (200). Within inner chamber (1752), lower wall (1742) includes a vacuum opening (1756) and a vacuum chamber (1754). Vacuum opening (1756) extends along lower wall (1742) in parallel alignment with tissue processing cassette (200) when inserted into inner chamber (1752). Vacuum opening (1756) does not extend along the entire longitudinal length of tissue processing cassette (200) to thereby provide support for tissue processing cassette (200) when received within manifold (1740). Vacuum chamber (1756) similarly extends along lower wall (1742) and is in fluid communication with tissue processing cassette (200) through vents (224) and vacuum opening (1754). Vacuum chamber (1756) is in communication with vacuum opening (1754) to communicate vacuum from probe assembly (20) into tissue processing cassette (200). Vacuum enters manifold (1740) through vacuum opening (1754) that is in communication with vacuum port (1713) of probe assembly (20) through manifold vacuum opening (1744). Next, vacuum travels through vacuum chamber (1756) and upwardly through vents (224) of tissue processing cassette (200) and into inner chamber (1752) of manifold (1740). In this instance, the vacuum is now in communication with manifold opening (1754) that is currently aligned with access port (1711), thereby effectively being positioned in communication with the cutter of needle (22). Vacuum is then used to pull tissue sample (90) through the cutter of needle (22) and into the inner chamber (1752).

Once a sample is received within tissue processing cassette (200), slidable member (1720) is translated along track (1780) to offset the alignment of manifold opening (1754) and access port (1711), shown in FIG. 51. This translation prevents any subsequent tissue samples (90) from being received within manifold (1740) and thereby mixed with the original tissue samples (90) deposited within tissue processing cassette (200). At this point, an operator may retract drawer (1748) from slidable member (1720) to thereby remove tissue processing cassette (200) from inner chamber (1752) of manifold (1740). In this instance, an operator grasps handle (1747) and pulls proximally, away from coupler (1710) and probe assembly (20), to disengage the secured coupling of manipulator (244) and ramp (1745). In this instance, manipulator (244) rides downwardly along ramp (1745) and drawer (1748) exits manifold (1740) from opening (1750). As similarly seen in FIG. 55B, lid (230) of tissue processing cassette (200) beings to transition as manipulator (244) is translated on ramp (1745). In this instance, lid (230) transitions from an open state distal to base (210) (see FIG. 55C) towards a closed state where lid (230) is proximate to base (210) (see FIG. 55A). With manipulator (244) no longer positioned along ramp (1745), lid (230) abuts against base (210) but remains slightly ajar relative to base (210) until drawer (1748) is retracted completely from inner chamber (1752) such that manipulator (244) encounters seal (1741). In this instance, the compression fit created by seal (1741) at opening (1750) serves to compress lid (230) and base (210) towards each other as tissue processing cassette (200) is passed through opening (1750). As drawer (1748) and tissue processing cassette (200) are withdrawn from manifold (1740), lid (230) becomes securely fastened to base (210) such that tissue processing cassette (200) is in a closed state once drawer (1748) is completely removed from inner chamber (1752).

In this instance, with drawer (1748) withdrawn from slidable member (1720) and tissue processing cassette (200) no longer contained within manifold (1740), an operator may remove tissue processing cassette (200) from receiving tray (1749), as seen in FIG. 54. With tissue processing cassette (200) filled with the desired number of tissue samples (90), an operator may continue to perform certain analysis on the collected tissue samples (90) as similarly described above.

C. Alternative Rotatable Tissue Sample Holder Assembly

In some instances, it may be desirable to use tissue processing cassette (200) in connection with biopsy device (10) through a tissue sample holder assembly that is configured to receive tissue processing cassette (200) but which also comprises fewer removable components than tissue sample holder assembly (1600, 1700) described above. Providing a tissue sample holder assembly that allows for tissue samples to be collected directly into tissue processing cassette (200) in a simplified manner than that of tissue sample holder assembly (1600, 1700) may be beneficial to simplify the extraction and deposit of tissue samples into tissue processing cassette (200). As described above, tissue sample holder assembly (40) is generally configured to be completely removable from probe assembly (20) of biopsy device (10). Thus, a suitable alternative tissue sample holder assembly may be used in lieu of tissue holder assembly (40, 1600, 1700), provided certain vacuum and tissue sample collection couplings remain consistent between the suitable alternative tissue sample holder assembly and tissue sample holder assembly (40). As similarly discussed above, it may be further beneficial to configure the alternative tissue sample holder assembly to be used with cassette insert (1690) such that tissue processing cassette (200) is able to collect multiple tissue samples therein while maintaining the multiple tissue samples in isolation from each other.

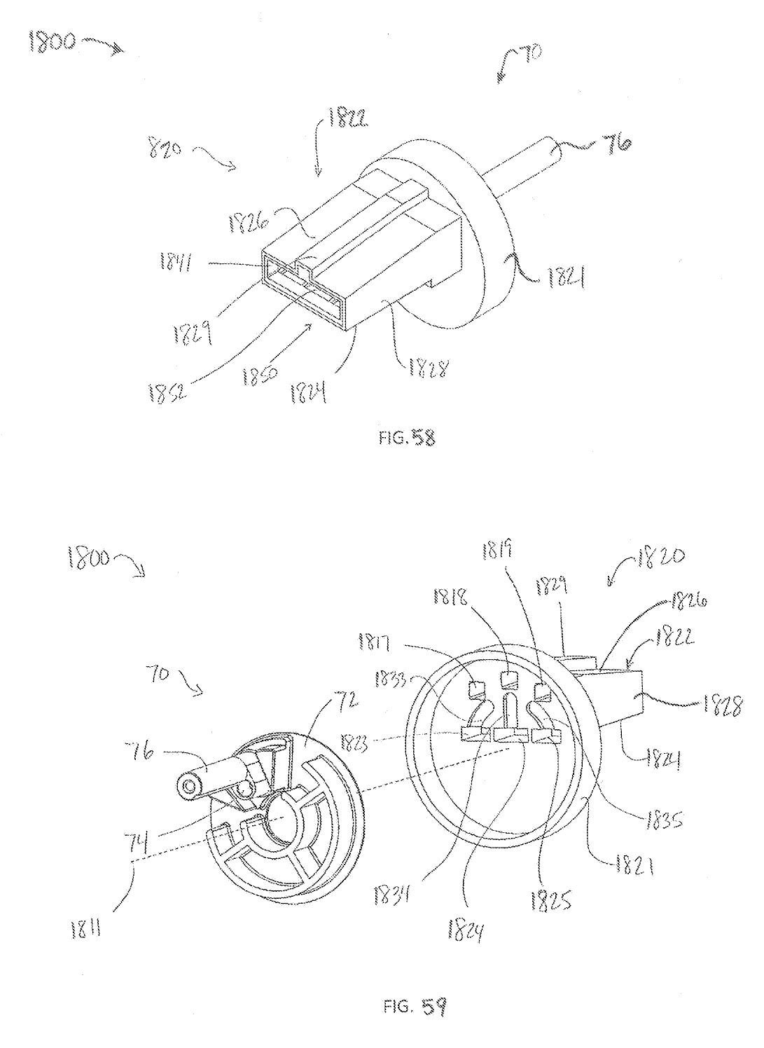

FIGS. 58-59 show an exemplary alternative tissue sample holder assembly (1800) that may be used with biopsy device (10) in lieu of tissue sample holder assembly (40, 1600, 1700) described above. Tissue sample holder assembly (1800) comprises a rotatable member (1820) as shown in FIG. 58. Rotatable member (1820) comprises a cylindrically-shaped body (1821) and a manifold (1822), where body (1821) is configured to couple to a probe seal (70) of probe assembly (20). Probe seal (70) comprises a generally circular-shaped body (92), a vent port (94), and an elongated shaft (96). As best seen in FIG. 59, circular-shaped body (92) is received within cylindrically-shaped body (1821) of rotatable member (1820) to thereby seal rotatable member (1820) to probe seal (70) of probe assembly (20). In other words, probe seal (70) is configured to rotatably secure rotatable member (1820) to probe assembly (20). Rotatable member (1820) is configured to rotate relative to probe seal (70) such that cylindrically-shaped body (1821) of rotatable member (1820) is rotatably coupled to circular-shaped body (92) of probe seal (70). In this instance, rotatable member (1820) is operable to rotate relative to probe assembly (20). Thus, rotatable member (1820) is configured to rotate within circular-shaped body (92) of probe seal (70) about a common axis (1811). As will be described in greater detail below, this rotation permits tissue processing cassette (200) to rotate relative to probe assembly (20) so that at least one tissue sample can be collected within each channel (1698) of cassette insert (1690). As will be further described below, vent port (94) is configured to provide a vacuum in rotatable member (1820) from probe assembly (20).

Elongated shaft (76) defines an internal channel that is configured to be in fluid communication with needle (22) of biopsy device (10) such that shaft (76) is operable to receive tissue samples extracted from a patient. Thus, elongated shaft (76) is received by and connected to probe assembly (20) to form a connection with the cutter of biopsy device (10) to selectively pull tissue samples through probe assembly (20) and into tissue sample holder assembly (1800). In this configuration, elongated shaft (76) is integral with probe assembly (20) such that probe seal (70) is securely fixed to probe assembly (20) and thus not rotatable relative to probe assembly (20). Although probe seal (70) is described as being an integral component of probe assembly (20), it should be understood that tissue sample holder assembly (1800) may include a coupler as similarly described above in lieu of probe seal (70) of the present example.

Rotatable member (1820) is generally configured to receive tissue processing cassette (200) and position tissue processing cassette (200) relative to probe assembly (20) to thereby collect a tissue sample within each channel (1698) cassette insert (1690) that is included in tissue processing cassette (200). As best seen in FIG. 58, cylindrically-shaped body (1821) is sized and shaped to be received on circular-shaped body (72) to thereby couple rotatable member (1820) to probe seal (70). The width and/or shape of manifold (1822) is generally configured to receive base (210) of tissue processing cassette (200) such that lid (230) of tissue processing cassette (200) extends outwardly from within manifold (1822) of tissue sample holder assembly (1800). Manifold (1822) comprises a lower wall (1824), an upper wall (1826), and a pair of sidewalls (1828) extending between the lower wall (1824) and the upper wall (1846). Walls (1824, 1826, 1828) together define an irregularly shaped box that is configured to receive tissue processing cassette (200). In particular, upper wall (1826) includes a protruding portion (1829) extending upwardly from upper wall (1826) and along the length of upper wall (1826) from cylindrically-shaped body (1821) to an opening (1850).

As will be described in greater detail below, protruding portion (1829) is sized and shaped to account for a channel within manifold (1822) that provides communication between access port (1818) and an inner chamber (1852). Inner chamber (1852) is defined by walls (1824, 1826, 1828) and is sized and shaped to accommodate tissue processing cassette (200) therein, while also providing fluid flow through manifold (1822).

Manifold (1822) further comprises a seal (1841) extending along the perimeter of opening (1850). Seal (1841) is configured to form an airtight, compression fit against tissue processing cassette (200) when tissue processing cassette (200) is slidably inserted into manifold (1822). In this instance, seal (1841) is operable to abut against tissue processing cassette (200) to prevent any fluid and/or tissue samples from exiting tissue processing cassette (200) when positioned within manifold (1822). In other examples, seal (1841) is a rubber gasket to aid in the sealing of tissue processing cassette (200) relative to the exterior of manifold (1822). Various other suitable sealing features that may be included in manifold (1822) will be apparent to those of ordinary skill in the art.

As seen in FIG. 59, rotatable member (1820) includes three access ports (1817, 1818, 1819) that define an opening configured to provide fluid communication between the channel of elongated shaft (76) and manifold (1822). Access ports (1817, 1818, 1819) include respective channels that commence at the opening (1817, 1818, 1819) formed at the distal end of manifold (1822) and extend through manifold (1822) at three distinct locations. In particular, the particular positions of access ports (1817, 1818, 1819) correspond to distinct rotational orientations of rotatable member (1820) relative to probe seal (70). In other words, access ports (1817, 1818, 1819) are tissue receiving channels that are orientated about manifold (1822) at various predetermined locations that correspond to a respective portion within inner chamber (1852) of manifold (1822). Access ports (1817, 1818, 1819) are integrally formed within manifold (1822) and are configured to have elongated channels extending through manifold (1822) that are separate from each other such that a particular access port (1817, 1818, 1819) is not in fluid communication with another access port (1817, 1818, 1819).

Access ports (1817, 1818, 1819) are generally configured to individually receive tissue samples from the cutter of biopsy device (10) through elongated shaft (76) such that only one access port (1817, 1818, 1819) maintains fluid communication with elongated shaft (76) at a given moment depending on the particular rotational alignment of rotatable member (1820) relative to probe seal (70). As will be described in greater detail below, manifold (1822) of rotatable member (1820) can be rotated to align an access port (1817, 1818, 1819) with elongate shaft (76) of probe seal (70) to thereby establish fluid communication between the cutter of biopsy device (10) and a particular channel (1698) of cassette insert (1690) that is aligned with that access port (1817, 1818, 1819). In other words, each respective access port (1817, 1818, 1819) aligns with a particular channel (1698) of cassette insert (1690) contained within tissue processing cassette (200) such that access port (1817, 1818, 1819) can be used to provide a particular channel (1698) access to the biopsy site through the cutter of biopsy device (10) when rotated to align with elongate shaft (76) of probe seal (70).

Rotatable member (1820) further includes vacuum openings (1823, 1824, 1825) extending into inner chamber (1852) and positioned below access ports (1817, 1818, 1819), respectively. Vacuum openings (1823, 1824, 1825) include a respective channel (1833, 1834, 1835) extending upwardly from vacuum opening (1823, 1824, 1825) and toward a corresponding access port (1817, 1818, 1819). As seen in FIG. 59, channels (1833, 1834, 1835) are integral with rotatable member (1820) such that channels (1833, 1834, 1835) are cut into the distal end of rotatable member (1820) at an adequate depth to provide vacuum communication between vacuum openings (1824, 1825, 1826) and vent port (74). In particular, channels (1833, 1834, 1835) are generally configured to correspond with the position of vent port (74) of probe seal (70) when rotatable member (1820) is rotated to align a particular access port (1817, 1818, 1819) with elongate shaft (76). In other words, channels (1833, 1834, 1835) and vacuum openings (1823, 1824, 1825) rotate simultaneously with rotatable member (1820) such that when an access port (1817, 1818, 1819) arrives in alignment with elongate shaft (76) a corresponding channel (1833, 1834, 1835) aligns with vent port (74) of probe seal (70). In this instance, channel (1833, 1834, 1835) establishes communication between vent port (74) of probe seal (70) and vacuum opening (1823, 1824, 1825) of rotatable member (1820) to thereby allow for a vacuum to be formed within manifold (1822).

As seen, channels (1834, 1834, 1835) may comprise a straight, angular, or irregular shape and/or alignment in correspondence to the respective locations of access ports (1817, 1818, 1819) and vacuum openings (1824, 1825, 1826). The varying configurations of channels (1833, 1834, 1835) allow vacuum openings (1823, 1824, 1825) to be aligned in a straight line along the distal end of rotatable member (1820). As will be described in greater detail below, the straight alignment of vacuum openings (1823, 1824, 1825) are configured to correspond with the positions of relative portions of tissue processing cassette (200) contained within manifold (1822). As further seen in FIG. 59, upper wall (1826) of rotatable member (1820) is generally shaped at a downward angle relative to a longitudinal axis of elongate shaft (76) and access ports (1817, 1818, 1819) such that rotatable member (1820) is configured to direct tissue samples downwardly towards tissue processing cassette (200) when received by the channels of access ports (1817, 1818, 1819).

Unlike manifold (1640) described above, manifold (1822) of rotatable member (1820) does not include a manifold insert comprising multiple inner walls configured to divide inner chamber (1852) into multiple chambers. Rather, inner chamber (1852) is effectively divided into separate regions through the inclusion of cassette insert (1690) in tissue processing cassette (200) when tissue processing cassette (200) is inserted into manifold (1822). The number of access ports (1817, 1818, 1819) on rotatable member (1820) corresponds to the number of channel (1698) in cassette insert (1690) such that any tissue samples inserted into manifold (1822) by a particular access port (1817, 1818, 1819) will be delivered and deposited within a respective channel (1698). The angled configuration of upper wall (1826) of manifold (1822) is configured to minimize any space between upper wall (1826) and inner walls (1697) of cassette insert (1690) to thereby ensure any tissue samples transferred to manifold (1822) through a particular access port (1817, 1818, 1819) will be deposited in the appropriate channel (1698) of cassette insert (1690).

In the present example, manifold (1822) comprises three access ports (1817, 1818, 1819) and vacuum openings (1823, 1824, 1825) that are configured to correspond with the three channels (1698) of cassette insert (1690). In other words, each access port (1817, 1818, 1819) of manifold (1822) corresponds with the number of channels (1698) provided by cassette insert (1690) in tissue processing cassette (200) such that the channels of access ports (1817, 1818, 1819) align with between the inner walls (1697) of cassette insert (1690). Each channel (1698) communicates with probe assembly (20) via a respective access port (1817, 1818, 1819) extending through manifold (1822). Although not shown, it should be understood that manifold (1822) may comprise greater or fewer access ports (1817, 1818, 1819) and vacuum openings (1823, 1824, 1825) as will be apparent to those of ordinary skill in the art.

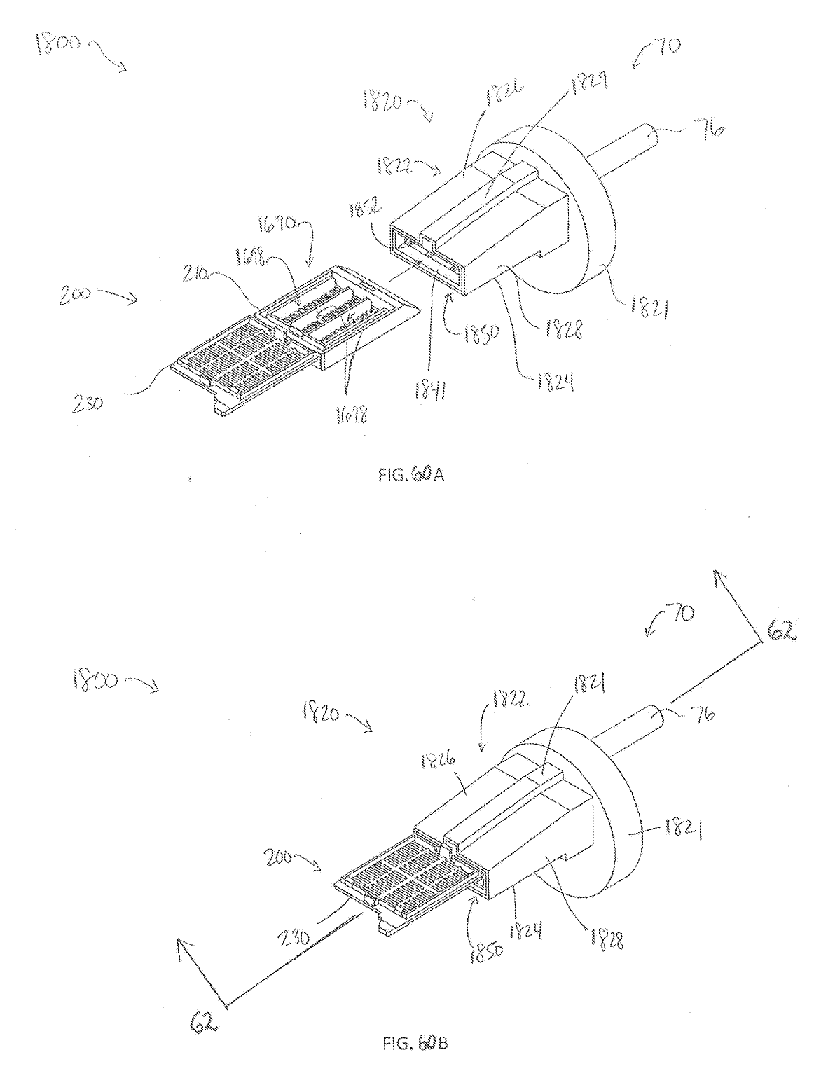

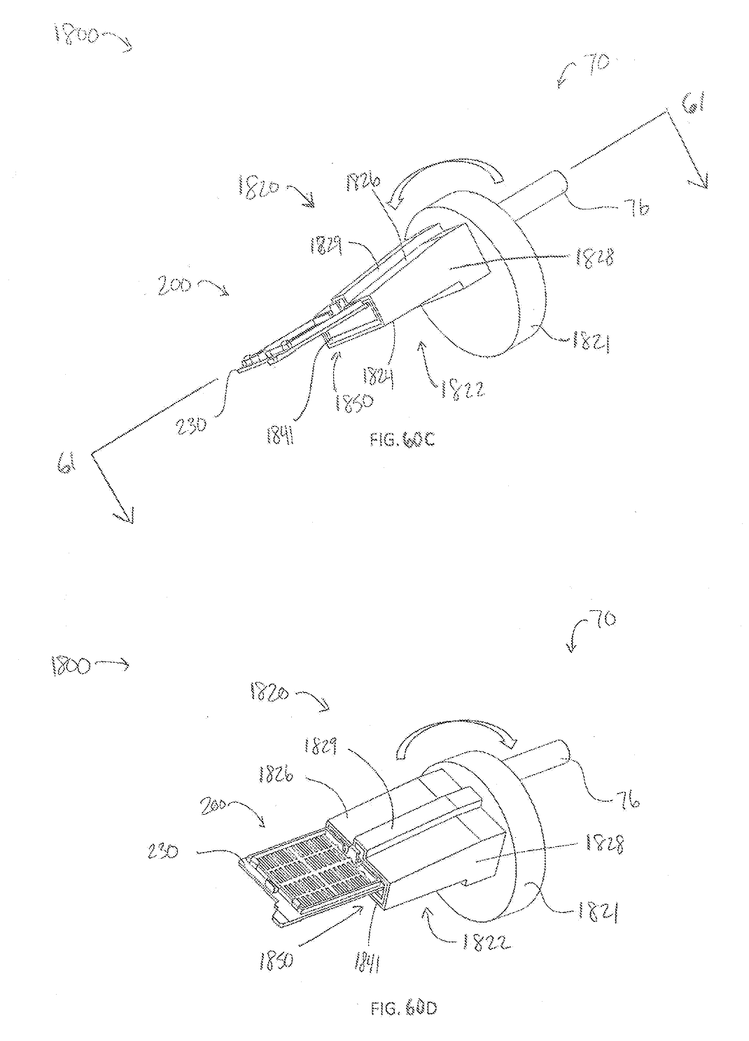

FIGS. 60A-62 show an exemplary use of tissue sample holder assembly (1800) to collect tissue samples within tissue processing cassette (200). In particular, FIGS. 60A-60D show an exemplary progression of tissue sample holder assembly (1800) to fill each channel (1698) of cassette insert (1690) with at least one tissue sample. As can be seen, tissue processing cassette (200) is in an open configuration with base (210) separated from lid (230) and cassette insert (1690) received within base (210), as seen in FIG. 60A. After cassette insert (1690) is inserted into base (210), tissue processing cassette (200) is slidably advanced through opening (1850) of inner chamber (1852) and into manifold (1822). In this instance, as seen in FIG. 60B, rotatable member (1820) is in an orientation where access port (1818) is generally aligned along a common axis with elongate shaft (76) of probe seal (70) such that a respective channel (1698) of cassette insert (1690) that is in alignment with access port (1818) is in fluid communication with the cutter of biopsy device (10). Accordingly, any tissue samples extracted by an operator with the rotatable member (1820) maintained in this current orientation will result in the tissue samples being deposited within the middle channel (1698) of the three channels (1698) of cassette insert (1690). Accordingly, the rotation of rotatable member (1820) in either a left or right direction, relative to probe assembly (20) and probe seal (70), simultaneously positions a different channel (1698) in communication with the cutter of biopsy device (10).

As best seen in FIG. 60C, applying a predetermined force upon rotatable member (1820) provides for the rotation of manifold (1822) and tissue processing cassette (200) contained therein such that rotatable member (1820) realigns relative to probe assembly (20) until access port (1817) couples with elongate shaft (76). In this instance, as further seen in FIG. 61, the left-most channel (1698) of cassette insert (1690) becomes aligned with elongate shaft (76). Thus, any tissue samples extracted by the cutter of biopsy device (10) will be transferred to this subsequent channel (1698) that is in alignment with access port (1817). Alternatively, or subsequently, as seen in FIG. 60D, an operator may apply a force upon rotatable member (1820) to realign manifold (1822) relative to probe assembly (20) to thereby rotate tissue processing cassette (200) further until access port (1819) engages elongate shaft (76). With rotatable member (1820) securely engaged to the channel of elongate shaft (76) at access port (1819), the right-most channel (1698) of cassette insert (1690) is in alignment with the cutter of biopsy device (10). In this instance, the channel (1698) of cassette insert (1690) that is aligned with access port (1819) is able to receive tissue samples from the cutter of biopsy device (10).

Although not shown, it should be understood that rotatable member (1820) may be rotated relative to probe seal (70) in an any order than that depicted and described above. It will also be apparent to those of ordinary skill in the art that an operator may cease rotating rotatable member (1820) relative to probe seal (70) once a sufficient number of tissue samples (90) have been deposited within tissue processing cassette (200). In other words, although tissue sample holder assembly (1800) is described above as being used to collect a tissue sample (90) in each channel (1698) of cassette insert (1690), it should be understood that in some uses it may be desirable to only collect samples (90) into one or more specific channels (1698) of cassette insert (1690). Accordingly, in some uses rotatable member (1820) may be rotated to skip some channels (1698). Similarly, it should be understood that an operator is not required to further rotate rotatable member (1820) through to each access port (1817, 1818, 1819) iteration. Rather, an operator may simply cease rotation rotating member (1820) when the procedure is completed. It should also be understood that tissue processing cassette (200) may be removed from manifold (1822) at any point during the engagement of tissue sample holder assembly (1800).

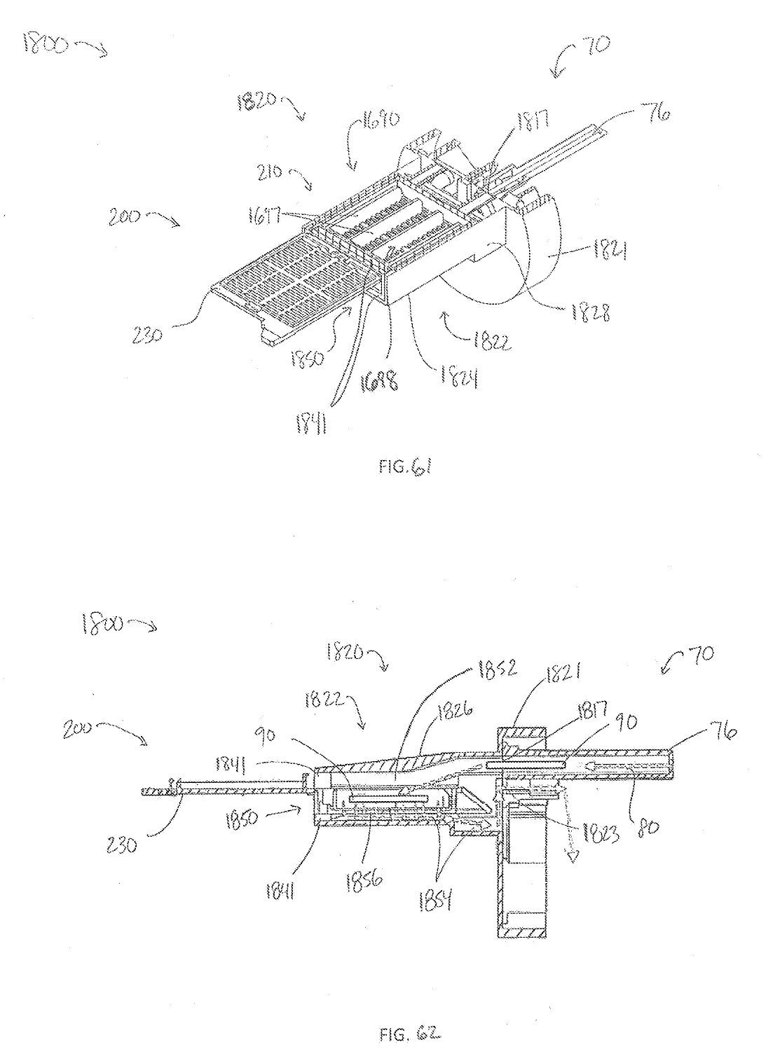

FIG. 62 shows a path (80) of a tissue sample (90) traveling through elongated shaft (76) towards cylindrically-shaped body (1821) until arriving at either access port (1817, 1818, 1819). In this instance, with a particular access port (1817, 1818, 1819) of manifold (1822) aligned with the cutter of biopsy device (10), tissue sample (90) enters manifold (1822) of rotatable member (1820) and is directed through the channel of access port (1817, 1818, 1819) until encountering tissue processing cassette (200). In other words, it should be understood that when tissue sample (90) is received within manifold (1822), tissue sample (90) does not proceed directly into channel (1698) of cassette insert (1690). Instead, tissue sample (90) first enters a channel defined commencing at access port (1817, 1818, 1819) and defined by upper wall (1826). The downward angle of upper wall (1826) towards tissue processing cassette (200) directs tissue sample (90) into the corresponding channel (1698) of cassette insert (1690) that is in alignment with that access port (1817, 1818, 1819) that is in current communication with elongate shaft (76). Thus, it should be understood that the angle of upper wall (1826) acts as a tissue sample deflector, director, or channeler to direct tissue sample (90) into tissue processing cassette (200) after tissue sample (90) is received through access port (1817, 1818, 1819). Accordingly, although upper wall (1826) is shown as having a specific angle and/or geometry, it should be understood that the particular configuration of upper wall (1826) may vary based on a number of considerations such as the positioning of access ports (1817, 1818, 1819) relative to manifold (1822), the velocity of tissue sample (90) transported from elongated shaft (76), the size of tissue samples (90), the gage size of needle (22), and/or etc.

With tissue sample (90) deposited within tissue processing cassette (200), channel (1698) of cassette insert (1690) isolates tissue sample (90) from any other tissue samples (90) previously deposited, or to be deposited, within tissue processing cassette (200). Seal (1841) forms an airtight seal against tissue processing cassette (200) while tissue processing cassette (200) is contained within manifold (1822). In this instance, seal (1841) abuts a proximal portion of tissue processing cassette (200) and prevents any fluid and/or tissue samples (90) from exiting tissue processing cassette (200).

Within inner chamber (1852), lower wall (1824) includes a vacuum opening (1856) and a vacuum chamber (1854). Vacuum opening (1856) extends along lower wall (1824) in parallel alignment with tissue processing cassette (200) when inserted into inner chamber (1852). Vacuum opening (1856) does not extend along the entire longitudinal length of tissue processing cassette (200) to thereby provide support for tissue processing cassette (200) when received within manifold (1822). Vacuum chamber (1854) similarly extends along lower wall (1824) and is in fluid communication with tissue processing cassette (200) through vents (224) and vacuum opening (1856). Vacuum chamber (1854) is in communication with vacuum opening (1856) to communicate vacuum from probe assembly (20) into tissue processing cassette (200). Vacuum enters manifold (1822) through vacuum opening (1856) that is in communication with vacuum port (1823). Next, vacuum travels through vacuum chamber (1854) and upwardly through vents (224) of tissue processing cassette (200). Vacuum then travels through corresponding vents (1695) of cassette insert (1690) and into inner chamber (1852) of manifold (1822). In this instance, the vacuum is now in communication with the particular access port (1817, 1818, 1819) that is currently aligned with elongate shaft (76), thereby effectively being positioned in communication with the cutter of needle (22). Vacuum is then used to pull tissue sample (90) through the cutter of needle (22) and into the corresponding channel (1698) of cassette insert (1690).

Once a sample is received within channel (1698) of cassette insert (1690), rotatable member (1820) is translated to a subsequent position relative to probe assembly (20), as shown in FIGS. 60A-60D. This translation indexes the next successive access port (1817, 1818, 1819) and channel (1698) to receive a tissue sample (90) therein by being in communication with elongate shaft (76) as similarly described above. In this position, another tissue sample (90) may be collected in tissue processing cassette (200) corresponding to the selected access port (1817, 1818, 1819) of manifold (1822). Once channels (1690) of cassette insert (1690) are filled with the number of tissue samples (90) desired by an operator, tissue processing cassette (200) is removed from manifold (1822) by pulling lid (230) proximally to thereby extract base (210) from within inner chamber (1852). With tissue processing cassette (200) completely removed from rotatable member (1820), an operator may next desire to perform certain analysis on the collected tissue samples (90) as similarly described above.

### VII. Exemplary Combinations

The following examples relate to various non-exhaustive ways in which the teachings herein may be combined or applied. It should be understood that the following examples are not intended to restrict the coverage of any claims that may be presented at any time in this application or in subsequent filings of this application. No disclaimer is intended. The following examples are being provided for nothing more than merely illustrative purposes. It is contemplated that the various teachings herein may be arranged and applied in numerous other ways. It is also contemplated that some variations may omit certain features referred to in the below examples. Therefore, none of the aspects or features referred to below should be deemed critical unless otherwise explicitly indicated as such at a later date by the inventors or by a successor in interest to the inventors. If any claims are presented in this application or in subsequent filings related to this application that include additional features beyond those referred to below, those additional features shall not be presumed to have been added for any reason relating to patentability.

### Example 1

A sample collection system for use with a biopsy device, wherein the system comprises: a tissue sample holder assembly including a slidable container and an indexing mechanism, wherein the slidable container defines a housing, wherein the indexing mechanism comprises a track and an actuator, wherein the actuator is selectively maneuverable, wherein the actuator is configured to slidably translate the slidable container within the track; a cassette tray defining a plurality of tissue sample chambers, wherein the cassette tray is configured for receipt within the housing of the tissue sample holder assembly such that the indexing mechanism is operable to translate the cassette tray within the track.

### Example 2

The sample collection system of Example 1, wherein the indexing mechanism includes a gear, wherein the gear is configured to mate with at least a portion of the slidable container.

### Example 3

The sample collection system of Example 2, wherein the gear is rotatably coupled to the actuator such that the gear is configured to rotate in response to actuation of the actuator.

### Example 4

The sample collection system of any one or more of Examples 2 through 3, wherein the actuator is operable to rotate relative to the tissue sample holder, wherein the actuator is configured to simultaneously rotate the gear in response to rotation of the actuator.

### Example 5

The sample collection system of Example 4, wherein the slidable container is configured to translate in the track in response to rotation of the gear such that the actuator is operable to move the slidable container.

### Example 6

The sample collection system of Example 5, wherein the gear includes a plurality of teeth extending laterally outward from the gear.

### Example 7

The sample collection system of any one or more of Examples 1 through 6, wherein the slidable container includes a plurality of teeth extending along at least a portion of the slidable member.

### Example 8

The sample collection system of any one or more of Examples 2 through 6, wherein at least a portion of the gear is positioned within the track.

### Example 9

The sample collection system of any one or more of Example 6 through 8, wherein at least a portion of the plurality of teeth of the gear are configured to extend into the track.

### Example 10

The sample collection system of any one or more of Examples 7 or 9, wherein the plurality of teeth of the gear are operable to mesh with the plurality of teeth of the slidable container such that the gear is in communication with the slidable container.

### Example 11

The sample collection system of Example 10, wherein the gear is configured to translate the slidable container in response to rotation of the plurality of teeth of the gear when meshed with the plurality of teeth of the slidable container.

### Example 12

The sample collection system of any one or more of Examples 1 through 11, wherein the indexing mechanism includes a plurality of slots.

### Example 13

The sample collection system of Example 12, wherein the plurality of slots are positioned about the tissue sample holder assembly at different locations relative to the gear.

### Example 14

The sample collection system of any one or more of Examples 12 through 13, wherein the actuator is configured to releasably engage the plurality of slots.

### Example 15

The sample collection system of any one or more of Examples 12 through 14, wherein the actuator comprises a flexible detent.

### Example 16

The sample collection system of any one or more of Examples 12 through 15, wherein the flexible detent is configured to engage the plurality of slots in response to rotation of the actuator and gear.

### Example 17

The sample collection system of Example 16, wherein the flexible detent is configured to inhibit translation of the slidable container within the track when engaged with at least one of the plurality of slots.

### Example 18

The sample collection system of any one or more of Examples 1 through 17, wherein the tissue sample holder assembly is configured to couple to a biopsy device.

### Example 19

The sample collection system of Example 18, wherein the tissue sample holder assembly is operable to receive tissue samples from the biopsy device within the slidable container.

### Example 20

The sample collection system of any one or more of Examples 1 through 19, wherein the slidable container includes a plurality of openings.

### Example 21

The sample collection system of Example 20, wherein the plurality of openings are sized and shaped to receive tissue samples therethrough.

### Example 22

The sample collection system of any one or more of Examples 1 through 21, further comprising a cover, wherein the cover is configured to mate with the slidable container.

### Example 23

The sample collection system of Example 22, wherein the cover comprises a pair of flanges and a fastening mechanism.

### Example 24

The sample collection system of Example 23, wherein the pair of flanges are sized and shaped to slidably engage the slidable container.

### Example 25

The sample collection system of any one or more of Examples 23 through 24, wherein the fastening mechanism is configured to inhibit the translation of the slidable container within the pair of flanges.

### Example 26

The sample collection system of Example 25, wherein the cover includes a plurality of slots operable to provide fluid communication through the cover.

### Example 27

The sample collection system of any one or more of Examples 1 through 26, further comprising a transport jar filled with a fixative, wherein the transport jar is configured to receive the slidable container after receipt of the cassette tray.

### Example 28

A remote tissue collection system, comprising: a biopsy device; a tissue sample holder; and a coupling assembly; wherein the coupling assembly includes a first coupler, a second coupler, and a pair of tubes; wherein the first coupler is configured to receive a portion of the biopsy device and the second coupler is configured to receive a portion of the tissue sample holder, wherein the first and second couplers are configured to engage the pair of tubes such that the coupling assembly is operable to provide fluid communication between the biopsy device and the tissue sample holder through the pair of tubes, wherein the tissue sample holder comprises a plurality of chambers operable to receive tissue samples, wherein the plurality of chambers are configured to rotate within the second coupler; wherein the pair of tubes are selectively maneuverable such that that biopsy device and the tissue sample holder are movable relative to each other, wherein one of the pair tubes is operable to transport tissue samples from the biopsy device to the plurality of chambers and the other tube of the pair of tubes is operable to transport fluids from the plurality of chambers to the biopsy device.

### Example 29

The remote tissue collection system of Example 28, wherein the first and second tubes are similarly sized such that the first and second tubes have the same length.

### Example 30

The remote tissue collection system of any one or more of Examples 28 through 29, wherein the first and second tubes are formed of a flexible and/or elastic material such that the first and second tubes are malleable.

### Example 31

The remote tissue collection system of any one or more of Examples 28 through 30, wherein the first and second tubes are operably coupled to a vacuum source such that the first and second tubes are pressurized.

### Example 32

The remote tissue collection system of any one or more of Examples 28 through 31, wherein the first and second tubes are removably attached to the biopsy device and the tissue sample holder such that the first and second tubes are configured to be detached from the biopsy device and the tissue sample holder.

### Example 33

A sample collection system for use with a biopsy device, wherein the system comprises: a tissue sample holder assembly including a slidable container and a track, wherein the slidable container defines a housing, wherein the slidable container is selectively maneuverable such that the slidable container is configured to slidably translate within the track in response to actuation of the slidable container; and a cassette tray defining a plurality of tissue sample chambers, wherein the cassette tray is configured for receipt within the housing of the slidable container such that the slidable container is operable to translate the cassette tray through the track in response to actuation of the slidable container.

### Example 34

A biopsy device comprising (a) a needle; (b) a cutter, wherein the cutter is configured to cut tissue samples; (c) a probe assembly, wherein the probe assembly is in fluid communication with the needle such that the probe assembly is configured to receive the tissue samples extracted by the cutter; (d) a tissue cassette, wherein the tissue cassette includes a top tray and a bottom tray pivotably coupled at a hinge, wherein the tissue cassette is configured to hold tissue samples within the top and bottom trays; and (e) a tissue cassette holder, wherein the tissue cassette holder is coupled to the probe assembly such that the tissue cassette holder is in fluid communication with the probe assembly, wherein the tissue cassette holder is selectively movable relative to the probe assembly to communicate with the probe assembly and thereby receive the tissue samples cut by the cutter, wherein the tissue cassette holder is configured to selectively receive the tissue cassette such that the tissue cassette holder is operable to store the tissue samples received from the probe assembly into the tissue cassette.

### Example 35

The biopsy device of Example 34, wherein the tissue cassette holder includes a manifold and a coupler.

### Example 36

The biopsy device of Example 35, wherein the manifold defines a chamber and a chamber opening, wherein the chamber is sized and shaped to receive the tissue cassette and enclose the tissue cassette within the manifold.

### Example 37

The biopsy device of Example 36, wherein the chamber is sized and shaped to partially enclose the tissue cassette within the manifold such that the top tray of the tissue cassette is positioned exterior to the manifold.

### Example 38

The biopsy device of any one or more of Examples 36 through 37, wherein the chamber is sized and shaped to completely enclose the tissue cassette within the manifold such that the top and bottom trays of the tissue cassette are positioned interior of the manifold.

### Example 39

The biopsy device of any one or more of Examples 36 through 38, wherein the chamber opening includes a seal configured to generate a compression fit against the tissue cassette when the tissue cassette is inserted into the chamber.

### Example 40

The biopsy device of any one or more of Examples 35 through 39, wherein the coupler is configured to engage the probe assembly such that the coupler is operable to attach the manifold to the probe assembly.

### Example 41

The biopsy device of any one or more of Examples 35 through 40, wherein the coupler includes a conduit configured to establish fluid communication between the probe assembly and the manifold.

### Example 42

The biopsy device of any one or more of Examples 35 through 40, wherein the coupler includes a track configured to slidably receive the manifold to releasably attach the manifold to the coupler.

### Example 43

The biopsy device of Example 42, wherein the track includes a fastening mechanism.

### Example 44

The biopsy device of Example 43, wherein the fastening mechanism includes a pair of flanges extending along the track.

### Example 45

The biopsy device of Example 44, wherein the pair of flanges are sized and shaped to receive the manifold therebetween such that the pair of flanges are configured to grasp the manifold to the coupler.

### Example 46

The biopsy device of any one or more of Examples 43 through 45, wherein the fastening mechanism includes at least one detent feature configured to releasably lock the manifold at a position relative to the track.

### Example 47

The biopsy device of Example 46, wherein the at least one detent feature is positioned along the track in alignment with an access port of the coupler such that the at least one detent feature is configured to selectively lock the manifold at the access port.

### Example 48

The biopsy device of any one or more of Examples 46 through 47, wherein the at least one detent feature is operable to disengage the manifold in response to a predetermined lateral force applied to the manifold.

### Example 49

The biopsy device of any one or more of Examples 35 through 48, wherein the manifold includes an angled top wall configured to divert the tissue samples received therein.

### Example 50

The biopsy device of any one or more of Examples 35 through 49, wherein the manifold includes at least one raised connector.

### Example 51

The biopsy device of Example 50, wherein the at least one raised connector is configured to releasably engage the at least one detent feature.

### Example 52

The biopsy device of any one or more of Examples 35 through 51, wherein the manifold includes at least one access channel configured to receive tissue samples from the probe assembly.

### Example 53

The biopsy device of Example 52, wherein the at least one access channel is operable to receive the tissue samples through the access port.

### Example 54

The biopsy device of any one or more of Examples 19.52 through 20.53, wherein the manifold includes three access channels.

### Example 55

The biopsy device of Example 54, wherein the manifold is configured to selectively translate between the three access channels to thereby provide fluid communication between a particular access channel of the three access channels and the probe assembly.

### Example 56

The biopsy device of any one or more of Examples 34 through 55, further including a first insert tray sized and shaped to fit within the tissue cassette.

### Example 57

The biopsy device of Example 56, wherein the first insert tray is configured to be removably received in the tissue cassette when the tissue cassette is positioned within the chamber of the manifold.

### Example 58

The biopsy device of any one or more of Examples 56 through 57, wherein the first insert tray includes at least one dividing wall.

### Example 59

The biopsy device of Example 58, wherein the at least one dividing wall is configured to form at least two channels within the tissue cassette.

### Example 60

The biopsy device of Example 59, wherein the at least two channels are sized and shaped to receive at least one tissue sample therein.

### Example 61

The biopsy device of any one or more of Examples 56 through 60, wherein the first insert tray includes a plurality of vent openings.

### Example 62

The biopsy device of any one or more of Examples 56 through 61, further including a second insert tray sized and shaped to fit within the chamber of the manifold.

### Example 63

The biopsy device of Example 62, wherein the second insert tray is configured to be removably received within the manifold with the manifold engaged to the coupler and the tissue cassette received within the manifold.

### Example 64

The biopsy device of any one or more of Examples 62 through 63, wherein the second insert tray includes at least one divider.

### Example 65

The biopsy device of Example 64, wherein the at least one divider is configured to form at least two chambers within the chamber of the manifold.

### Example 66

The biopsy device of Example 65, wherein the at least two chambers are sized and shaped to transfer at least one tissue sample therethrough.

### Example 67

The biopsy device of any one or more of Examples 56 through 66, wherein the at least two channels of the first insert tray and the at least two chambers of the second insert tray are similarly sized.

### Example 68

The biopsy device of any one or more of Examples 56 through 67, wherein the first insert tray includes a number of channels in accordance with the number of chambers in the second insert tray.

### Example 69

The biopsy device of any one or more of Examples 56 through 68, wherein the at least one dividing wall of the first insert tray and the at least one divider of the second insert tray are sized and shaped to align within the chamber when the first and second insert trays are positioned within the manifold.

### Example 70

The biopsy device of any one or more of Examples 35 through 69, wherein the manifold includes a receiving tray configured to removably translate within the manifold such that the receiving tray is sized and shaped to fit within the chamber through the chamber opening, wherein the receiving tray is configured to receive the tissue cassette such that the tissue cassette is removably translatable into the manifold.

Having shown and described various embodiments of the present invention, further adaptations of the methods and systems described herein may be accomplished by appropriate modifications by one of ordinary skill in the art without departing from the scope of the present invention. Several of such potential modifications have been mentioned, and others will be apparent to those skilled in the art. For instance, the examples, embodiments, geometrics, materials, dimensions, ratios, steps, and the like discussed above are illustrative and are not required. Accordingly, the scope of the present invention should be considered in terms of the following claims and is understood not to be limited to the details of structure and operation shown and described in the specification and drawings.

It should be understood that any of the versions of instruments described herein may include various other features in addition to or in lieu of those described above. By way of example only, any of the instruments described herein may also include one or more of the various features disclosed in any of the various references that are incorporated by reference herein. It should also be understood that the teachings herein may be readily applied to any of the instruments described in any of the other references cited herein, such that the teachings herein may be readily combined with the teachings of any of the references cited herein in numerous ways. Other types of instruments into which the teachings herein may be incorporated will be apparent to those of ordinary skill in the art.

It should be appreciated that any patent, publication, or other disclosure material, in whole or in part, that is said to be incorporated by reference herein is incorporated herein only to the extent that the incorporated material does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

We claim:

1. A sample collection and processing apparatus comprising:
a tissue processing cassette sized to be used in a pathology laboratory for purposes of dehydrating, embedding and sectioning, the tissue processing cassette having at least one distal opening through which tissue samples are received and having a floor with a plurality of openings; and
a biopsy device for cutting tissue samples and having a cassette holder configured to translate relative to a proximal end of the biopsy device and adapted to receive the tissue processing cassette, the cassette holder having a fluid path adapted to be coupled to a vacuum source and in fluid communication with the interior of the tissue processing cassette through the openings of the floor such that the vacuum from the vacuum source facilitates the transfer of the cut tissue samples into the received tissue processing cassette.

2. The apparatus of claim 1, wherein the tissue processing cassette includes a plurality of distal openings.

3. The apparatus of claim 1, wherein the tissue processing cassette includes a plurality of distal openings, wherein the cassette holder is translatable relative to a cutter of the biopsy device to index each distal opening with the cutter.

4. The apparatus of claim 1, wherein the plurality of openings of the tissue processing cassette are defined by a plurality of square-shaped openings in the floor of the tissue processing cassette.

5. The apparatus of claim 1, wherein the cassette holder defines a plurality of sample openings, wherein the tissue processing cassette defines a plurality of distal openings with each distal opening corresponding to a sample opening of the cassette holder, wherein the cassette holder is translatable relative to a cutter of the biopsy device to successively index each sample opening with the cutter.

6. The apparatus of claim 5, wherein each sample opening of the cassette holder is adjacent to another sample opening such that the plurality of sample openings are together arranged along a common axis.

7. The apparatus of claim 5, wherein the cassette holder further includes a plurality of vacuum openings, wherein each vacuum opening is proximate to and corresponds to a sample opening.

8. The apparatus of claim 7, wherein the cassette holder further includes a plurality of vacuum channels configured to direct vacuum from each vacuum opening and through the floor of the tissue processing cassette.

9. The apparatus of claim 1, wherein the cassette holder defines an angled portion, wherein the angled portion is configured to deflect one or more tissue samples into the tissue processing cassette when the tissue processing cassette is disposed in the cassette holder.

10. The apparatus of claim 1, wherein the tissue processing cassette includes an insert removably received within the interior of the tissue processing cassette, wherein the insert is configured to define a plurality of tissue sample chambers within the tissue processing cassette when the insert is received therein.

11. The apparatus of claim 1, wherein the tissue processing cassette includes an angled identification area configured to receive identifying information thereon.

12. The apparatus of claim 1, wherein the tissue processing cassette includes a lid connected to a base of the tissue processing cassette with a hinge.

13. The apparatus of claim 1, wherein the tissue processing cassette includes a plurality of interior walls, wherein the interior walls define a plurality of tissue sample chambers with each tissue sample chamber configured to receive a tissue sample.

14. The apparatus of claim 1, further comprising a coupler configured to couple the cassette holder to a proximal end of the biopsy device, wherein the coupler includes a track configured to slidably receive the cassette holder.

15. The apparatus of claim 14, wherein the coupler further includes an indexer configured to bias the cassette holder towards a plurality of discrete translational positions relative to the coupler.

16. A sample collection and processing apparatus for use with a biopsy device, wherein the system comprises:
a tissue sample holder assembly including a slidable member defining a mount, wherein the mount includes a cassette chamber and a vacuum chamber oriented below the cassette chamber; and
a tissue processing cassette sized to be used in a pathology laboratory for purposes of dehydrating, embedding and sectioning, the tissue processing cassette having a plurality of tissue sample chambers, wherein the mount is configured to receive the tissue processing cassette within the cassette chamber such that tissue samples are received from the biopsy device in different tissue sample chambers as the slidable member translates under vacuum supplied to the vacuum chamber.

17. The apparatus of claim 16, wherein the vacuum chamber of the mount includes a plurality of walls configured to direct vacuum into a particular tissue sample chamber of the tissue processing cassette.

18. The apparatus of claim 17, wherein the tissue processing cassette includes a plurality of internal walls defining each tissue sample chamber of the plurality of tissue sample chambers, wherein each internal wall of the tissue processing cassette corresponds to each wall of the mount.

19. The apparatus of claim 18, wherein the tissue sample holder defines a plurality of sample openings and a plurality of vacuum openings, wherein each vacuum opening is disposed between a wall of the mount, wherein each sample opening is in communication with each tissue sample chamber of the tissue processing cassette when the tissue processing cassette is received within the mount.

20. A method of collecting samples for analysis by a pathology laboratory, the method comprising:
attaching to a biopsy device a tissue processing cassette sized to be used in a pathology laboratory for purposes of dehydrating, embedding, and sectioning;
cutting a tissue sample with the tissue processing cassette attached to the biopsy device so as to receive the cut tissue sample into the attached tissue processing cassette under the direction of vacuum applied to a bottom surface of the tissue processing cassette;
translating the cassette relative to a cutter of the biopsy device to receive another cut tissue sample into the attached tissue processing cassette;
detaching the tissue processing cassette from the biopsy device; and
inserting the tissue processing cassette with the cut tissue sample into a container containing a fixing agent.
